(19) United States
(12) Patent Application Publication
(10) Pub. No.: US 2025/0059596 A1
(43) Pub. Date: Feb. 20, 2025

(54) METHODS AND COMPOSITIONS FOR METHYLATION AND CHEMICALLY MODIFYING ASSISTED TAILORABLE ENDS

(71) Applicant: THE REGENTS OF THE UNIVERSITY OF CALIFORNIA, Oakland, CA (US)

(72) Inventor: Robert EVANS, Berkeley, CA (US)

(21) Appl. No.: 18/787,942

(22) Filed: Jul. 29, 2024

Related U.S. Application Data
(60) Provisional application No. 63/516,411, filed on Jul. 28, 2023.

(51) Int. Cl. C12Q 1/6853; C12Q 1/6806

(52) U.S. Cl. CPC C12Q 1/6853; C12Q 1/6806

(57) ABSTRACT

The present invention provides for methods and compositions for one-pot Golden Gate cloning, or conventional two-pot restriction enzyme cloning, regardless of internal restriction sites. It uses modified nucleotides during PCR of DNA, or through post-hoc enzymatic modification of already existing DNA, wherein the modification blocks the internal restriction sites while leaving the flanking restriction sites unblocked.

### CROSS REFERENCE TO RELATED APPLICATIONS

This application claims priority to U.S. Provisional Patent Application Ser. No. 63/516,411, filed Jul. 28, 2023, which are hereby incorporated by reference.

### STATEMENT OF GOVERNMENTAL SUPPORT

The invention was made with government support under Contract No. DE-AC02-05CH11231 awarded by the U.S. Department of Energy. The government has certain rights in the invention.

### SUBMISSION OF SEQUENCE LISTING ON XML FILE

The instant application contains a Sequence Listing which has been submitted electronically in XML format and is hereby incorporated by reference in its entirety. Said XML copy, created on 2024-09-24, is named “2023-035-02 Sequence Listing.xml” and is 128 kilobytes in size.

### FIELD OF THE INVENTION

This invention relates generally to assemble of DNA parts.

### BACKGROUND OF THE INVENTION

REBASE (Roberts, et al., 2023) reports a variety of DNA base modifications and a non-exhaustive list of their effects on the cleavage of DNA by restriction enzymes, including complete blocking of digestion. PCR with the dGTP analog 7-deaza-dGTP has been known to block restriction digest by a variety of enzymes for over 30 years (Grime, et al., 1991). Likewise, PCR with 5-methyl-dCTP has also been shown to block complete restriction digest (Wong, et al., 1997).

As Type IIS restriction enzyme recognition sequences are non-palindromic, the distribution of the four nucleotides will be biased to one strand or the other, with a number of Type IIS enzymes completely lacking one or more nucleotides on one strand while having those nucleotides on the other strand. REBASE also shows that, for some modifications, there is a position-specific effect on restriction digestions.

PCR provides a means to incorporate chemically modified nucleotides in DNA while incorporating modified nucleotides only on the complementary strand of the primer sequence. These properties of Type IIS enzymes, and PCR with modified nucleotides and unmodified primers containing primer-borne restriction enzymes sites, may enable the restriction-enzyme cloning of DNA parts which contain internal restriction enzyme sites.

While historically PCR has been disfavored for generating larger DNA parts for construct assembly over the use of sequence-verified pre-cloned DNA parts, due to the possibility of introducing mutations, the development of very high-fidelity polymerases significantly decreases the likelihood of PCR-generated mutations (Pezza, et al.; Hadigol and Khiabanian, 2018). When high-fidelity PCR is done using error-free templates such as sequence verified clones or genomic DNA, even relatively large constructs on the order of tens of kilobases can be assembled using amplified parts.

The earlier cited work on PCR with modified nucleotides was performed with low-fidelity Taq polymerase, which is permissive for various non-canonical nucleotides. I want to maximize the likelihood of identifying both modified nucleotides that will block a number of Type IIS restriction enzymes, as well as high-fidelity polymerases that will amplify DNA with those modified nucleotides. To do this, in this chapter I've tested PCR amplification of 4 targets from 2 sequenced plasmids available in the lab using a variety of nucleotide analogs and high-fidelity polymerases.

Current methods require using restriction enzyme independent assembly techniques, or more temperamental or complex optimizations of restriction enzyme assembly, in order to assemble DNA parts with internal restriction sites.

### SUMMARY OF INVENTION

The present invention provides for methods and compositions for one-pot Golden Gate cloning, or conventional two-pot restriction enzyme cloning, regardless of internal restriction sites. It uses modified nucleotides during PCR of DNA, or through post-hoc enzymatic modification of already existing DNA, wherein the modification blocks the internal restriction sites while leaving the flanking restriction sites unblocked.

The present invention provides for techniques which silence internal restriction enzyme recognition sites while allowing assembly with the same restriction enzyme at non-silenced sites. One technique involves PCR amplification of assembly parts with unmodified primers and modified dNTPs. This results in nucleoside modified assembly parts which silence internal restriction enzyme recognition while leaving the unmodified terminal recognition sites cleavable. The other technique involves methyltransferase-driven modification of assembly parts without PCR to silence the internal recognition sites, ligation of unmodified adapters containing non-silenced sites, and subsequent assembly at these non-silenced sites.

In some embodiments, the invention is a method for one-pot Golden Gate cloning, or conventional two-pot restriction enzyme cloning, regardless of internal restriction sites, comprising: using modified nucleotides during PCR of a DNA, or through post-hoc enzymatic modification of already existing DNA, such that the use of the modified nucleotides blocks the internal restriction sites while leaving the flanking restriction sites unblocked.

In some embodiments, the method comprises methylating or modifying one or more nucleotides, or other nucleotide modification, to modify one or more internal restriction sites while leaving flanking sites digestible. In some embodiments, the method comprises silencing of internal sites while retaining digestible flanking sites in the primers, and/or ligating unmodified adapters. In some embodiments, the methylating or modifying comprises introducing or adding a methyltransferase. Suitable methyltransferases for the methylation include CpG methyltransferase, which catalyzes CG into ^{5m}CG; GpC methyltransferase, which catalyzes GC into G^{5m}C; and EcoGII methyltransferase, which catalyzes A into^{N6m}A In some embodiments, the method comprises carrying out PCR using one or more modified nucleotides, such as dUTP, 7-deaza-dGTP, and 5-methyl-dCTP.

The present invention provides for a kit comprising one or more compositions, components, enzymes, primers, oligos, or the like as described herein, sufficient for carrying out the method of the present invention.

In some embodiments, the kit comprising one or more of the following: T4 DNA Ligase Buffer, pGGAselect (Golden Gate destination plasmid, CamR), one or more plasmids carrying fragments of a gene cassette encoding a reporter gene, Golden Gate Enzyme Mix (such as BsmBI-v2), T4 DNA Ligase, and BsmBI-v2, BsaI-HIF-v2 or any other suitable restriction enzyme, such as a Type IIS restriction enzyme.

Suitable Type II enzymes (Type IIS) include FokI, AlwI, and BsaI, which cleave outside of their recognition sequence to one side. These enzymes are intermediate in size, 400-650 amino acids in length, and they recognize sequences that are continuous and asymmetric. They comprise two distinct domains, one for DNA binding, the other for DNA cleavage. They are thought to bind to DNA as monomers for the most part, but to cleave DNA cooperatively, through dimerization of the cleavage domains of adjacent enzyme molecules. For this reason, some Type IIS enzymes are much more active on DNA molecules that contain multiple recognition sites. Type IIS restriction enzyme can generate copious different overhangs on the inserts and the vector; for instance, BsaI creates 256 four-basepair overhangs.

The present invention comprises one or more method steps and/or components described in NEB® Golden Gate Assembly Kit (BsmBI-v2) NEB #E1602S/L Instruction Manual, herein incorporated by reference.

The present invention provides for compositions, systems, devices, methods and steps described herein.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. The results demonstrate that, as expected, only Q5U and Taq could amplify DNA with dUTP, though with noticeably lower band intensities. All of the polymerases which amplified using dNTPs could also amplify with 7dGTP, though fluorescent quenching resulted in faint bands. And only Kapa HiFi and Taq could amplify DNA with 4mCTP. Further testing with Smo HiFi, Taq, and Phire was abandoned. Ladder is the Thermo GeneRuler DNA mix. Polymerase conditions tested: SH=Smo-HiFi; SHD=Smo-HiFi+10% DMSO; SG=Smo G-HiFi; Q5=Q5 Hot Start; Q5U=Q5U Hot Start; P=Phusion Hot Start II; SF2=SuperFi II; K=Kapa HiFi; T=GoTaq G2; Ph=Phire Hot Start II. For all images lane 1 is PCR with regular dNTPs and lane 2 is with an alternative nucleotide: A) dUTP, B) 4mCTP, C) 5mCTP, and D) 7dGTP. Following the initial test, cycling conditions were restored to the normal 3-step protocol to both see if the lower 2-step temperature is a requirement for amplification with 4mCTP or 7dGTP, and to ensure specificity of amplification.

FIG. 2. Amplification was shown to mostly fail for 4mCTP at higher denaturing, annealing, and extension temperatures, though very faint amplification was seen with SuperFi II when the annealing temperature was 65° C. for 30 seconds.

FIG. 3. Comparison of Q5U to Phusion U for dUTP PCR demonstrates amplification failure with Phusion U.

FIG. 4. Successful PCR of a 2731 bp target with dUTP, 7dGTP, 5mCTP, and PCR failure with 7dATP, and 6mATP.

FIG. 5. Methylation and restriction digestion of three linearized plasmids shows the effects of CpG, GpC, and EcoGII methylation on digestion by various Type IIS restriction enzymes.

FIG. 6. DNA amplified with dUTP blocks BsmBI, Esp3I, EarI, and BfuAI and inhibits SapI; 4mCTP inhibits LguI and blocks the other enzymes.

FIG. 7. Restriction digestion of dNTP, dUTP, 5mCTP, 7dGTP, CpG methylated, GpC methylated, and EcoGII methylated 2731 bp amplicon.

FIG. 8. Transformation of pENTR-SD-SmaI and 346-6-GG-mod recircularization reactions into DH10β(Δung) shows some rescue of uracil modified vectors. A) From left to right 100 ng of the linear pENTR-SD-SmaI amplified with dUTP, dNTPs, 7dGTP, 5mCTP, GpC methylated, CpG methylated, ladder, 346-6-GG-mod undigested GpC, CpG, EcoGII and ZraI linearized GpC, CpG, and EcoGII. B) and C) Transformation plates of recircularized vectors. B) Transformations outgrown for 1 hour at 37° C. and 1/150^{th} was plated. Left image, clockwise from top left: pENTR-SD-SmaI CpG, GpC, dNTPs; right image, clockwise from top left: 346-6-GG-mod undigested GpC, CpG, linearized CpG, GpC. Approximately equal number of colonies per plasmid. C) Transformations outgrown for 3 hours at 30° C. and ⅔^{rds} was plated. Left image, clockwise from top left: pENTR-SD-SmaI dNTPs (lawn), dUTP (360 Colony Forming Units [CFUs]), 5mCTP (136 CFUs), and 7dGTP (2 CFUs); right image, clockwise from top left: 346-6-GG-mod undigested CpG (lawn), linearized CpG (lawn), linearized EcoGII (58 CFUs), undigested EcoGII (46 CFUs).

FIG. 9. R6k-to-346 PCR screening of Top10 colonies. The ladder is Thermo's GeneRuler DNA mix. Expected bands are 3593 bp for single insert containing clones of the PaqCI, BbsI, BsaI, Esp3I, and BsmBI reactions. 3625 bp for single insert containing clones of LguI and EarI reactions, and 1981 bp for empty vector. The LguI and EarI reactions can ligate multiple full-length inserts. The inserts with internal restriction sites might have unusual sized bands if they mis-ligate on the internal restriction site overhangs.

FIG. 10. R6k-to-346 DH10β(Δung) Golden Gate PCR screens. Ladder is Thermo's GeneRuler DNA mix. A) The PaqCI assemblies were test plated and 4 colonies each picked. dNTP and dUTP colonies all show a correct band around 3593 bp. For the 7dGTP and 5mCTP a correct band would be around 2582 bp with an incorrect band of 970 bp. B) and C) A maximum of two colonies of each transformation was screened. B) Left: PaqCI and BbsI colony screens interlaced (dNTP, dUTP, 7dGTP, 5mCTP, vector only). Right: BsaI and BsmBI colony screens interlaced. Sent for sequencing: PaqCI dUTP and 5mCTP, BsaI dUTP, BsmBI 5mCTP colonies. C) Left: Esp3I and EarI colony screens interlaced. Right: LguI colony screens. Sent for sequencing: One each of the Esp3I dNTP and dUTP, EarI dUTP and 5mCTP, one LguI dNTP, and the LguI dUTP and 5mCTP colonies.

FIG. 11. Direct PCR of R6k-to-346 one-pot Golden Gate reactions showing assembly failures and successes. Ladder is Thermo's GeneRuler DNA mix. Red arrow indicates a successful assembly, the top white arrow indicates recircularized vector, and the bottom two indicate BbsI misassemblies. A) Assemblies are intercalated, with the left enzyme in the left lane and the right enzyme in the right lane. Insert modification is indicated below: N=unmodified, U=dUTP, 7=7dGTP, 5=5mCTP. Left to right the controls are vector only+PaqCI, +BbsI-HF, +BsaI-HFv2, +BsmBI-v2, +Esp3I, +EarI, +LguI, and undigested vector. B) Q5U rescreens of the dUTP assemblies. Left to right: PaqCI, PaqCI vector only control, BbsI-HF, BbsI-HF vector only control, BsaI-HFv2, BsaI-HFv2 vector only control, BsmBI-v2, BsmBI-v2 vector only control, Esp3I, Esp3I vector only control, EarI, EarI vector only control, and finally LguI.

FIG. 12. pKD46_SpCas9_EcgRNA plasmid map.

FIG. 13. E. coli DH10β ung gene context.

FIG. 14. Gel images of the cloning parts. The ladder is Thermo's GeneRuler DNA Ladder Mix. A) The left and right amplified homology arms to knock out the ung coding sequence, both approximately 680 bp in size. B) Rightmost lane is a microliter of the PCR-fused homology arms, with the correct band on top at about 1.3 kb. C) Approximately 100 ng of the 13 kb AvrII-linearized pKD46-SpCas9-EcgRNA plasmid.

FIG. 15. Gel images of colonies containing pKD46-SpCas9-EcgRNA with the inserted homology arms. A) PCR screening of 32 colonies. The expected band is 1817 bp, which should fall between the 1.5 kb and 2.0 kb bands (tops of these ladder bands indicated by the red arrows). B) Aliquots of BsaI digests of the bulked-up midipreps of clones 11, 19, and 29.

FIG. 16. First colony screen of DH103 transformed with the spacer-ligated plasmids. The screening for the clones is set off by white lines. The first lane was screened with the ung.LarmF and ung.RarmR primers which should yield a product of 1236 bp from the plasmid (grey arrow), and 1925 bp from the genome if the ung CDS is still present (yellow arrow). The second lane was screened with the pKD.screenF/R primers which should yield a band of 1817 bp if the ung homology arms are present. Counting left to right, top to bottom, clones #1-3, 5-7, 9-10 show these expected patterns, with 9 and 10 no longer showing the genomic band. Additionally clones 8 and 19 show expected sized bands for an ung knockout or plasmid-borne homology arms, but do not show the expected sized band for the plasmid.

FIG. 17. Colony screening of single colonies from the plasmid curing plates. A) PCR screening of 32 colonies using the same primer pairs as FIG. 5. All but two clones show absence of the plasmid. A band of 1925 bp, indicated by the yellow arrow, shows an unedited ung CDS. A band of 1236 bp, indicated by the grey arrow, shows a deletion of the ung CDS. B) Clones 9-12, and 17-20 were screened with the ung-screen.F/R primers to yield a 1291 bp band. These PCRs were sent for sequencing to verify complete deletion of the ung CDS without other mutations.

FIG. 18. Representative sequence analysis of a DH10β(Δung) clone. A) Read alignments against the ung reference sequence showing the exact 690 bp deletion of ung with no extra mutations. B) Due to alignment issues over the gap there appears to be more reads on the left flank that the right, but inspection shows this is not the case (SEQ ID NO:1). C) ung coding sequence in uppercase with left and right flanks in lower case (SEQ ID NO.2).

FIG. 19. pCC1FosY-2LApR-PaqCI.

FIG. 20. pINT-Chlor-FP-LguI.

FIG. 21. 27868 bp BGC from Dickeya solani DSM 28711.

FIG. 22. Amplification with non-tailed primers shows specific amplification. Ladder is Thermo's GeneRuler DNA mix. A) Non-tailed PCR of BGC parts. The Ds8.A.2 reaction evaporated in the cycler. Ds8.A 5-7 and Ds8.B.7 may show a non-specific or primer dimer band just under the 100 bp mark. B) Repeat of Ds8.A.2 PCR reaction.

FIG. 23. Amplification of tailed parts required some optimization for some parts. One microliter of the 50 μL bead purified assembly reactions. The ladder is Thermo's GeneRuler DNA mix. P=PaqCI, Ba=BsaI, Bb=BbsI, Bm=BsmBI, L=LguI, 7=7dGTP, 5=5mCTP, U=dUTP, red text are amplification failures on the first round.

FIG. 24. Repeat amplification of failed reactions. Ladder is Thermo's GeneRuler DNA mix. A) The wrong oligos were used for A1_BsaI. B) Purification gel for A1, A1_BbsI, A1_BsmBI, and B1. C) Aliquots from the final PCR attempt. The left reaction for each is 1M betaine, the right is 1×GC enhancer. B1_dUTP, B1_LguI_dUTP, A1_dUTP, and A1_BsmBI_dUTP were abandoned for insufficient yield. B2_dUTP, B2_LguI_dUTP, A4_dUTP, and A1_BsaI reactions were run out for gel purification.

FIG. 25. Digestion of methyltransferase-protected pooled DNA shows a high degree of protection. Ladder is Thermo's GeneRuler DNA mix. Lane 1 is a PaqCI digest of the GpC-protected DNA. Non-protected DNA should give bands of 3302, 2544, 2241, 1876, 1147, and 884 bp. Lane 2 is an LguI digest of the GpC-protected DNA. Non-protected DNA should give bands of 3431, 3392, 2967, 989, 669, and 593 bp. Some degree of non-protection is visible. Lane 3 is a digest of the lane 2 DNA with EarI, which only protects the LguI sites from EarI digestion. Lane 4 is EcoGII-protected DNA digested with EarI, showing effectively complete protection. Lanes 5 and 6 show the CpG-protected DNA digested with BsmBI and Esp3I, respectively. Non-protected DNA would give bands of 4165, 3716, 1748, 1107, 712, 424, 364, and 21 bp.

FIG. 26. Efficiency of assembly reactions on a Sybr Gold gel. Ladder (L) is Lambda-HindIII digest: 23130, 9416, 6557, 4361, 2322, 2027, 564, 125 (not visible) bp. Yellow arrows indicate the unligated pCC1FosY-2LApR vector for the PaqCI, BsaI, and BsmBI reactions (top arrow), or the unligated pINT vector for the BbsI, EarI, and LguI reactions. ad=adapter-ligated amplicons, PCR=tailed PCR amplicons, N=unmodified, G=GpC methylated, A=EcoGII methylated, 5=5mCTP amplified, 7=7dGTP amplified,U=dUTP amplified, -ve=vector only control reactions.

FIG. 28. Initial screening of picked colonies to select ones for full screening. Ladder is Thermo's GeneRuler DNA mix. Screening shows intense Ds8.B.04 bands for 2/4 PaqCI adapter (GpC) colonies, 3/4 EarI PCR (5mCTP) colonies, 1/4 EarI PCR (dUTP) colony, and 1/4 LguI PCR (dNTP) colony. Faint bands are seen for 1/4 BsaI adapter (EcoGII) colony, 3/4 BsaI adapter (dNTP) colonies, 1/4 BsaI PCR (7dGTP) colony, 1/4 BsaI adapter (5mCTP) colony, 2/4 EarI PCR (dNTP) colonies, and 1/4 LguI PCR (dNTP) colonies. No other picked colonies showed presence of the part 4 amplicon.

FIG. 27. PCR screening of assemblies show which reactions resulted in probable full-length assemblies.

FIG. 29. Sequencing PCRs. Numbered colonies (#) and assembly reactions (assemb) were PCR screened with barcoded primer sets for sequencing. Ladder is Thermo's GeneRuler DNA mix. Red highlighted reactions did not show the expected band. Yellow highlighted reactions showed the expected band faintly. The image in the bottom right corner has the Phusion U screens of the dUTP assemblies. List of abbreviations: Paq=PaqCI, Bsa=BsaI-HFv2, Bsm=BsmBI-v2, Bbs=BbsI-HF, Ear=EarI, Lgu=LguI; ad=adapter-ligated, PCR=primer-tailed; G=GpC methylated, A=EcoGII methylated, 5=5mCTP modified,N=unmodified, 7=7dGTP modified, U=dUTP modified; ##=colony number, (assemb)=direct assembly reaction PCR screen. For the Phusion U image: 5′=5′ primer set, M=middle primer set, 3′=3′ primer set.

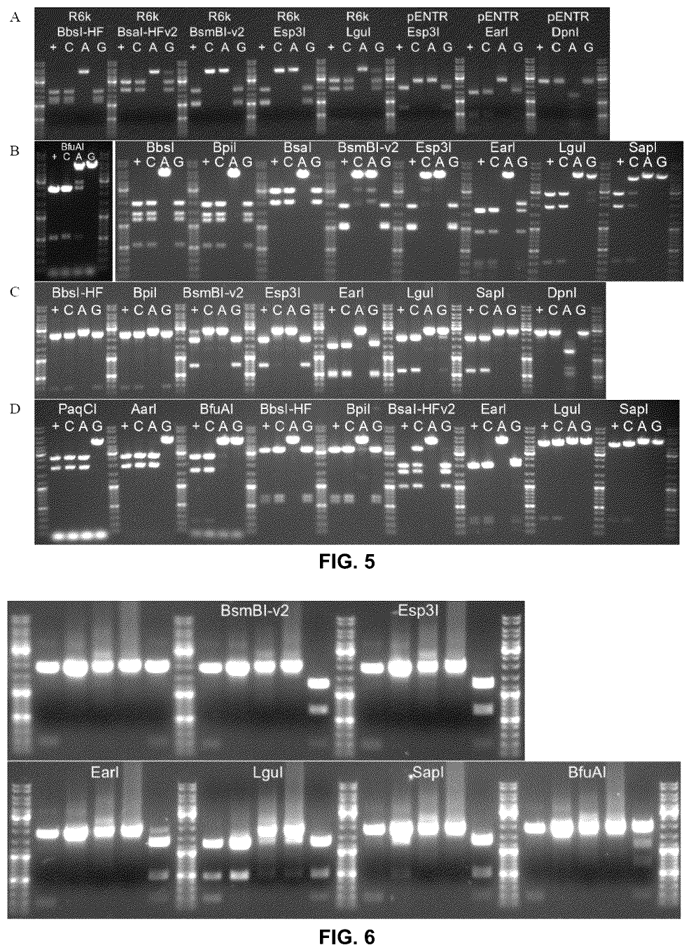

FIG. 30. Domestication of internal restriction sites (Engler, et al., 2008, FIG. 5A).

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

As used in the specification and the appended claims, the singular forms “a,” “an,” and “the” include plural referents unless the context clearly dictates otherwise. Thus, for example, reference to “molecules” includes a plurality of a molecule species as well as a plurality of molecules of different species.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The term “about” refers to a value including 10% more than the stated value and 10% less than the stated value.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

Based on prior art (MASTER cloning) this invention provides for two techniques and a hybrid of both techniques:

(1) Golden Gate or conventional restriction digest and ligation cloning of direct PCR products and additional DNA (such as a vector backbone). Some or all of the assembly parts being PCRed with modified nucleotides to silence internal sites while leaving the flanking sites present in the primers unmodified.

(2) Methylation of DNA parts with methyltransferases chosen to silence internal restriction sites. Followed by ligation of unmodified adapters which contain restriction sites for subsequent restriction digest and ligation (as one-pot Golden Gate reactions or as standard two-pot restriction digests followed by ligation).

(3) Both techniques together, as desired for particular DNA pieces.

This improves on the prior MASTER cloning protocol by allowing assembly using a variety of restriction enzymes, not limiting assembly to a single restriction enzyme. It requires fewer PCR steps compared to technique #1 of the MASTER protocol. It allows use of easier to use restriction enzymes then the single MspJI enzyme used in the MASTER strategy. It allows one-pot assembly, where the MASTER strategy requires two pots even for Golden Gate cloning.

Conception of the idea was based on an intuitive leap. This idea came about while thinking about MASTER cloning in which unmodified DNA is flanked by modified primers or adapters to allow site-specific digestion by a methylation requiring restriction enzymes, along with the knowledge that MASTER is problematic to get working (at least in my hands), and juxtaposed with the knowledge that nucleoside modification can block internal restriction sites.

There is some prior knowledge on how particular DNA methylation blocks particular restriction enzymes, but this information is incomplete. Additionally, while it's known that PCR with unnatural nucleotides (such as 7-deaza-dGTP) can block restriction sites, there's not a lot of information as to which enzymes are blocked by which modifications, especially when it comes to the type IIS enzymes used for Golden Gate cloning. It wasn't obvious until the studies were done which would be the best enzyme and modification combinations to make this work. It also isn't obvious whether technique 2 would be efficient, and the optimizations needed to make it efficient, especially for the cloning of multiple pieces. Additionally cloning tests showed that particular modification strategies greatly hinder DNA transformation or replication in the E. coli host under certain circumstances.

After the initial development of this technique I discovered prior art specifically using methylation to block restriction sites for Golden Gate assembly:
(1) MetClo: methylase-assisted hierarchical DNA assembly using a single type IIS restriction enzyme:
(2) Recursive DNA Assembly Using Protected Oligonucleotide Duplex Assisted Cloning (PODAC):
   Both techniques are for hierarchical assembly. While both use methylation as a modification tool, neither use PCR with modified oligos as a modification tool. Both also are at least partly dependent on the choice of restriction enzyme, while the technique presented here is mostly agnostic as to restriction enzyme choice. With the modification strategies used neither technique is applicable to the silencing of internal restrictions sites of parts.

Experiments so far have used non-optimal vectors and DNA parts which were on hand. Validation needs to be repeated with more optimal vectors and parts, and finally finished with testing of multi-part DNA assembly. Additional development is desired to show whether certain modification techniques can be used efficiently, or are non-starters (specifically cloning with dUTP and 7-deaza-dGTP amplified parts). Additional testing is needed to show which modifications work best for which restriction enzymes. It is assumed pre-hoc that PCR of parts with dUTP is best used on GC-rich DNA, and PCR with 5-methyl-dCTP or 7-deaza-dGTP is best used on AT-rich DNA, while all three of the modified nucleotides can be adequately used for PCR of AT/GC-balanced DNA, but this needs to be tested.

However, at this stage, for vector plus one-part DNA assembly, proof of concept has been shown sufficiently to go forward with actual production.

Synthetic biologists and synthetic DNA providers can use these techniques for fast and efficient assembly of DNA parts containing internal restriction sites, and are the most likely people to use it. This is the best use of the invention.

Reagent companies such as New England Biolabs and Thermo-Fisher can develop improved reagents (such as polymerases capable of both reading and incorporating modified nucleotides, or E. coli strains capable of amplifying uracil or 7-deaza-guanosine containing DNA) to extend upon this technique.

Golden Gate assembly is the DNA assembly technique least problematic with respect to repetitive DNA elements and secondary structure. It is also a preferred technique for hierarchical assembly. What it fails on is the presence of internal restriction enzyme recognition sites. The techniques presented here allow the user to ignore the presence of internal restriction sites. All the benefits of speedy Golden Gate assembly without the drawback.

Golden Gate cloning is a method of DNA assembly which uses Type IIS restriction enzymes to assemble DNA parts in a directional, scarless manner mostly independent of sequence context. It is especially useful for library and combinatorial DNA assembly, or the assembly of many parts. Golden Gate cloning is problematic, or impossible, when the chosen Type IIS enzyme has recognition sites internal to any of the parts being assembled, as internal sites can prevent parts from staying together through redigestion or may misligate on their overhangs to the wrong parts. FIG. 30 shows an example of the traditional Golden Gate strategy when internal sites are present, which is to domesticate, or silence, those sites through base changes. On the top image two internal BsaI restriction sites are represented with “B”. Primers are designed to split the DNA at these internal restriction sites with base changes in the primer tails to eliminate the sites. These primers include additional flanking BsaI sites and four base overhangs that allow restriction digestion and directional ligation of the parts into a vector. The cloned DNA is identical to the original DNA except for the base change mutations eliminating the internal restriction sites. The major drawbacks to this approach may be a prohibitive number of internal sites to domesticate, possible side-effects of domestication on the biological function of the DNA, and poor locations for specific primer design.

The research described in this thesis investigates the use of modified nucleotide analogs to block digestion of internal enzyme recognition sites while leaving the flanking sites active. I test two assembly methods, a number of nucleotide analogs and modifications, and a number of Type IIS restriction enzymes. This is done for four reasons: 1) I do not know what will work in advance; 2) I do not know the effects of these modifications on assembly efficiency and sequence fidelity in advance; 3) I want to parallel successful prior research so as to extend the usefulness of that research; 4) I want this research to be as useful as possible to others who may have their own constraints.

In one assembly method, the DNA parts are PCR amplified with modified nucleotides and primer-borne Type IIS recognition sites. The modified nucleotides block the internal recognition sites, but cannot block the sites on the unmodified primers. In the second method, PCR or treatment with a methyltransferase is used to block all enzyme recognition sites, and then adapters with unmodified recognition sites are ligated onto the DNA parts to allow digestion and assembly of the parts.

In Example 1, four alternative nucleotides, out of eight tested, are identified which can be successfully used to amplify DNA parts with several high-fidelity polymerases. Based on robustness and reproducibility of amplification, three of the four nucleotides are carried forward for further testing. These are dUTP, 5-methyl-dCTP, and 7-deaza-dGTP.

In Example, 2 DNA amplified with these alternative nucleotides, as well as DNA methylated with three methyltransferases, is tested for their ability to prevent digestion with a number of Type IIS restrictions enzymes. I then test the ability to clone this modified DNA, when the modifications are either in the vector backbone, or in an insert. Hypermethylation of cytosines or adenosines greatly inhibit colony formation when in the vector backbone, and this inhibition is lessened, though still present, when hypermethylation is in an insert. Complete substitution of thymine with uracil in the vector backbone likewise inhibits colony formation in a Δung cloning strain, though not as much as for hypermethylation, and has no effect when in an insert. Complete substitution of guanosine with 7-deaza-2-guanosine in the vector backbone effectively eliminates colonies. This may also be the case with 7-deaza-2-guanosine substituted inserts. CpG and GpC methylation have no apparent effect on colony formation. Various combinations of Type IIS enzymes and DNA modifications showed effective assembly strategies for most of the tested enzymes.

In Example 3, a DH10β(Δung) cloning strain, required for cloning of uracil-substituted DNA, is made to verify the gene knockout, and test transformation efficiency.

In Example 4, a variety of enzymes and modification cloning strategies are tested on a 7 part assembly of a 28 kb biosynthetic gene cluster. While testing was not robust, the most successful strategies were PaqCI using GpC methylated DNA ligated to adapters, and EarI with tailed primers and either 5-methyl-dCTP or dUTP amplified parts. Cloning into DH10β(Δung) is not too mutagenic for use in cloning, though evidence shows that too permissive PCR with 5-methyl-dCTP can cause a lot of C>T mutations which are not caused by the Uracil-DNA glycosylase knockout. The use of nucleotide modifications can enable robust Golden Gate assemblies and cloning of DNA constructs, regardless of internal restriction sites.

### REFERENCES CITED

Beck, R., & Burtscher, H. (1994) Introduction of arbitrary sequences into genes by use of class IIS restriction enzymes. Nucleic Acids Research, 22(5):886-7. doi: 10.1093/nar/22.5.886.
Blin, K., Shaw, S., Kloosterman, A. M., Charlop-Powers, Z., van Weezel, G. P., Medema, M. H., & Weber, T. (2021) antiSMASH 6.0: improving cluster detection and comparison capabilities. Nucleic Acids Research, doi: 10.1093/nar/gkab335.
Chen, W. H., Qin, Z. J., Wang, J., & Zhao, G. P. (2013) The MASTER (methylation-assisted tailorable ends rational) ligation method for seamless DNA assembly. Nucleic Acids Research, 41(8):e93. doi: 10.1093/nar/gkt122.
Dragan, A. I., Casas-Finet, J. R., Bishop, E. S., Strouse, R. J., Schenerman, M. A., & Geddes, C. D. (2010). Characterization of PicoGreen interaction with dsDNA and the origin of its fluorescence enhancement upon binding. Biophysical Journal, 99(9):3010-9. doi: 10.1016/j.bpj.2010.09.012.
Dudová, Z., Špaček, J., Havran, L., Pivoňková, H., & Fojta, M. (2016). Interactions of fluorescent dye SYBR Green I with natural and 7-deazaguanine-modified DNA studied by fluorescence and electrochemical methods. Monatshefte für Chemie/Chemical Monthly, 147, 13-20. doi: 10.1007/s00706-015-1578-5.
Duncan, B. K., & Weis, B. (1982). Specific mutator effects of ung (uracil-DNA glycosylase) mutations in Escherichia coli. Journal of Bacteriology, 151(2):750-55. doi: 10.1128/jb.151.2.750-755.1982
Ehrlich, M., Norris, K. F., Wang, R. Y., Kuo, K. C., & Gehrke, C. W. (1986) DNA cytosine methylation and heat-induced deamination. Bioscience Reports, 6(4):387-93. doi: 10.1007/BF01116426.
Engler, C., Kandzia, R., & Marillonnet, S. (2008) A one pot, one step, precision cloning method with high throughput capability. PLoS One, 3(11):e3647. doi: 10.1371/journal.pone.0003647.
Flores-Juárez, C. R., González-Jasso, E., Antaramian, A., & Pless, R. C. (2016). PCR amplification of GC-rich DNA regions using the nucleotide analog N4-methyl-2′-deoxycytidine 5′-triphosphate. Biotechniques, 61(4):175-182. doi: 10.2144/000114457.
Grime, S. K., Martin, R. L., & Holaway, B. L. (1991). Inhibition of restriction enzyme cleavage of DNA modified with 7-deaza-dGTP. Nucleic Acids Research, 19(10):2791. doi: 10.1093/nar/19.10.2791.
Hadigol, M., & Khiabanian, H. (2018). MERIT reveals the impact of genomic context on sequencing error rate in ultra-deep applications. BMC Bioinformatics, 19, 219. doi: 10.1186/s12859-018-2223-1.
Hayakawa, H., Kumura, K., & Sekiguchi, M. (1978) Role of uracil-DNA glycosylase in the repair of deaminated cytosine residues of DNA in Escherichia coli. The Journal of Biochemistry, 84(5):1155-64. doi: 10.1093/oxfordjournals.jbchem.a132231.
IDT-DNA. Oligoanalyzer Tool—Primer Analysis and TM Calculator: IDT Integrated DNA Technologies.
Kuipers, O. P. (1996). Random mutagenesis by using mixtures of dNTP and dITP in PCR. Methods in Molecular Biology 57: In Vitro Mutagenesis Protocols, 351-6. doi: 10.1385/0-89603-332-5:351.
Li, L. Y., Li, Q., Yu, Y. H., Zhong, M., Yang, L., Wu, Q. H., Qiu, Y. R., & Luo, S. Q. (2011) A primer design strategy for PCR amplification of GC-rich DNA sequences. Clinical Biochemistry, 44(8-9):692-8. doi: 10.1016/j.clinbiochem.2011.02.001.
Lin, D., & O'Callaghan, C. A. (2018) MetClo: methylase-assisted hierarchical DNA assembly using a single Type IIS restriction enzyme. Nucleic Acids Research, 46(19):e113. doi: 10.1093/nar/gky596.
Lindahl, T., & Nyberg, B. (1974) Heat-induced deamination of cytosine residues in deoxyribonucleic acid. Biochemistry, 13(16):3405-10. doi: 10.1021/bi00713a035.
Matsumura, I. (2022) Golden Gate assembly of BioBrick-compliant parts using type II restriction endonucleases. Biotechniques. 72(5):185-193. doi: 10.2144/btn-2021-0083.
NEB. Cleavage Close to the End of DNA Fragments. New England Biolabs. Webpage for: neb.com/tools-and-resources/usage-guidelines/cleavage-close-to-the-end-of-dna-fragments NEB. (2018) Joining of Difficult to Ligase dsDNA Fragments with Blunt/TA Ligase Master Mix. New England Biolabs. Application Note. Webpage for: neb.com/-/media/nebus/files/application-notes/appnote_joining_of_difficult_to_ligate_dsdna_fragments_with_blunt_ta_ligase_master _mix.pdf NEB. NEBridge Ligase Fidelity. New England Biolabs. Webpage for: ligasefidelity.neb.com NEB. Tm Calculator. New England Biolabs. Webpage for: tmcalculator.neb.com Nucleotide [Internet]. Bethesda (MD): National Library of Medicine (US), National Center for Biotechnology Information; [1988]-. Accession No. CP000948.1, Escherichia coli uracil-DNA-glycosylase; [cited 2022 Nov. 14]. Webpage for: ncbi.nlm.nih.gov/nuccore/CP000948.1?from=2806541&to=2807230
Pezza, J. A., Kucera, R., & Sun, L. Polymerase fidelity: What is it, and what does it mean for your PCR?Webpage for: neb.com/tools-and-resources/feature-articles/polymerase-fidelity-what-is-it-and-what-does-it-mean-for-your-pcr Rockefeller University. Preparation of electro-competent DH10B cells. Laboratory of Molecular Parasitology. Webpage for: tryps.rockefeller.edu/Protocols/ecoli_electrocompetent.pdf
Roberts, R. J., Vincze, T., Posfai, J., & Macelis, D. (2023). REBASE: a database for DNA restriction and modification: enzymes, genes and genomes. Nucleic Acids Research. 51: D629-D630. doi: 10.1093/nar/gkac975 Sigma-Aldrich. Oligonucleotide quantification. Sigma-Aldrich.
Simirenko, L., Cheng, J. F., & Blaby, I. gRNASeqRET, a universal gRNA design tool. manuscript in prep Wang, G., Zhao, Z., Ke, J., Engel, Y., Shi, Y. M., Robinson, D., Bingol, K., Zhang, Z., Bowen, B., Louie, K., Wang, B., Evans, R., Miyamoto, Y., Cheng, K., Kosina, S., De Raad, M., Silva, L., Luhrs, A., Lubbe, A., . . . Yoshikuni Y. (2019). CRAGE enables rapid activation of biosynthetic gene clusters in undomesticated bacteria. Nature Microbiology, 4(12):2498-2510. doi: 10.1038/s41564-019-0573-8.
Wang, Y., Schellenberg, H., Walhorn, V., Toensing, K., & Anselmetti, D. (2017). Binding mechanism of PicoGreen to DNA characterized by magnetic tweezers and fluorescence spectroscopy. European Biophysics Journal, 46(6):561-566. doi: 10.1007/s00249-017-1204-z.
Warner, H. R., Duncan, B. K., Garrett, C., & Neuhard, J. (1981). Synthesis and metabolism of uracil-containing deoxyribonucleic acid in Escherichia coli. Journal of Bacteriology, 145(2):687-95. doi: 10.1128/jb.145.2.687-695.1981 Webgenetics. Plot GC content of DNA sequences.
Weitzman, M. (2018) Evaluation of Omega Mag-Bind@TotalPure NGS Beads for DNA Size Selection. University of Oregon. Webpage for: gc3fstorage.uoregon.edu/IMAGES/Evaluation_of_Omega_Mag-Bind_TotalPure_NGS_Beads_MWeitzman_April2018.pdf

Wong, K. K., Markillie, L. M., & Saffer J. D. (1997). A novel method for producing partial restriction digestion of DNA fragments by PCR with 5-methyl-CTP. Nucleic Acids Research, 25(20):4169-71. doi: 10.1093/nar/25.20.4169.
Wong, K. K., & McClelland, M. (1991). PCR with 5-methyl-dCTP replacing dCTP. Nucleic Acids Research, 19(5):1081-5. doi: 10.1093/nar/19.5.1081.
Ye, J., Coulouris, G., Zaretskaya, I., Cutcutache, I., Rozen, S., & Madden, T. L. (2012). Primer-BLAST: a tool to design target-specific primers for polymerase chain reaction. BMC bioinformatics, 13, 134

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Golden Gate Cloning Regardless of Internal Restriction Sites

Golden Gate cloning is a method of DNA assembly which uses Type IIS restriction enzymes to assemble DNA parts in a directional, scarless manner mostly independent of sequence context. It is especially useful for library and combinatorial DNA assembly, or the assembly of many parts. Golden Gate cloning is problematic, or impossible, when the chosen Type IIS enzyme has recognition sites internal to any of the parts being assembled, as internal sites can prevent parts from staying together through redigestion or may misligate on their overhangs to the wrong parts.

### 1.2 Materials and Methods

1.2.1 Nucleotide analogs. Nucleotides were purchased from New England Biolabs or Trilink, and if required individually diluted in nuclease-free water to 5 mM to standardize concentrations.

TABLE 1.1
List of nucleotides

| | |
|---|---|
| Thymine analogs | dTTP, dUTP, 4-Thio-dTTP, 5-Hydroxymethyl-dUTP |
| Adenosine analogs | dATP, N⁶methyl-dATP, 7-deaza-dATP |
| Cytosine analogs | dCTP, N⁴methyl-dCTP, 5-methyl-dCTP |
| Guanosine analogs | dGTP, 7-deaza-dGTP |

1.2.2 Oligos for Example 1. A list of oligos used in this chapter. Uppercase indicates template specific sequence, lowercase indicates non-template tails. Oligos were typically ordered from ThermoFisher with standard desalting.

TABLE 1.2
List of oligos for Example 1.

| | |
|---|---|
| R6k_2L_Sma-LguI-F | ttgctcttctCCATGGCTAATTCCCATGTCAGC (SEQ ID |
| | NO: 3) |
| R6k_2L_Sma-Esp3I-R | ttcgtctcttgGCAAAGCCTCGCAATCCAGT (SEQ ID NO: 4) |
| 346_6-adpt-F | TGCCACCTGACGTCTAAGAAACCA (SEQ ID NO: 5) |
| 346_6-4bp-adpt-R | GGCACTTTTCGGGGAAATGTGC (SEQ ID NO: 6) |
| R6k_2L_Sma-BbsI-F | ttgaagacCCCCTATAGTGAGTCGTATTAATTTCGC (SEQ |
| | ID NO: 7) |
| R6k_2L_Sma-BbsI-R | ttgaagacTATAGGGGAATTGTGAGCGGATAAC (SEQ ID |
| | NO: 8) |
| R6k_2L_Sma-Esp3I-Fv2 | ttcgtctctCCTATAGTGAGTCGTATTAATTTCGCCC (SEQ |
| | ID NO: 9) |
| R6k_2L_Sma-Esp3I-Rv2 | ttcgtctctTAGGGGAATTGTGAGCGGATAAC (SEQ ID |
| | NO: 10) |
| R6k_2L_Sma-LguI-Fv2 | ttgctcttctCCTATAGTGAGTCGTATTAATTTCGCCC (SEQ |
| | ID NO: 11) |
| R6k_2L_Sma-LguI-Rv2 | ttgctcttcTAGGGGAATTGTGAGCGGATAAC (SEQ ID |
| | NO: 12) |
| R6k_2L_Sma-EarI-Fv2 | ttctcttctCCTATAGTGAGTCGTATTAATTTCGCCC (SEQ |
| | ID NO: 13) |
| R6k_2L_Sma-EarI-Rv2 | ttctcttcTAGGGGAATTGTGAGCGGATAAC (SEQ ID |
| | NO: 14) |
| R6k_2L_Sma-BsaI-F | ttggtctctCCTATAGTGAGTCGTATTAATTTCGCCC (SEQ |
| | ID NO: 15) |
| R6k_2L_Sma-BsaI-R | ttggtctctTAGGGGAATTGTGAGCGGATAAC (SEQ ID |
| | NO: 16) |
| 346_6-PaqCI-F | ttcacctgcAGTGCCACCTGACGTCTAAGAAACCA (SEQ ID |
| | NO: 17) |
| 346_6-PaqCI-R | ttcacctgcTCAGGTGGCACTTTTCGGGGAAATG (SEQ ID |
| | NO: 18) |
| 346_6-3bp-adpt-R | GCACTTTTCGGGGAAATGTGCG (SEQ ID NO: 19) |
| 346_6-BsaI-F | ttggtctctCCACCTGACGTCTAAGAAACCA (SEQ ID |
| | NO: 20) |
| 346_6-BsaI-R | ttggtctctGTGGCACTTTTCGGGGAAATG (SEQ ID NO: 21) |
| 346_6-Esp3I-F | ttcgtctctCCACCTGACGTCTAAGAAACCA (SEQ ID NO: 22) |
| 346_6-Esp3I-R | ttcgtctctGTGGCACTTTTCGGGGAAATG (SEQ ID NO: 23) |
| CB_NT_insert-F | CCGAAAAGTGCCACCTGACGTCTAAGA (SEQ ID NO: 24) |
| CB_NT_insert-R | GATAACCGTATTACCGCCTTTGAGTGAGCT (SEQ ID |
| | NO: 25) |

1.2.3 PCR amplification of a 2033 bp target with regular dNTPs, dUTP, 4mCTP, 5mCTP, and 7dGTP. The primer pair R6k_2L_Sma-LguI-F and R6k_2L_Sma-Esp3I-R (Table 1.2) was used to amplify a 51% GC, 2033 bp region from the plasmid pR6k-2L-SmaI-Y.

PCR reactions were set up as follows: 1 ng of template DNA; 0.2 mM each nucleotide for all enzymes except Kapa HiFi which had 0.3 mM each nucleotide; 0.5 μM of each primer for Q5, Q5U, Phusion, SuperFi II, Phire and GoTaq G2 polymerases; 0.3 μM of each primer for the Smobio and Kapa polymerases; the recommended units of each polymerase; the recommended buffer for each polymerase; 1.5 mM MgSO₄ for Smo HiFi, 1 mM additional MgCl₂ for Kapa HiFi, and 2 mM MgCl₂ for GoTaq G2. Thermocycling consisted of: initial denaturing temperatures of 94° C. for 2 minutes for Smo HiFi, 95° C. for 2 minutes for GoTaq G2, 95° C. for 3 minutes for Kapa HiFi, and 98° C. for 2 minutes for the remaining enzymes; 30 cycles with denaturing temperatures of 94° C. for 30 seconds for Smo HiFi, 95° C. for 30 seconds for GoTaq G2, 98° C. for 20 seconds for Kapa HiFi, and 98° C. for 10 seconds for the remaining enzymes; and combined annealing/extensions of 62° C. for 5 minutes (8 minutes for GoTaq G2).

1.2.4 PCR amplification of a 4559 bp target with dUTP, 4mCTP, 5mCTP, and 7dGTP. The primer pair 346_6-adpt-F and 346_6-4 bp-adpt-R (Table 1.2) was used in PCR of a 51% GC, 4559 bp region from the plasmid 346-6-GG-mod.

PCR master mixes were set up as above for 50 μL reaction for Q5U, Q5, Q5 using the GC enhancer at 1×(Q5-GC), Phusion using the GC buffer (PG), Phusion using the HF buffer and Q5's GC enhancer at 1×(PH-GC), Kapa using primers at 0.5 μM final concentration, and SuperFi II. Nucleotides were added as above, as appropriate, after dispensing the master mixes. The dNTP, dUTP, and 7dGTP reactions were cycled at 98° C. for 30 seconds initial denaturation; 30 cycles of 98° C. for 10 seconds, 68° C. for 30 seconds, 72° C. for 4.5 minutes; 72° C. for 2 minutes following the cycling; and a hold at 4° C. The 4mCTP and 5mCTP reactions were cycled at 98° C. for 30 seconds initial denaturation; 30 cycles of 100° C. for 30 seconds (Wong, McClelland, 1991), 68° C. for 30 seconds, 72° C. for 4.5 minutes; 72° C. for 2 minutes following the cycling; and a hold at 4° C. Duplicate 7dGTP PCR reactions were performed at 98° C. for 30 seconds initial denaturation; 30 cycles of 98° C. for 10 seconds and 65° C. for 7 minutes; 65° C. for 5 minutes; and a hold at 4° C.

The 4mCTP PCR was attempted again under alternative cycling conditions. PCR reactions were set up as above using Q5, Phusion, SuperFi II, and Kapa HiFi. The cycling was 98° C. for 1 minute; 30 cycles of 98° C. for 15 seconds, 65° C. for 30 seconds, and 72° C. for 4.5 minutes; 72° C. for 4 minutes final extension; and hold at 4° C.

1.2.5 Comparison of Q5U to Phusion U for dUTP amplification. PCR reactions were set up in 50 μL with 1 unit of Q5U or Phusion U polymerases, 0.2 mM each dNTP (dUTP substituting for dTTP), 0.5 μM forward and reverse primers, and 1 ng of template DNA. Reactions were cycled at 98° C. for 30 seconds; 30 cycles of 98° C. for 10 seconds, 68° C. or 72° C. for 30 seconds, 72° C. for 4.5 minutes; followed by a final extension of 72° C. for 3 minutes; and a hold at 4° C.

An approximately 5.08 kb region from pR6k-2L-Sma-Y was a PCR target using an equimolar primer mix of the R6k_2L_Sma-BbsI-F and R, R6k_2L_Sma-Esp3I-Fv2 and Rv2, R6k_2L_Sma-LguI-Fv2 and Rv2, R6k_2L_Sma-EarI-Fv2 and Rv2, and R6k_2L_Sma-BsaI-F, and R oligos (Table 1.2). And an approximately 4.57 kb region from 346-6-GG-mod was a PCR target in five reactions using 346_6-PaqCI-F and R, 346_6-BsaI-F and R, 346_6-Esp3I-F and R, and 346_6-adpt-F and 346_6-3 bp-adpt-R (Table 1.2).

1.2.6 PCR of a 2731 bp target with dUTP, 7dGTP, 5mCTP, 7dATP, 6mATP, 4tdTTP, and 5homUTP. Reactions and control reactions were set up as follows: Q5U was used for dUTP PCR. Q5 was used for 7dG PCR. SuperFi II was used for 5mCTP PCR. Kapa HiFi, Phusion with the HF buffer, Q5, and SuperFi II were used for PCR with 7dATP and 6mATP. Fifty microliter reactions were set up with 1 unit of enzyme, 1 ng of plasmid DNA which was pre-linearized with AclI, 0.5 μM primers, and 0.2 mM of each nucleotide (0.3 mM for Kapa HiFi). The reactions were cycled at 98° C. for 30 seconds; 30 cycles of 98° C. for 10 seconds (100° C. for 30 seconds for 5mCTP), 68° C. for 20 seconds, and 72° C. for 3 minutes; followed by a final extension of 72° C. for 2 minutes; and a hold at 4° C.

PCR was reattempted using Q5U and Phusion U polymerases with the dTTP and dATP analogs 7dATP, 6mATP, 4tdTTP, and 5homUTP. Reactions were set up as above, and cycled at 98° C. for 30 seconds; 25 cycles of 98° C. for 10 seconds, 70° C. for 30 seconds, and 72° C. for 3 minutes; followed by a final extension of 72° C. for 5 minutes; and a hold at 4° C.

The PCR target was a 2731 bp region from 346-6-GG-mod using the CB_NT_insert-F and R primers (Table 1.2).

1.2.7 Sequencing of dNTP, dUTP, 5mCTP, and 7dGTP amplified targets. Full-length amplicons were submitted without shearing for sequencing on a Pacific Biosciences Sequel IIe, with results visualized using the Broad Institute's Integrated Genomics Viewer (IGV) (Appendix A).

### 1.3 Results

1.3.1 Polymerase comparison. In order to test the ability of some high-fidelity polymerases to amplify DNA with alternative nucleotides, a total of nine polymerases available in the lab were tested. Some of these polymerases were known to be able to amplify with some of the alternative nucleotides, some were previously known to not be able to amplify with some of the alternative nucleotides. Polymerases tested were Smobio's Smo HiFi (with and without 10% DMSO) and Smo G-HiFi, NEB's Q5 Hot Start and Q5U HotStart, Thermo's Phusion Hot Start II and Platinum SuperFi II, Roche's Kapa HiFi, with Promega's GoTaq G2 Flexi and Thermo's Phire Hot Start II polymerases as lower fidelity controls.

Reaction mixes and cycling conditions were based on the recommended conditions for each enzyme, but the cycling conditions were slightly modified to allow combining reactions on the limited number of thermocyclers. A combined 2-step annealing and extension step at the low temperature of 62° C. was chosen due to the known effect of 4mCTP on decreasing DNA melting temperature; it was assumed this would be the most permissive cycling condition.

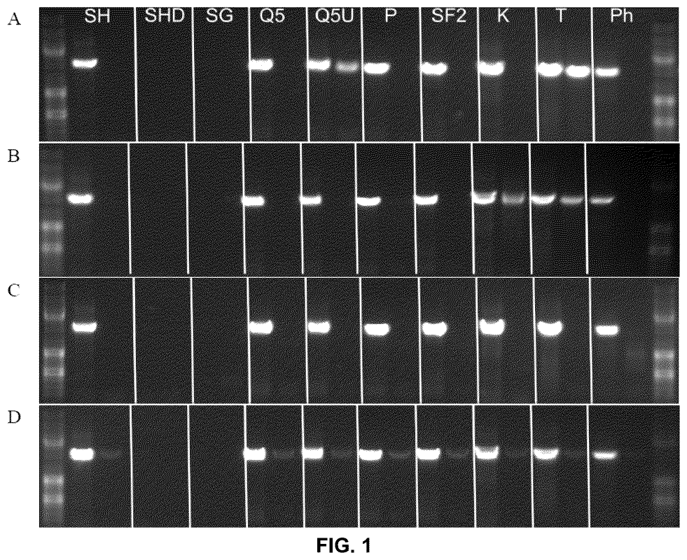

The results in FIG. 1 demonstrate that, as expected, only Q5U and Taq could amplify DNA with dUTP, though with noticeably lower band intensities. All of the polymerases which amplified using dNTPs could also amplify with 7dGTP, though fluorescent quenching resulted in faint bands. And only Kapa HiFi and Taq could amplify DNA with 4mCTP. Further testing with Smo HiFi, Taq, and Phire was abandoned.

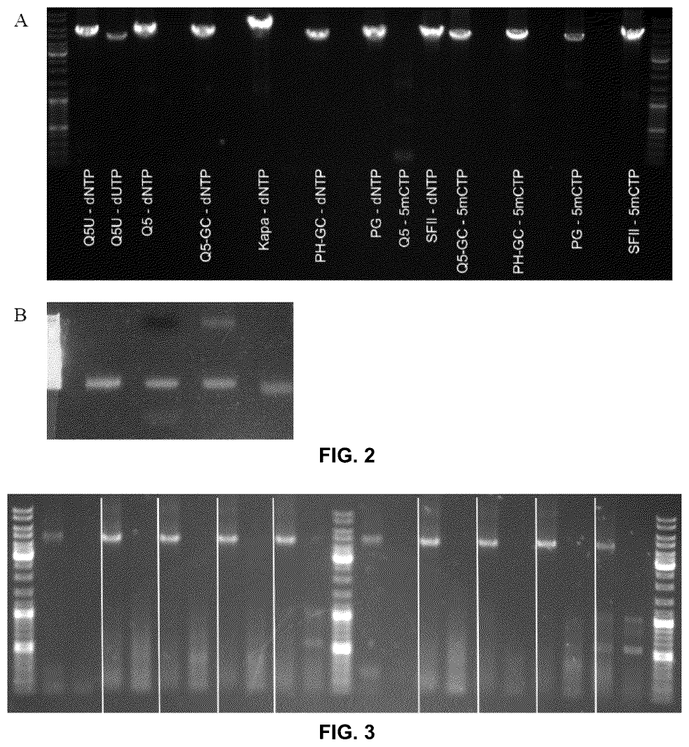

A longer 4559 bp target was amplified consisting of the entire 346-6-GG-mod vector sequence, which allowed recirculation of the vector in chapter 2. In FIG. 2 amplification was shown to mostly fail for 4mCTP at higher denaturing, annealing, and extension temperatures, though very faint amplification was seen with SuperFi II (FIG. 2, panel B) when the annealing temperature was 65° C. for 30 seconds. These higher temperatures, along with additives, allowed amplification with 5mCTP. Higher annealing and extension temperatures retained amplification with dUTP (FIG. 2, panel A) and 7dGTP.

Q5U was compared to Phusion U for the amplification of the entirety of the pR6k-2L-SmaI-Y and 346-6-GG-mod vectors using dUTP at the calculated Q5U annealing temperatures of the 346-6-GG-mod target (NEB, web). While Phusion U showed some non-specific bands, amplification with Q5U showed the expected sized amplicon for all reactions (FIG. 3).

From these results Q5U was chosen for non-test amplification using dUTP, and Q5 and SuperFi II were chosen for non-test amplification using other nucleotides.

The ladder is Thermo's GeneRuler DNA mix. The 7dGTP Crystal Violet gel was not imaged, but showed that Q5 and SuperFi II amplified well at 72° C., though SuperFi II had a lower non-specific band, and 65° C. 2-step PCR Q5 with GC enhancer and SuperFi II both amplified well, while the regular Q5 reaction evaporated on the cycler.

A) The 4mCTP reactions all failed and are not indicated on the gel. The other reactions are labeled by the polymerase (P=Phusion, SFII=SuperFi II), by any variable buffers or additives (H=Phusion HF buffer, G=Phusion GC buffer, GC=Q5's GC enhancer at 1× concentration), and by the nucleotide mix.

B) Q5, Phusion, SuperFi II, Kapa 4mCTP PCR reactions with 1 μg of ladder. Exposure time was extended to visualize anything.

Ladder is the Thermo GeneRuler DNA mix. The left set of reactions are at a 68° C. annealing temperature, the right set at a 72° C. annealing temperature. For each set of two reactions the left lane uses Q5U and the right lane is Phusion U. From left to right the reactions are the R6k mix, 346_6-PaqCI, 346_6-BsaI, 346_6-Esp3I, and 346_6-3 bp-adpt.

1.3.2 Nucleotide comparisons. 7-deaza-dGTP (7dGTP) (Grime, et al., 1991), N⁴methyl-dCTP (4mCTP) (Flores-Juárez, et al., 2016), and 5-methyl-dCTP (5mCTP) (Wong, et al., 1997) were chosen based on previously published papers regarding their use in PCR and their effect on the inhibition of restriction digestion. Deoxyuridine (dUTP) was chosen to use with high-fidelity DNA polymerases specifically engineered to read and write it as a dTTP analog. N⁶methyl-dATP (6mATP) was chosen as this is a common natural DNA modification that does not appear to inhibit amplification when present in the template; 7-deaza-dATP (7dATP) was chosen due to similarity to 7dGTP; and the other two thymine analogs, 4-Thio-dTTP (4tdTTP) and 5-Hydroxymethyl-dUTP (5homUTP), were chosen due to the minimal nature of their modifications.

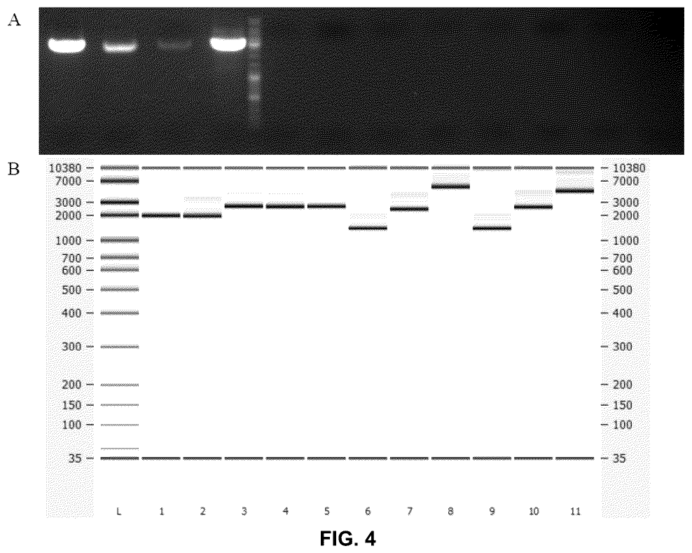

As shown above successful amplification was achieved with dUTP, 4mCTP, 5mCTP, and 7dGTP. Amplification of a 2731 bp target from 346-6-GG-mod was attempted with dNTPs and the previously validated dUTP, 7dGTP, and 5mCTP to compare with the two adenosine and two additional thymine analogs. Under the tested conditions, all amplifications with the adenosine (FIG. 4, panel A) and additional thymine analogs failed. Table 1.3 summarizes the results.

The dNTP, dUTP, 7dGTP, and 5mCTP reactions were appropriately diluted and run out on an Agilent 2100 Bioanalyzer using a High Sensitivity DNA chip (FIG. 4, panel B). On an agarose gel the dUTP, 7dGTP, and 5mCTP amplified bands migrate at about the same speed as the dNTP band, while on the Bioanalyzer the dUTP amplicon migrates faster than the 7dGTP amplicon, which migrates almost the same as the dNTP amplicon, which migrates faster than the 5mCTP amplicon.

To determine that gross amplification errors were not introduced by PCR with the alternative nucleotides, dUTP amplicons from the Q5U versus Phusion U comparison, along with identical dNTPs, 5mCTP, and 7dGTP amplicons were submitted for sequencing using a Pacific Biosciences Sequel IIe (Appendix A). The dUTP amplicon had no coverage, and reads of the 7dGTP amplicon were sheared and low coverage.

To better identify specific fidelity, sequencing of individual clones took place in chapters 2 and 4.

TABLE 1.3
PCR amplification with alternate nucleotides summary.

| | dNTP | 4mC* | 5mC* | 7dG* | dU | 4tT | 5homU | 6mA | 7dA |
|---|---|---|---|---|---|---|---|---|---|
| Smo HiFi | + | − | NA | + | − | NA | NA | NA | NA |
| Q5 | + | − | +* | + | − | NA | NA | − | − |
| Phusion | + | − | +* | + | − | NA | NA | − | − |
| SuperFi II | + | P | + | + | − | NA | NA | − | − |
| Kapa HiFi | + | OK | NA | + | − | NA | NA | − | − |
| GoTaq G2 | + | OK | NA | + | + | NA | NA | NA | NA |
| Phire | + | − | NA | + | − | NA | NA | NA | NA |
| Q5U | + | − | NA | + | OK | − | − | − | − |
| Phusion U | NA | NA | NA | NA | VP | − | VP | − | − |

*N⁴methyl-dCTP—required low temperature 2-step protocol to amplify.
*5-methyl-dCTP—requires elevated denaturing temp to amplify; Q5 required GC enhancer, Phusion has higher yields with Q5's GC enhancer.
*7-deaza-dGTP—quenches fluorescence, requires nanodrop to quantify (Dudová, Spacek, Havran, et al., 2016).

### 1.4 Discussion

I have tested 8 high-fidelity polymerases and found that some can consistently amplify DNA while fully substituting dUTP for dTTP, 5mCTP for dCTP, and 7dGTP for dGTP, though optimization may be required. 4mCTP is another candidate, but further testing would be required for consistent results with 4mCTP PCR. Additionally, the one high-fidelity enzyme that showed good amplification with 4mCTP under the tested conditions, Kapa HiFi, has among the lowest fidelity of the very high-fidelity enzymes (Hadigol and Khiabanian, 2018). Kapa HiFi may be fine to use for PCR of short pieces of DNA for assembly, but if tens of kilobases need to be modified, it may be more efficient trying another approach with one of the other nucleotides.

At the time of these experiments, SuperFi II and Kapa HiFi uracil-tolerant polymerases were available, but only in master mix formats with regular dNTPs, so could not be tested for the purpose of fully substituting dUTP for dTTP.

A number of the commonly used Type IIS restriction enzymes lack adenosine on the top strand of their recognition sites (when reading from 5′ of the cut site), allowing primer-borne sites to remain fully unmodified when amplified with dUTP, while internal sites will be modified on the top strand. For these Type IIS enzymes which may have cleavage blocked or impaired when uracil substitutes for thymine, PCR amplification with dUTP is a promising candidate for restriction enzyme-based cloning. For 5mCTP amplified DNA, fewer Type IIS sites lack guanosine on the top strand of their recognition site, though some do, and for those that have some guanosines on the top strand REBASE (Roberts, et al., 2023) shows that methylation at various bases doesn't always block or impair digestion, so amplification with 5mCTP may still be useful for cloning purposes. None of the common Type IIS enzymes completely lack cytosine on the top strand of their recognition site, though incorporation of 7-deaza-guanosine into recognition sites does not completely block all restriction enzymes from digesting, so 7dGTP amplified DNA is still worth investigating.

Q5U PCR with dUTP showed decreased yields compared to the other nucleotides in my hands. Larger reaction volumes may be required for sufficient yield for cloning purposes. It is possible that high-GC amplicons will show less decrease in yield as there are fewer uracil incorporation events. The use of uracil in DNA may require special cloning strains (chapter 3). On a bioanalyzer chip it runs significantly faster than dTTP amplified DNA, which should be considered if using microfluidic electrophoresis for PCR QC. Finally, direct sequencing of dUTP amplicons, prior to cloning, requires sequencing chemistry and polymerases that can read uracil in DNA. Given the ability of Taq polymerase to read and write uracils conventional Sanger sequencing should work, but Pacific Biosciences sequencing does not, which would require another round of PCR with dTTP to generate template for sequencing.

PCR with 5mCTP required higher and longer denaturing steps, and benefited from additives such as the Q5 GC enhancer. 5mCTP may be problematic to use when amplifying higher-GC content amplicons, and may be better for low-GC amplicons. Also, its use results in hyper-methylation, which may have unanticipated effects when transformed into bacteria. On a bioanalyzer chip it ran significantly slower than dCTP amplified DNA, indicating, with the dUTP results, a qualitative difference between conventional agarose gel electrophoresis and microfluidics electrophoresis. Some thermocyclers are limited to less than 100° C. If a 100° C. denaturing temperature cannot be used, it may be necessary to experiment with DMSO or other additives to get robust, specific amplification with 5mCTP.

PCR with 7dGTP generates bands that are barely visible on a gel. 7-deaza-guanosine substituted DNA is impossible to quantify with fluorescence, at least without significant theoretical and practical work. While E. coli's native polymerase has no problem amplifying 7-deaza-guanosine containing DNA, its effect in vivo is unknown to me. On a bioanalyzer chip it runs slightly faster than dGTP amplified DNA. The sheared reads in the Pacific Biosciences Sequel IIe 7dGTP sequence analysis is interesting and unfortunate. This may be an artifact of the Pacific Biosciences Sequel IIe library generation process, or selection bias by the Sequel IIe.

While in these experiments I fully substituted the various nucleotides, for the above reasons it may be worthwhile only partially substituting nucleotides in PCR. While not all recognition sites for a particular enzyme will be blocked in this approach, a post-PCR digestion, followed by size-selection purification, such as an agarose gel, would result in full-length PCR products with only partial nucleotide substitution.

If additional modified nucleotides will be investigated for PCR it is important to validate their mutagenic potential, both in PCR, and in vivo. For example, the adenosine analog inosine is known to base pair with all other nucleosides, and is thus used for random mutagenesis in PCR (Kuipers, 1996). Despite the ability of Q5U to amplify with dITP, this is the reason I avoided testing it. Other nucleotides may have reactive modifications with unknown mutagenic effect in vivo. This extends even to the successful nucleotide modifications used in this chapter. If PCR with modified nucleotides is performed for cloning purposes it will be necessary to ensure that the nucleotides do not greatly decrease PCR fidelity, or have negative effects on replication fidelity in vivo.

For use with cloning it is important to accurately quantify DNA, especially if several pieces will be assembled together. For fluorescence quantification methods it is necessary to understand the effect of modified nucleotides on fluorescence emission or quenching (Dudová, Špaček, Havran, et al., 2016). Fluorescent agents have multiple methods of binding to double stranded DNA (Dragan, et al., 2010) (Wang, et al., 2017), so any possible interference in any of the mechanisms of binding may have an effect on quantification. If spectrophotometric methods are used, potential contaminants with overlapping absorption spectra need to be considered, and possibly eliminated with stringent purification and washing procedures. At least as important are the proportions of individual nucleotides. The extinction coefficients of nucleotides range over 2-fold, from a low with cytosine to a high with adenine (Sigma-Aldrich, web). While spectrophotometric quantification of DNA sequences with similar AT/GC content will at least be proportionately accurate, quantification of DNA sequences with greatly different AT/GC content would require specific calculations based on the nucleotide content of each sequence. The addition of modified nucleotides will require calculations which consider the extinction coefficients for these modified nucleotides. Without special software, these calculations may be even more difficult if only partial nucleotide substitution is used.

While it is likely possible to amplify DNA with more than one modified nucleotide, for the purposes of cloning this is probably overkill. It is sufficient that three of the four natural nucleotides can be substituted with modified nucleotides that block the commonly used Type IIS restriction enzymes (Chapter 2).

### Example 2

### The Effect of Chemically Modified Nucleotides on Restriction Digestion and Molecular Cloning

### 2.1 Introduction

In chapter 1, I have shown that a subset of high-fidelity DNA polymerases and some specific modified nucleotide analogs can generate sufficient quantities of fully substituted DNA products. In this chapter I test if these PCR products can allow the silencing of fully modified internal restriction sites while either leaving the flanking restrictions sites sufficiently unmodified to allow digestion, or allowing ligation of unmodified adapters and subsequent digestion from these adapters.

Primer-borne restriction sites are the most straight-forward to clone with, but the addition of tails to primers can lead to amplification failure due to non-specific hybridization or secondary structure. Additionally, primer-borne restriction sites will only work with restriction enzymes that are not blocked by modification on the synthesized strand. Ligation of unmodified adapters allows PCR with completely specific, non-tailed primers, or addition of adapters to non-amplified linear DNA, such as through Type IIP restriction-digestion freed and sequence verified clones, which is modified with methyltransferases to block internal restriction sites. Additionally, unmodified adapters ligated to modified DNA theoretically allowing cloning with all restriction enzymes.

Clonability of modified DNA is another consideration. While most E. coli strains naturally methylate cytosine residues in certain sequence contexts, hypermethylation of nucleotides is known to condense DNA in eukaryotes. Various other modifications are seen as DNA damage and excised (Hayakawa, et al, 1978), which would be problematic for successful cloning of fully substituted DNA. So in order to use these DNA modifications for cloning purposes it's necessary to not only check if they facilitate targeted restriction digestion and ligation, but also verify that E. coli can replicate the modified DNA.

This strategy is inspired by the MASTER ligation technique (Chen, et al., 2013). The MASTER ligation technique uses MspJI, a type IIM/S enzyme which minimally requires hemi-methylation of its recognition site in order to cut DNA. The hemi-methylated recognition site is introduced to flank a non-methylated PCR product through sequential PCR, first with forward and reverse tailed primers, and then with a methylated primer specific for the tails. Alternately the insert of interest can be digested out of unmethylated DNA and ligated to methylated adapters. This allows scarless assembly of DNA elements that contain internal restriction enzyme recognition sites. Drawbacks of the MASTER ligation technique include the requirement for sequential PCR, with GC-rich 20 nt primer tails on the original oligos, or unmethylated DNA that lack particular internal restriction enzymes which are used for digestion and ligation to adapters. The digestion and ligation take place sequentially, instead of simultaneously, requiring a two-pot Type IIS approach. MspJI can exhibit start activity on non-methylated DNA, so stoichiometric optimization of enzyme, activating oligo, DNA, and digestion time is required for complete and specific digestion of parts.

The Type IIS restriction enzymes have been used for nearly thirty years for scarless assembly of DNA parts (Beck and Burtscher, 1994). Their popularity has increased with development of the Golden Gate (Engler, et al., 2008) family of cloning techniques. A noted feature of Type IIS enzymes is that they usually have non-palindromic recognition sites. This makes them ideal candidates for testing with the flanking restriction site PCR strategy mentioned above, as it is theoretically possible to pick modified nucleotides which do not impede digestion when present only on the complementary strand, but will block or impede digestion when present on the primer.

For enzymes in which this is not the case—where digestion would be blocked by modification of the complementary strand—it would be possible to ligate adapters containing fully-unmodified restriction enzyme recognition sites onto a modified PCR amplicon or unamplified linear DNA freed from sequence verified clones through restriction digestion. This would allow subsequent restriction digestion and freeing of single-stranded overhangs for ligation. If the enzyme is a Type IIS enzyme, the freed overhangs could be within the modified PCR amplicon, and thus allow a scarless assembly.

Targeted methylation has been used to block specific restriction enzyme sites while leaving other sites unblocked in hierarchical Type IIS cloning techniques (Lin and O'Callaghan, 2018) and Golden Gate-like type IIP cloning techniques (Matsumura, 2022). These techniques use restriction enzyme sites designed with or without overlapping methyltransferase target sites, and then either perform methylation of these sites through transformation into E. coli engineered to express the specific methyltransferases, or demethylated by transforming into E. coli not expressing the specific methyltransferases. Theoretically they can also be methylated in vitro with purified methyltransferase enzymes.

Although PCR can be used to methylate all cytosines but those in the primers, as shown in chapter 1, somewhat more targeted methylations can be performed by using commercially available methyltransferases. In this chapter I also test New England Biolabs's (NEB) CpG methyltransferase which methylates the C⁵ position of cytosines in CG dinucleotides, GpC methyltransferase which methylates the C⁵ position of cytosines in GC dinucleotides, and EcoGII methyltransferase which methylates the N⁶ position of most adenosines. The CpG and GpC methyltransferases will methylate some cytosines of some of the popular Type IIS restriction enzymes, and the EcoGII methyltransferase will methylate all adenosines of all of the popular Type IIS restriction enzymes. As in vitro methylation cannot target just internal restriction enzyme sites, but also primer-borne sites, it would not be possible to block internal sites and leave flanking sites cleavable. It is, however, possible to ligate adapters containing unmethylated restriction sites onto the methylated DNA for subsequent digestion and assembly.

Here I test the digestibility of methylated or otherwise modified DNA from primer-borne or adapter-ligated restriction enzyme sites showing which modification and cloning strategies that can be used with various of the Type IIS restriction enzymes. By assembling the modified DNA into unmodified plasmid vectors I test the effect of the DNA modifications on in vivo replication in E. coli. And through modification of the plasmid vector backbones themselves I test whether DNA modifications can be used to silence internal restriction enzyme sites in destination plasmids.

### 2.2 Materials and Methods

2.2.1 Restriction enzymes. The following Type IIS restriction enzymes are from ThermoFisher (AarI, BpiI, and LguI) and NEB (the rest). Table 2.1 contains the restriction enzyme names, recognition sites (cleavage points indicated with [symbol] ), and REBASE modification sensitivity URL (Roberts, et al., 2023). AarI and PaqCI are isoschizomers, as are BbsI-HF and BpiI, BsmBI and Esp3I, and LguI and SapI. BfuAI can cleave AarI/PaqCI sites, and EarI can cleave LguI/SapI sites, as well as additional sites.

TABLE 2.1
List of Type IIS restriction enzymes.

| | | |
|---|---|---|
| AarI | CACCTGCNNNN\| | http://rebase.neb.com/cgi- |
| | GTGGACGNNNNNNNN\| | bin msget?AarI |
| PaqCI | CACCTGCNNNN\| | http://rebase.neb.com/cgi- |
| | GTGGACGNNNNNNNN\| | bin/msget?PaqCI |
| BfuAI | ACCTGCNNNN\| | http://rebase.neb.com/cgi- |
| | TGGACGNNNNNNNN\| | bin/msget?BfuAI |
| BbsI- | GAAGACNN\| | http://rebase.neb.com/cgi- |
| HF | CTTCTGNNNNNN\| | bin/msget?BbsI |
| BpiI | GAAGACNN\| | http://rebase.neb.com/cgi- |
| | CTTCTGNNNNNN\| | bin/msget?BpiI |
| BsaI- | GGTCTCN\| | http://rebase.neb.com/cgi- |
| HFv2 | CCAGAGNNNNN\| | bin/msget?BsaI |
| BsmBI- | CGTCTCN\| | http://rebase.neb.com/cgi- |
| v2 | GCAGAGNNNNN\| | bin/msget?BsmBI |
| Esp3I | CGTCTCN\| | http://rebase.neb.com/cgi- |
| | GCAGAGNNNNN\| | bin/msget?Esp3I |
| LguI | GCTCTTCN\| | http:/rebase.neb.com/cgi- |
| | CGAGAAGNNNN\| | bin/msget?LguI |
| SapI | GCTCTTCN\| | http:/rebase.neb.com/cgi- |
| | CGAGAAGNNNN\| | bin/msget?SapI |
| EarI | CTCTTCN\| | http://rebase.neb.com/cgi- |
| | GAGAAGNNNN\| | bin/msget?EarI |

2.2.2 Oligos for Example 2. A list of oligos used in this chapter. Uppercase indicates template specific sequence, lowercase indicates non-template tails. Restriction enzyme sites are underlined. The Esp3J-TA and LguI-TA adapters were designed based on the MASTER adapter (Chen, et al., 2013), and validated for proper hairpin formation using IDT-DNA's OligoAnalyzer tool (IDT-DNA, web). Oligos were typically ordered from ThermoFisher or TDT-DNA with standard desalting.

TABLE 2.2
List of oligos for Example 2.

| | |
|---|---|
| pENTR_UniF_RC | GGGAAGGGTGGGCGCGCCGACCC (SEQ ID NO: 26) |
| pENTR_UniR_RC | GGGGAAGGGCTCCTTCTTAAAGTTAAACAAGGCGG (SEQ ID NO: 27) |
| CB_346-F | AGCTCACTCAAAGGCGGTAATACGGTTATC (SEQ ID NO: 28) |
| CB_346-R | GTTTCTTAGACGTCAGGTGGCACTTTTCG (SEQ ID NO: 29) |
| R6k-to-346-Esp3I-F | ttcgtctcttaatCGAGCGCTTCCGAAAATGCAACG (SEQ ID NO: 30) |
| R6k-to-346-Esp3I-R | ttcgtctcttcgaCGTTGTAAAACGACGGCCAGT (SEQ ID NO: 31) |
| R6k-to-346-BsaI-F | ttggtctcttaatCGAGCGCTTCCGAAAATGCAACG (SEQ ID NO: 32) |
| R6k-to-346-BsaI-R | ttggtctcttcgaCGTTGTAAAACGACGGCCAGT (SEQ ID NO: 33) |
| R6k-to-346-BbsI-F | aagaagacaataatCGAGCGCTTCCGAAAATGCAACG (SEQ ID NO: 34) |
| R6k-to-346-BbsI-R | aagaagacaatcgaCGTTGTAAAACGACGGCCAGT (SEQ ID NO: 35) |
| R6k-to-346-PaqCI-F | ttcacctgctttttaatCGAGCGCTTCCGAAAATGCAACG (SEQ ID NO: 36) |
| R6k-to-346-PaqCI-R | ttcacctgctttttcgaCGTTGTAAAACGACGGCCAGT (SEQ ID NO: 37) |
| R6k-to-346-adpt4-F | taatCGAGCGCTTCCGAAAATGCAACG (SEQ ID NO: 38) |
| R6k-to-346-adpt4-R | tcgaCGTTGTAAAACGACGGCCAGT (SEQ ID NO: 39) |
| R6k-to-346-adpt3-F | aggCGAGCGCTTCCGAAAATGCAACG (SEQ ID NO: 40) |
| R6k-to-346-adpt3-R | cctcgaCGTTGTAAAACGACGGCCAGT (SEQ ID NO: 41) |
| R6k-to-346-LguI-F | ttgctcttctaggCGAGCGCTTCCGAAAATGCAACG (SEQ ID NO: 42) |
| R6k-to-346-LguI-R | ttgctcttctcctcgaCGTTGTAAAACGACGGCCAGT (SEQ ID NO: 43) |
| Esp3I-TA | GAGACGGATCCCTTTTTTTAGGGATCCGTCTCT (SEQ ID NO: 44) |
| LguI-TA | GAAGAGCGATCCCTTTTTTTAGGGATCGCTCTTCT (SEQ ID NO: 45) |
| BamBgl.scr.F | AAAAGTGCCACCTGACGTCT (SEQ ID NO: 46) |
| TALE.scrR | GATCATTTGTCAGAGCGGCTAAAGCAGG (SEQ ID NO: 47) |
| TALE.scrF | TCAAGATAGCCAAAAGGGGAGGAGTGA (SEQ ID NO: 48) |
| BamBgl.scr.R | CCTTTTGCTGGCCTTTTGCT (SEQ ID NO: 49) |

2.2.3 Methylation of linearized or supercoiled plasmids. Typically, 50 μL or 100 μL reactions were set up with 1 μL of 32 mM S-adenosylmethionine (SAM); 2 μL of CpG methyltransferase (8 units), GpC methyltransferase (8 units), or EcoGII methyltransferase (10 units) in the appropriate buffer. Reactions were incubated for 4 hours at 37° C., 20 minutes at 65° C. (optional), and held at 4° C. prior to purification.

2.2.4 Purification of reactions. Methyltransferase and PCR reactions were either purified with commercial purification columns from Zymo Research using a homebrewed guanidine thiocyanate binding buffer, or using Omega Bio-tek Mag-Bind® TotalPure NGS beads. When using the beads between 0.5× and 1× reaction volume of beads was used per reaction depending on the size of the amplicon and for size-selecting away any visible non-specific bands (Weitzman, 2018).

2.2.5 Transformation into chemically competent cells. Typically, 1 μL to 5 μL of assembly reactions were added to 50 μL of pre-made CCMB80 Top10 or Top10 F′ cells. This was incubated on ice for 20 to 30 minutes, placed in a 42° C. water bath for 1 minute, placed back on ice, and then outgrown with 100 μL of SOC for 1 hour at 37° C. or at least 2 hours at 30° C.

2.2.6 Transformation into electrocompetent DH10β(Δung). Competent cells prepared in chapter 3 were diluted 1 part cells to 5 parts ice-cold water or 10% glycerol. Typically, 0.5 μL to 1 μL of assembly reactions were added to 40 μL aliquots of competent cells and electroporated using a 2 mm gap, 25-well or 96-well electroporation plate on a BTX ECM 630 exponential decay electroporation system with HT-200 plate handler. The setting was 2.4 kV, 200Ω, and 25 μF, and the cells were outgrown with 1 mL of SOC, typically for 2 hours at 30° C.

2.2.7 Restriction digestion of modified linear plasmid DNA. Reactions were run out on agarose gels or an Agilent 2100 Bioanalyzer with HS DNA chip to visualize. Ten microliter reactions were performed for 1 to 15 hours at the recommended digestion temperature of each enzyme. Reactions were typically stopped by addition of loading buffer with SDS, addition of proteinase K, or incubation at 80° C. for 20 minutes, in the latter two cases 5M betaine was used as a 6× loading buffer to eliminate dye shadows. Typically, between 0.5 μL and 1 μL of enzyme was used. PaqCI and BfuAI digests typically used PaqCI activator at 1:1 to 1:2 ratio, and AarI digests used the AarI oligo at 1× concentration.

2.2.8 Cloning of DNA modified across the entire vector. Linear plasmids were PCR amplified with dNTPs, dUTP, 5mCTP, and 7dGTP using the pENTR_UniF_RC and FC oligos (Table 2.2), or methylated as above with CpG, GpC, and EcoGII methyltransferase. Twenty or 50 nanograms of DNA was added to 5 units of T4 polynucleotide kinase and 1000 cohesive end units of T4 DNA ligase in 10 μL reactions with 1× StickTogether buffer. Reactions proceeded for 30 minutes at room temperature and were transformed into chemically competent Top10 cells.

Undigested 346-6-GG-mod plasmid DNA was methylated with CpG, GpC, and EcoGII methyltransferases. This undigested as well as linear methylated or PCR modified plasmid DNA from chapter 1 was added to 10 μL reactions containing 1×T4 DNA ligase buffer, 2 units of T4 polynucleotide kinase and 400 units of T4 DNA ligase. The reactions were incubated for 16 hours at 25° C., 10 minutes at 65° C., and held at 4° C. before transformation into DH10β(Δung) electrocompetent cells.

2.2.9 Chewback cloning of modified DNA into an unmodified vector. Chewback cloning refers to techniques which use exonuclease digestion of homologous ends of DNA parts to expose single-stranded, complementary overlaps that anneal and thus join the parts together. Examples are Gibson and In-Fusion assembly.

An 1884 bp region of 346-6-GG-mod which contains the pUC origin of replication and an AmpR gene was amplified from 1 ng of PstI-linearized vector DNA with primers CB_346-F and CB_346-R (Table 2.2) using Q5 and Thermo's SuperFi II polymerases. Cycling was 98° C. for 30 seconds; 25 cycles of 98° C. for 10 seconds, 68° C. for 20 seconds, and 72° C. for 2 minutes; followed by a final extension at 72° C. for 2 minutes; and a hold at 4° C. Amplicons were gel purified.

NEBuilder HiFi and Clontech In-Fusion reactions were set up with 5 fmoles of the gel purified 346-6-GG-mod vector backbone amplicon and 10 fmoles of the 346-6-GG-mod CB_NT-insert inserts PCR modified in chapter 1 section 1.3.6, and methylated in this chapter. Four microliter reactions consisting of 1×NEBuilder HiFi were performed for 1 hour at 50° C. and then frozen overnight, and 4 μL InFusion reactions were performed for 15 minutes at 50° C. and then frozen overnight. The reactions were transformed into Top10 F′ cells.

2.2.10 Golden Gate cloning. 2.2.10.1 Vector and insert prep. The vector 346-6-GG-mod was linearized with either PaqCI or LguI and dephosphorylated with QuickCIP. The LguI digest was treated with GpC methyltransferase and used for the LguI and EarI assemblies.

The R6k-to-346 oligos (Table 2.2) were used to amplify a 1645 bp (+tails) target from the plasmid pR6k-2L-SmaI-Y. Q5 was used for the dNTP and 7dGTP PCRs, Q5U was used for the dUTP PCRs, and SuperFi II was used for the 5mCTP PCRs. The 5mCTP reactions were cycled at 98° C. for 30 seconds; 30 cycles of 98° C. for 30 seconds, 68° C. for 30 seconds, 72° C. for 3 minutes; followed by a final extension for 5 minutes at 72° C.; and a hold at 4° C. The other reactions were cycled for 98° C. for 30 seconds; 30 cycles of 98° C. for 10 seconds, 68° C. for 15 seconds, 72° C. for 2 minutes; followed by a final extension for 5 minutes at 72° C.; and a hold at 4° C. Reactions bead purified.

1.3 μg to 1.6 μg of various of the R6k dNTP amplicons were methylated with CpG, GpC, or EcoGII methyltransferase, and then purified.

The Adpt4 and Adpt3 inserts were A-tailed using NEB's NEBNext dA-Tailing Module. Approximately 1.4 μg of each DNA was incubated for 30 minutes at 37° C. in 50 μL reaction volumes with 3 μL of Klenow (exo-) following NEB's A-tailing protocol, and then column purified.

The Esp3I-TA and LguI-TA oligos in Table 2.2 were resuspended in a buffer of 100 mM NaCl, 9.8 mM Tris-HCl pH 8, and 98 μM EDTA. These were self-annealed with a 2 minute incubation at 94° C., followed by a drop to 4° C. by 0.5° C. every 10 seconds. 300 pmoles of each adapter was added to a 50 μL reaction with 1×T4 DNA ligase buffer and 10 units of NEB's T4 polynucleotide kinase. The reactions were incubated for 30 minutes at 37° C. and then placed on ice.

Approximately 1 μg (8 μL) of A-tailed DNA was ligated to 12 pmoles (2 μL) of the LguI-TA (Adpt3) or the Esp3I-TA (Adpt4) phosphorylated adapters using 10 μL of NEB's 2×Blunt/TA Ligase Master Mix (NEB, 2018) in a total volume of 20 μL for 1 hour at room temperature, and then bead purified.

The BbsI, BsaI, and portions of the LguI primer set amplicons were digested overnight at 37° C. with 1 μL of, respectively, BbsI-HF, BsaI-HFv2, and EarI. Reactions were then bead purified. All DNA was quantified by nanodrop.

2.2.10.2 Golden Gate reactions transformed into Top10. Each 10 μL Golden Gate reaction used ˜13.6 fmoles of vector (38.2 ng) and ˜27.2 fmoles of insert (27.9 ng) and 1×T4 DNA Ligase buffer. BsmBI-v2 reactions used 1 μL of NEB's NEBridge® Golden Gate Enzyme Mix (BsmBI-v2). The other reactions used 200 units of T4 DNA ligase and 5 units of PaqCI (0.25 μL adapter) or Esp3I, or 0.5 μL FastDigest LguI.

Cycling was 30 cycles of 37° C. for 5 minutes (42° C. for 1 minute for BsmBI-v2) and 16° C. for 5 minutes (1 minute for BsmBI-v2), followed by 50° C. for 5 minutes, 65° C. for 10 minutes, and a hold at 4° C.

2.2.10.3 Ligation reactions. The BbsI, BsaI, and EarI digested inserts were ligated into the vector using the same amounts and molar ratios of vectors and inserts as for the Golden Gate reactions. Each 10 μL ligation was performed with 1× StickTogether buffer and 400 units of T4 DNA ligase for 15 minutes at room temperature.

2.2.10.4 Transformation and screening: Three microliters of each Golden Gate and ligation reactions were transformed into Top10 F′, outgrown for 2.5 hours at 30° C., and two dilutions plated on bioassay trays.

Colonies were screened with the BamBgl.scr.F and TALE.scrR primers using Roche's 2×Kapa HiFi master mix. Glycerol stock cultures of the colonies were diluted in water: 10 μL of culture and 15 μL of water, and incubated at 95° C. for 5 minutes to lyse the cells. Four microliter PCR reactions were set up with 2 μL of the Kapa master mix, 0.25 μM each primer, and 0.2 μL of the lysed culture. Cycling conditions were 95° C. for 2 minutes; 10 cycles of 98° C. for 20 seconds, an annealing touchdown from 65° C. to 60° C. at 0.5° C. per cycle for 20 seconds, 72° C. for 2 minutes; followed by 20 cycles of 98° C. for 20 seconds, 60° C. for 20 seconds, 72° C. for 2 minutes; followed by a final extension of 72° C. for 3 minutes; and a hold at 4° C.

2.2.10.5 Golden Gate reactions transformed into DH10β(Δung): The primer-borne Golden Gate reactions were repeated using all of the enzymes in one-pot reactions, and transformed into DH10β(Δung) electrocompetent cells. Five fmoles of vector and 20 fmoles of insert was used in each 10 μL reaction, with 400 units of T4 DNA Ligase and 0.5 μL of enzyme, or 0.7 μL of the BsaI-HFv2 and BsmBI-v2 Golden Gate Enzyme mixes. Cycling was 30 cycles of 37° C. (42° C. for BsmBI) for 5 minutes and 16° C. for 5 minutes, followed by 16° C. for 1 hour, 65° C. for 10 minutes, and a hold at 4° C. In the morning an additional 120 units of ligase was added to each reaction, they were incubated for 1 hour at 25° C., 10 minutes at 65° C., and then held on ice for electroporation into DH10β(Δung). Colonies were PCR screened as above.

2.2.10.6 Direct screening of assembly reactions: One microliter of each assembly reaction was used as template for PCR screening with Kapa HiFi as above. PCR screening was repeated using Q5U polymerase for the dUTP assemblies. Ten microliter reactions with 1M betaine, 5 pmoles each of the primers, and 1 μL of assembly. Cycling was 98° C. for 30 seconds, 25 cycles of 98° C. for 10 seconds and 72° C. for 4 minutes; followed by a final extension of 72° C. for 2 minutes and a hold at 4° C.

2.2.11 Sequencing of PaqCI restriction digestion of 5mCTP amplified DNA. A part was amplified for Golden Gate assembly for another project. PaqCI-tailed primers were chosen to amplify this part using 5mCTP PCR. The amplicon was digested overnight with PaqCI. An aliquot of the digest was sequenced on a Pacific Biosciences Sequel IIe.

### 2.3 Results

The restriction enzymes tested were chosen as they were available in the lab. For the restriction digest tests some isoschizomers, enzymes which have the same recognition sequence and cleavage overhangs, were tested as isoschizomers can sometimes show different effects from DNA modifications, allowing the use of one in conditions in which the other will not work.

2.3.1 Effects of DNA modification on restriction digestion. FIGS. 5-7 show the effects of DNA modification on the restriction digestion of internal restriction sites. Either digestion appears complete, digestion appears incomplete (inhibited), or no apparent digestion is observed (blocked).

The general effects of DNA modification on digestion by various Type IIS and a handful of type IIP restriction enzymes is summarized in Table 2.4.

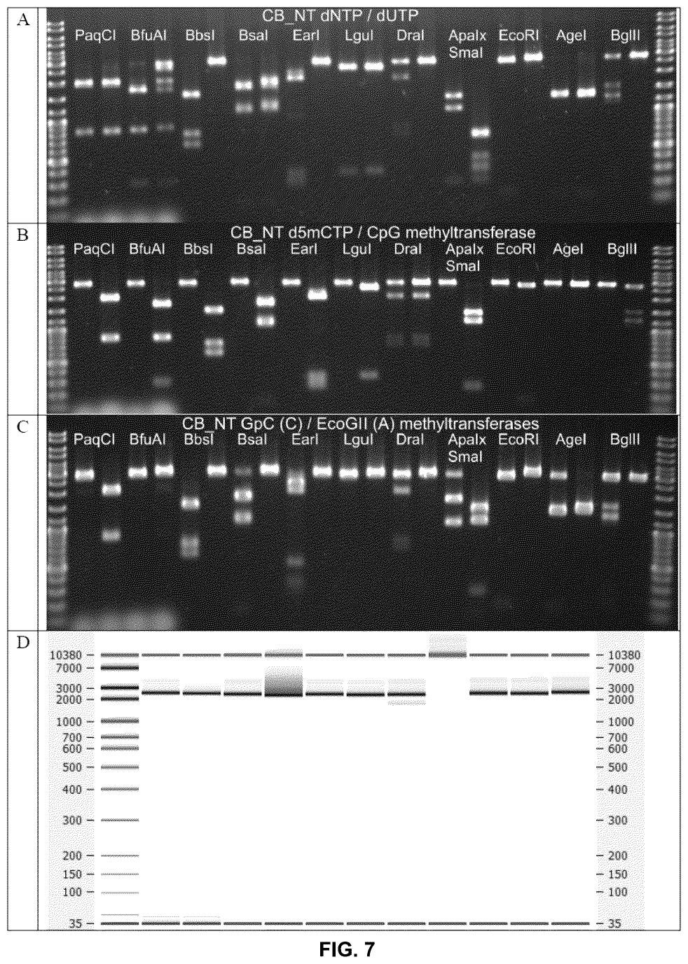

Some particular notes: LguI is greatly inhibited by GpC methylation (FIG. 5, panels B, C, D), but digestion increases as incubation time increases (FIG. 5, panel A). SapI is inhibited by overlapping CpG methylation (FIG. 5, panel B). EarI is blocked by GpC methylation of SapI/LguI sites (FIG. 5, panel B, D). BsmBI-v2 is greatly inhibited by both CpG and EcoGII methylation (FIG. 5, panel A, B, C), but may show some site-specific effects when digesting EcoGII methylated DNA (FIG. 5, panel B). At least one of the type IIP enzymes, ApaI or SmaI, shows star activity when uracil is substituted for thymine (FIG. 7, panel A).

Most intriguingly, BsaI-HFv2 shows partial inhibition of digestion at all sites in GpC methylated DNA, which should not happen as BsaI sites contain no GC sequences, and cannot overlap with GC sequences. Relative lane quantification analysis was performed using Bio-rad's Image Lab 6.1 software. For pR6k-2L-SmaI-Y, the ˜300 ng 15 hour digest of DNA from the 2 μg methylation showed an undigested band quantified as 17.1% of the total lane band intensity, while in FIG. 5, panel B, a 4 hour BsaI-HF digest of DNA from the 6 μg methylation showed the undigested band at 3.4% relative intensity. For the CB_NT amplicon, a 50 ng, 1 hour BsaI-HF digest DNA from a 1.7 μg methylation reaction (FIG. 7, panel C) showed undigested intensity of 13.7% total band intensity. The ApaIxSmaI and AgeI-HF digests showed 23.2% and 37.9% of total band intensity, respectively. For 346-6-GG-mod, a 4 hour digest of 300 ng of DNA from a 5-6 μg methylation showed undigested or partial digested intensity of under 5% for both bands. The more ‘highly methylated’ DNAs show a greater degree of BsaI-HFv2 inhibition. This inhibition is also shown by the SmaI digested DNA and the AgeI-HF digested DNA in the GpC methylated digests (FIG. 7, panel C). The only sequence feature that these three enzyme sites share in common are CC/GG dinucleotides.

The AgeI-HF digestion of EcoGII methylated DNA (FIG. 7, panel C) showed site-specific inhibition of digestion, lacking the ˜200 bp lower band visible in the GpC partial digest. Sequencing (not shown) showed that the AgeI site ctgcaACCGGTgaatg (SEQ ID NO:50) showed complete digestion, while the site aagagACCGGTctaac (SEQ ID NO:51) was greater than 85% blocked.

For the isoschizomers, AarI and PaqCI (CACCTGC(4/8)) acted basically the same on methylated DNA, and were only blocked by GpC methylation, while BfuAI (ACCTGC(4/8) was greatly inhibited by EcoGII methylation in addition to being blocked by GpC methylation. SapI (GCTCTTC(1/4)) was effectively blocked by GpC methylation while LguI was just greatly inhibited by it, and EarI (CTCTTC(1/4)) was inhibited at GpC methylated SapI/LguI sites. LguI and EarI also showed no effective inhibition by overlapping CpG methylation at SapI/LguI sites, while SapI was inhibited at those sites. Uracil substitution of thymine blocked EarI digestion, inhibited SapI, and had no apparent effect on LguI. 4mCTP inhibited LguI and blocked both EarI and SapI (FIG. 6). BbsI-HF (GAAGAC(2/6)) appeared more inhibited by EcoGII methylation than BpiI, but both were strongly inhibited. BsmBI-v2 (CGTCTC(1/5)) appeared slightly less inhibited on the gel than Esp3I at CpG and EcoGII methylated sites, with some site-specific effects on EcoGII methylated DNA (FIG. 5, panel B).

TABLE 2.3
Summary of digest results for FIGS. 5-7.
The Type IIS and HIP restriction enzymes, their recognition
sequences, the nucleotide content of their top strand recognition
sequence, and the effect of DNA modification on their digestion at
fully-modified recognition sites. [symbol] = no effect; [symbol] = digestion
inhibited; [symbol] = site-specific inhibition or blocking; [symbol] =
digestion blocked; [symbol] = at least one enzyme shows star activity; [symbol]
= gel shift; [symbol] = not tested
The Type IIS and IIP restriction enzymes, their recognition
sequences, the nucleotide content of their top strand recognition
sequence, and the effect of DNA modification on their digestion at
fully-modified recognition sites. [symbol] = no effect; [symbol] = digestion
inhibited; [symbol] = site-specific inhibition or blocking; [symbol] =
digestion blocked; [symbol] = at least one enzyme shows star activity; [symbol]
= gel shift; [symbol] = not tested.

| | | | | | | dUT | 7dGT | 4mCT | 5mCT | Cp | Gp | EcoG |
|---|---|---|---|---|---|---|---|---|---|---|---|---|
| | | A | T | C | G | P | P | P | P | G | C | II (A) |
| Type IIS | | | | | | | | | | | | |
| PaqCI | CACCTG | 1 | 1 | 4 | 1 | NE | B | NA | B | NE | B | NE |
| | C | | | | | | | | | | | |
| AarI | CACCTG | 1 | 1 | 4 | 1 | NA | NA | NA | NA | NE | B | NE |
| | C | | | | | | | | | | | |
| BfuAI | ACCTGC | 1 | 1 | 3 | 1 | I | B | B | B | NE | B | I |
| BbsI-HF | GAAGAC | 3 | | 1 | 2 | B | B | NA | B | NE | NE | B |
| BpiI | GAAGAC | 3 | | 1 | 2 | NA | NA | NA | NA | NE | NE | B |
| BsaI- | GGTCTC | | 2 | 2 | 2 | NE | B | NA | B | S | I | B |
| HFv2 | | | | | | | | | | | | |
| EarI | CTCTTC | | 3 | 3 | | B | B | B | B | NE | S | B |
| LguI | GCTCTT | | 3 | 3 | 1 | NE | B | I | B | NE | I | B |
| | C | | | | | | | | | | | |
| SapI | GCTCTT | | 3 | 3 | 1 | I | NA | B | NA | S | B | B |
| | C | | | | | | | | | | | |
| Esp3I | CGTCTC | | 2 | 3 | 1 | B | B | B | NA | I | NE | B |
| BsmBI- | CGTCTC | | 2 | 3 | 1 | B | B | B | NA | I | NE | I |
| v2 | | | | | | | | | | | | |
| Type IIP | | | | | | | | | | | | |
| DraI | TTTAAA | 3 | 3 | | | B | NE | NA | NE | NE | NE | B |
| ApaIx- | GGGCCC | | | 3 | 3 | — | ? | NA | B | NE/B | B/I | NE |
| Smal | CCCGGG | | | | | | | | | | | |
| EcoRI- | GAATTC | 2 | 2 | 1 | 1 | B | B | NA | B | NE | NE | B |
| HF | | | | | | | | | | | | |
| AgeI-HF | ACCGGT | 1 | 1 | 2 | 2 | NE | B | NA | B | B | I | S |
| BglII | AGATCT | 2 | 2 | 1 | 1 | B | B | NA | B | NE | NE | B |

The Type IIS and IIP restriction enzymes, their recognition sequences, the nucleotide content of their top strand recognition sequence, and the effect of DNA modification on their digestion at fully-modified recognition sites.

2.3.2 Effects of DNA modification on cloning. I performed transformations of modified and unmodified DNA, either on the vector only or after DNA assemblies (Gibson/In-Fusion and Golden Gate) to determine the effects that the DNA carrying the modified nucleotide analogs had on transformation efficiency and colony counts.

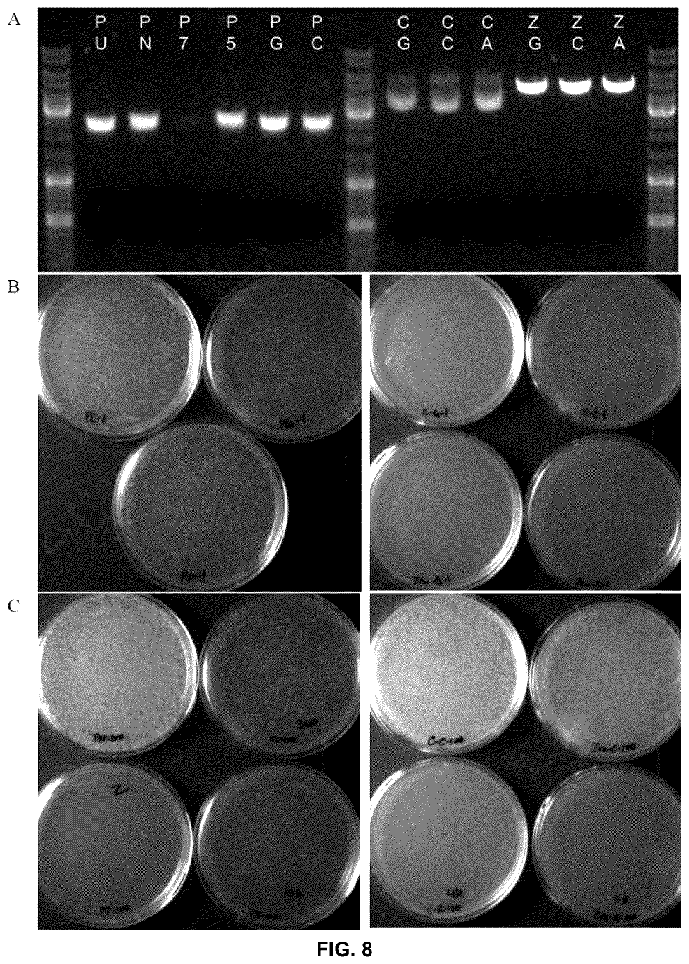

As seen in Table 2.4 and FIG. 8, methylation of a vector backbone (containing the origin of replication and antibiotic resistance gene) with CpG or GpC methyltransferases had no apparent effect on colony counts. However, hypermethylation of all cytosines or adenines resulted in about a 2-log decrease in colonies, with at least the 5mC backbone rescuing to a 1-log decrease with extended outgrowth for 3 hours at the lower temperature of 30° C. Transformation of undigested EcoGII methylated vector showed that the decrease in colonies, for EcoGII-treated DNA at least, is not due to a failure of the kinase or ligase to work on modified DNA (FIG. 8, panel C). Uracil-substituted vector backbone yielded no colonies in regular Top10 cells following a typical outgrowth period, but this was apparently rescued to approximately the same colony count as the 5mC backbone when also grown for 3 hours at 30° C. before plating on selective media (Table 2.4 A,B). Transformation of the uracil-substituted backbone into DH10β(Δung) cells resulted in about 3-fold more colonies than the 5mC backbone, but still much less than unmodified backbone (FIG. 8, panel C). 7dG substituted vector DNA showed a maximum of 2 colonies in one transformation (FIG. 8, panel C), almost 2-log lower than the 5mC vector. Typical PCR reactions for these experiments had 1 ng of linear template, and yielded on the order of 4 μg, plus or minus. The approximate 4-log decrease in 7dG substituted vector colonies was on the order of what would be expected from recircularization of the unmodified template DNA.

TABLE 2.4
Cloning of DNA modified in the vector backbone shows that
hypermethylation of C or A nucleotides inhibit colony counts.

| | A | B | C |
|---|---|---|---|
| Outgrowth: | 1 hour at 37° C. | 3 hours at 30° C. | 1 hour at 37° C. |
| pENTR-dNTP | Lots | Lots | ~1000 |
| pENTR-dUTP | 0 | ~10% of dNTP | NA |
| pENTR-5mCTP | Handful | ~10% of dNTP | NA |
| pENTR-7dGTP | 0 | 0 | NA |
| pENTR-CpG | NA | NA | ~1000 |
| pENTR-GpC | NA | NA | ~1000 |
| pENTR-EcoGII | NA | NA | 11 |

Colony counts from pENTR-SD-SmaI transformation plates. A) & B) 20 ng of linear DNA recircled by ligation, C) 50 ng of linear DNA recircled by ligation.

Colony counts from pENTR-SD-SmaI transformation plates. A) & B) 20 ng of linear DNA recircled by ligation, C) 50 ng of linear DNA recircled by ligation.

Chewback cloning of modified insert into non-modified vector (Table 2.5) again shows no inhibitory effect of CpG or GpC methylated DNA. Hypermethylation of all cytosines in an insert shows a slight inhibitory effect on colony counts, and EcoGII methylation of an insert shows a decrease in colonies by up to 1-log.

TABLE 2.5
Chewback cloning of modified DNA into an unmodified vector
shows that 5mC substitution had little effect while EcoGII methylation
had 1-log fewer colonies. Bioanalyzer results (not shown) demonstrated
that the polymerase and/or exonuclease in NEBuilder HiFi can't assemble
dUTP amplified DNA. Negative control reactions consist of only linear
vector or only insert. Outgrowths were for 1 hour at 37° C.

| | | NEBuilder | | InFusion |
|---|---|---|---|---|
| | | negative | | negative |
| | NEBuilder | controls | InFusion | controls |
| Vector only | NA | 1 | NA | 0 |
| dNTP insert | >100 | 4 | >>100 | 1 |
| dUTP insert | 0 | 0 | 3 | 0 |
| 7dGTP insert | 0 | 0 | 0 | 0 |
| 5mCTP insert | ~100 | 0 | NA | NA |
| CpG insert | >100 | 2 | NA | NA |
| GpC insert | >100 | 0 | NA | NA |
| EcoGII insert | 14 | 0 | ~50-80 | 0 |

Colony counts of the transformation plates. > = greater than; >> = much greater than; ~ = approximately; NA = not applicable.

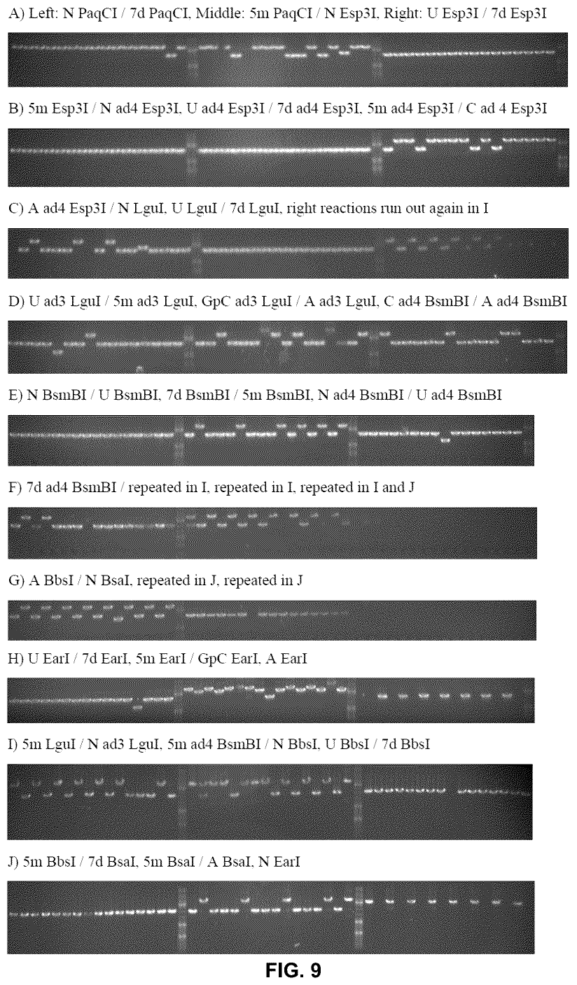

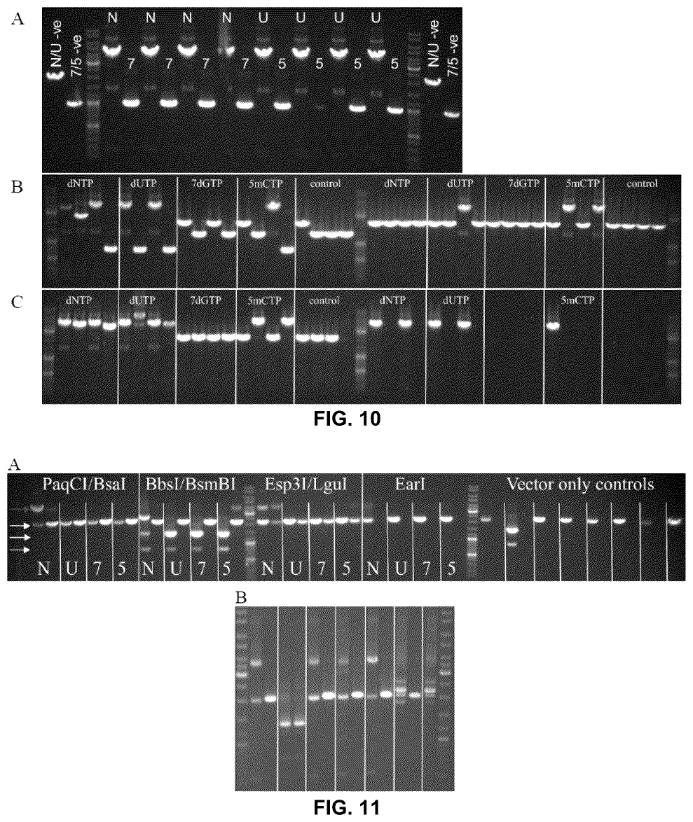

The Golden Gate and ligation cloning results are shown in FIG. 9 (summarized in Table 2.6) and FIG. 10. The initial set of assemblies were transformed into Top10 cells, which did not propagate the uracil-substituted assemblies. A second set of assemblies using only primer-borne sites and one-pot Golden Gate assembly was transformed into DH10β(Δung) (Example 3).

Colony counts and combinations of enzymes and insert modifications are in Appendix D for the FIG. 9 transformations into Top10 cells. Screening gels for both assembly sets showed that when inserts are amplified with unmodified flanking primers that PaqCI can digest and clone DNA amplified with 5mCTP, dUTP, and apparently even 7dGTP, though with many fewer colonies. BbsI-TIF does not effectively clone modified DNA when amplified with primer-borne sites. BsaI-HIFv2 can surprisingly clone EcoGII modified DNA from primer-borne sites, and can also clone uracil-substituted DNA, which is not surprising as uracil substitution does not block internal BsaI sites. Esp3J can clone CpG methylated, EcoGII methylated, or 5mCTP hypermethylated DNA when Esp3J sites are ligated to the DNA as fully unmodified adapters, and can effectively clone uracil substituted DNA from primer-borne sites. The CpG and 5mCTP reactions had a greater proportion of correct clones than the unmodified control, which is not surprising given the three internal Esp3I sites in the insert, while the EcoGII modified DNA had only 1 correct colony. Cloning with Esp3I's isoschizomer, BsmBI-v2, showed slightly less efficiency for the adapter-based cloning, but did show correct clones for non-adapter 5mCTP amplified DNA, and none for the unmodified control. BsmBI-v2 also showed no ability to clone uracil-substituted DNA based on two negative insert colonies. LguI and EarI likewise showed very good results from the non-adapter 5mCTP amplified DNA and uracil-substituted DNA, and LguI showed some correct colonies for the adapter-ligated 5mCTP, GpC, and EcoGII DNA.

Colony screens are interlaced. Each section of 16 screened clones is from two reactions, ranging from clones #1 in the left two lanes to clones #8 in the right two lanes. The 8 PaqCI 7dGTP PCR reactions were submitted for sequencing.

TABLE 2.6
Summary of the Top10 R6k-to-346 PCR screening gels.

| | PaqCI | BbsI | BsaI | Esp3I | BsmBI | LguI | EarI |
|---|---|---|---|---|---|---|---|
| N | 7/8 | 7.5/8 | 8/8 | 4/8 | 0/8 | 2/8 | 0*/8 |
| U | | 0/8 | 0/8 | 0/8 | 0/8 | 0/8 | 0/8 |
| 7 d | 8/8 | 0/8 | | 0/8 | 0/8 | 0/8 | 0/8 |
| 5 m | 7/8 | 0/8 | 0/8 | 0/8 | 6/8 | 7/8 | 7*/8 |
| GpC | | | | | | | 1*/8 |
| A | | 0/8 | 5/8 | | | | 0/8 |
| N adpt | | | | 0/8 | 0/8 | 0/8 | |
| U adpt | | | | 0/8 | 0/8 | 0/8 | |
| 7 d adpt | | | | 0/8 | 0/8 | | |
| 5 m adpt | | | | 5/8 | 3/8 | 1/8 | |
| CpG adpt | | | | 7/8 | 3/8 | | |
| GpC adpt | | | | | | 3*/8 | |
| A adpt | | | | 1/8 | 1/8 | 3/8 | |

Colony counts for the Golden Gate and ligation assemblies.
Red indicates no correct colonies, green indicates at least one correct colony.
The BbsI dNTP ligation had a mixed colony with correct and vector only bands.
The LguI GpC Adpt3 Golden Gate had two colonies with what appears to be two inserts.
The EarI dNTP ligation colonies appeared to have truncated inserts, possibly from mis-ligation on an internal EarI site.
This was also the case for the EarI GpC colonies, except for one colony that may have had been correct (clone #3).
Seven out of eight EarI 5mCTP colonies appear correct, including one colony with possible multiple inserts.

Direct PCR screens (FIG. 11) of the second set of Golden Gate assemblies transformed into DH10β(Δung) were performed to determine whether assemblies were successful even if no correct colonies were picked. The screens showed intense correctly assembled bands for PaqCI with unmodified and dUTP assemblies, and maybe a very faint band for the 5mCTP assembly. BsaI-HFv2 shows correct bands for unmodified and dUTP modified assemblies. The BbsI-HF results show a correct sized band for unmodified DNA, but the general misassembly of the backbone makes the BbsI-HF results difficult to interpret. BsmBI shows good assembly bands for 5mCTP and dUTP, and a fainter one for unmodified DNA. Esp3I shows good bands for dUTP and unmodified DNA. Both LguI and EarI show good bands for unmodified DNA and 5mCTP assemblies, and something weird for the dUTP assembly. These results and the result in FIG. 10 are summarized in Table 2.7.

2.3.3 Sequence fidelity of modified DNA assemblies, and digestion of modified DNA. PCR screens of 8 colonies of the PaqCI 7dGTP assembly, and 4 colonies each of the PaqCI dNTP and dUTP assemblies were sent for sequencing. The sequencing showed no mutations in these sequenced colonies.

The sequencing of 16 clones from the DH10β(Δung) Golden Gate transformations showed 14 sequence perfect clones. A BsmBI 5mCTP clone showed a C>T conversion in about half of reads, and an LguI dNTP clone showed a C>A conversion in all reads

TABLE 2.7
Summary of the R6k-to-346 PCR screening gels.

| | PaqCI | | BsaI-HFv2 | | BsmBI-v2 | | Esp3I | | EarI | | LguI | |
|---|---|---|---|---|---|---|---|---|---|---|---|---|
| | Plate | PCR | Plate | PCR | Plate | PCR | Plate | PCR | Plate | PCR | Plate | PCR |
| dNTP | + | + | − | + | − | F | + | + | + | + | + | + |
| dUTP | + | + | + | + | − | + | + | + | + | F | + | F |
| 7dGTP | − | − | − | − | − | − | − | − | − | − | − | − |
| 5mCTP | + | − | − | − | + | + | − | − | + | F | + | + |

This Table summarizes the results of FIGS. 10 and 11 and shows whether or not Golden Gate cloning from primer-borne sites resulted in correct colonies and/or correct assemblies.
For each enzyme the left column indicates correct colonies from the two-colony screening, and the right column indicates a full assembly band on the direct assembly screening PCR.
BbsI-HF results are not shown due to the misassembly from internal BbsI sites.

### 2.4 Discussion

Modified DNA can be used to facilitate Golden Gate cloning by blocking internal restriction enzyme sites while leaving primer-borne enzyme sites cleavable, or by ligating unmodified adapters to modified DNA. Typically, the less modified the DNA, the better, though inserts can generally tolerate modification more than vector backbone elements.

Sometimes it might be necessary to clone into a vector that contains internal enzyme sites that will be used to assemble the other pieces. Historically this would be done with restriction enzymes by separately digesting all pieces, purifying them, and then ligating them together. It may be possible to modify the vector backbone to silence those internal sites, thus allowing one-pot assembly. PCR of a vector with dUTP or 5mCTP seem to greatly reduce colony count, even when transformed into a Δung strain, while 7dGTP apparently eliminates colonies altogether. There may be an inhibitory effect of the modified nucleotides on T4 polynucleotide kinase, or T4 DNA ligase. Further studies are necessary to see whether this is the case. If a probable decrease in colonies is acceptable though, dUTP amplification of a vector could be used for one-pot, one-enzyme Golden Gate with EarI and Esp3I, possibly BsmBI-v2, and maybe with SapI, BfuAI, or other enzymes that are inhibited by uracil substitution. For the enzymes tested here, 5mCTP amplification could be used with PaqCI, BsmBI-v2, and LguI primer-borne sites.

For vectors, targeted methylation seems a better candidate, as the CpG and GpC methyltransferases show no inhibition of colonies. It would, however, be necessary to linearize the vector prior to methylation. This requires digestion with an enzyme that does not cut elsewhere within the vector, but the enzyme does not have to be the same one used for the fragments. This is basically the strategy I took with the R6k-to-346 Golden Gate and ligation reactions. While EcoGII could be used following linearization to block or inhibit all of the Type IIS sites studied here except PaqCI and AarI, the decrease in colonies may be problematic, especially for less efficient many-part assemblies. GpC methylation blocks PaqCI/AarI, BfuAI, and SapI, and inhibits BsaI-HFv2 and LguI. CpG methylation greatly inhibits the isoschizomers Esp3I and BsmBI-v2.

For the cloning of inserts, due to the nature of the DNA modifications tested here, BbsI is likely of use only with ligated adapters. Fortunately, adapter ligation demonstrates decent efficiencies for the cloning of single inserts. Surprisingly BsaI-HFv2 was able to clone EcoGII methylated DNA from primer-borne sites, when it is fully blocked at internal sites. This may indicate inefficient methylation close to the ends of a fragment, but such an explanation does not explain why BbsI-IF was apparently blocked by EcoGII methylation at primer-borne sites. Sequencing with protocols to detect methylation will be required to resolve this question.

While PCR modification with 100% nucleotide substitution can likely be counted on to fully modify all targeted bases, it will be important to ensure methylation by methyltransferases is complete enough to block internal sites. Alternatively, especially if using sequential restriction digest and ligation cloning, both PCR nucleotide substitution and methyltransferase methylation can be purposefully incomplete, with a restriction enzyme digestion and gel purification of the PCR products used to separate DNA fragments which have all internal sites blocked from those which do not. This is especially an approach to consider if modifying a vector backbone with 5mCTP PCR or EcoGII methylation, as incomplete methylation will theoretically result in more colonies. Whether partial substitution would also result in more colonies of partially substituted dUTP PCR is another question that I do not have a theoretical basis to guess at an answer. If partial uracil substitution is attempted, due to the demonstrated inefficiency of 100% dUTP PCR compared to 100% dTTP PCR with Q5U polymerase, it's unlikely that the ratios of dUTP to dTTP in a partial substitution reaction would directly equal the ratio of uracil to thymine incorporated into amplicons. Empirical testing would be required, and may differ based on the particularities of each amplicon.

Sequence analysis of the 5mCTP amplicon with primer-borne PaqCI sites showed only partial digestion at these sites after overnight incubations with PaqCI and AarI. NEB indicates 50%-100% cleavage with two additional base pairs off the end of a restriction site for PaqCI, and most of the other Type IIS enzymes tested here. But PCR does not always completely fill in the ends of amplicons, and errors within primers can inhibit digestion. I did not notice many possible primer errors in the sequencing, but addition of more nucleotides at the 5′ end of the primer may facilitate full cleavage, especially as I added only two thymines 5′ of the enzyme recognition sites, which may be more likely to breathe than C/G base pairs.

For whichever enzyme is chosen, and whether using a one-pot Golden Gate approach or two-pot restriction/ligation approach, a specific modification strategy will be required.

The ApaI and SmaI double digest of uracil-substituted DNA showed a star effect. While this was not seen with the other enzymes tested, it is possible star activity could happen with any other enzyme with the correct sequence context. Testing this possibility, especially with the 7-base enzymes, would be a large task. But it is something to keep in mind should an unexpected result occur in future assemblies. While star activity was only seen here in the uracil substituted DNA, nucleotide hypermethylation would not typically be seen in nature, and so may not be reported, and prior experiments using fully or partially substituted DNA test only a subset of all restriction enzymes.

The AgeI-IF digestion of EcoGII methylated DNA unexpectedly showed site-specific inhibition that was not seen in the GpC, dUTP, or unmodified digests. A site-specific inhibition, above and beyond the total inhibition, also seemed apparent in the BsmBI-v2 digest of EcoGII methylated linear pR6k-2L-SmaI-Y DNA. REBASE predicts 100% cleavage of AgeI sites even when both adenosines are methylated. NEB also reports that EcoGII will not methylate all adenosines in a plasmid (personal communication). Possible hypotheses are that the sequence context around the inhibited AgeI site and BsmBI site(s) are prone to hydrophobic interactions, or interactions with the engineered AgeI-IF and BsmBI-v2 enzymes, when particular adenosines are methylated. Regardless of the reason, these results, in addition to the differential digestion of PaqCI sites at the ends of 5mCTP amplified DNA, show that sequence context may be especially important when digesting modified DNA.

DNA with uracil substituting for thymine seemed to require an Ung deficient cloning strain. While 3-hour outgrowth at a lower incubation temperature of 30° C. seemed to rescue dUTP amplified vector backbone cloned into an Ung+ strain, a 2.5 hour outgrowth at 30° C. yielded no correct picked colonies of Golden Gate reactions with a dUTP amplified insert and unmodified vector which were cloned into an Ung+ strain. I'm leery of accepting the first result without more stringent experiments.

7dG substitution had a more profound impact on colonies, with effectively zero above theoretical background when fully substituted in the backbone. Single insert cloning with PaqCI Golden Gate did show positive clones with 7dGTP amplified inserts. The colony counts were below the background seen in failed reactions with other enzymes, which may in part be because the vector was linearized with PaqCI (this would allow PaqCI to continually digest the vector, while any incompletely digested vector in reactions with other enzymes would recircle on an incomplete PaqCI digest). Given that 4 out of 5 guanosines in the PaqCI recognition site would have been substituted by PCR, and given that complete substitution of dG with 7dG appears to block PaqCI digestion, and given that all adapter-based cloning of 7dG amplicons failed, and given the absence of correct colonies in the second PaqCI Golden Gate reactions using 7dGTP amplified inserts and transformed into DH10β(Δung), I am also leery of accepting these results without more stringent experiments.

A surprising result was the apparent permissiveness of GpC methyltransferase on CC/GG dinucleotides. The methylation protocol I used was very stringent, using an excess of enzyme, up to the maximum concentration of SAM recommended, and the longest recommended incubation time. Even then methylation of 1-2 μg of DNA showed greater off-site protection by GpC than the reactions with 5-6 μg of DNA, when detected by blocking of restriction enzymes. It is likely this off-site effect would be far less noticeable if using less stringent reaction conditions or more DNA. Whether apparent CC/GG methylation by GpC methyltransferase could be increased enough so that all BsaI, AgeI, and SmaI sites would be blocked is an open question, as is the effect of such an increase on colony counts.

### Example 3

### Generation of an Ung-DH10β Strain

### 3.1 Introduction

Ung, the E. coli uracil-DNA glycosylase enzyme, removes uracil (deaminated cytosine) from DNA, leaving an abasic site (Hayakawa, et al, 1978). This uracil excision activity occurs immediately upon DNA introduction into E. coli, prior to DNA replication (Warner, et al, 1981). In order to clone with uracil containing DNA this enzyme has to be knocked out of the E. coli genome. Doing so will allow uracil containing DNA to be cloned, and just as importantly will replace the uracil bases with thymine bases during replication. Older E. coli strains exist with ung deleted (Peter Weigele, NEB, personal communication), but their genotypes are lacking for robust cloning purposes. In this chapter I use a CRISPR-Cas9 editing strategy to edit the ung gene out of the DH10β cloning strain.

### 3.2 Materials and Methods

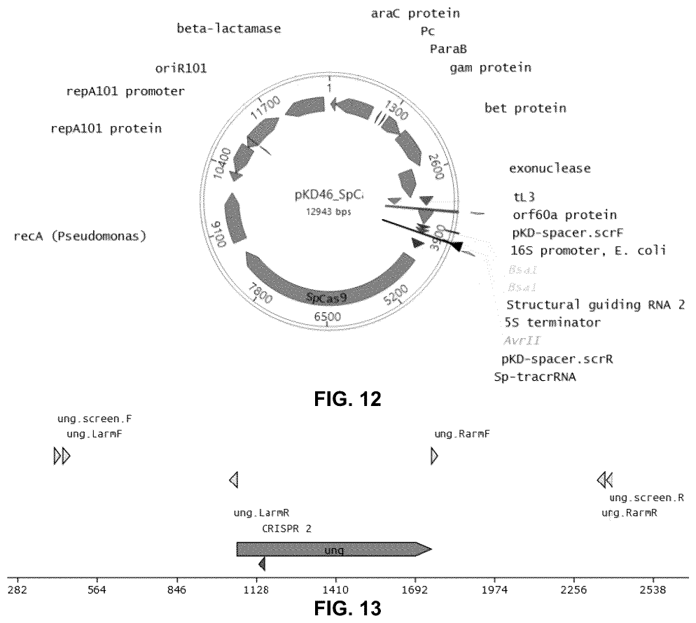

3.2.1 Editing plasmid. Plasmid pKD46-SpCas9-EcgRNA (FIG. 12) is used by the Joint Genome Institute's Synbio group for CRISPR-directed editing and recombinase-mediated, homologous repair in E. coli. This plasmid contains the Lambda red recombineering system consisting of the exo, gam, and bet genes driven by the arabinose-inducible ParaB promoter. It constitutively expresses the SpCas9 protein from the native Streptococcus pyogenes Cas9 promoter and sgRNA from the E. coli 16S promoter for double strand DNA cleavage at a targeted sequence. Its replication is driven by the heat-sensitive oriR101 origin of replication, allowing E. coli to be cured of the plasmid by incubation above 30° C. The homology arms are cloned into the AvrII site, and the 20 nt spacer is cloned into the BsaI sites.

3.2.2 Ung Gene sequence and oligos. The sequence and sequence context for ung (FIG. 13) was downloaded from the NCBI Nucleotide entry for the E. coli DH10B genome (Nucleotide).

Primers (Table 3.1) were designed from this sequence for PCR using NEB's Q5 polymerase, with melting temperatures using NEB's Tm Calculator (NEB, web). The primers amplify homology regions 620 bp upstream of the ung start site and 618 bp downstream of the ung stop codon, with 30 nt tails for NEBuilder assembly into the AvrII site of pKD46_SpCas9_EcgRNA, and 48 nt of overlap for PCR fusion to each other.

TABLE 3.1
List of oligos for Example 3.

| Primer name | Primer sequence |
|---|---|
| ung.LarmFF | ctaacttagttttccaactgttttccacgcctACTGCATAACCGGATACACGG |
| | ATGGT (SEQ ID NO: 52) |
| ung.LarmF | ACTGCATAACCGGATACACGGATGGT (SEQ ID NO: 53) |
| ung.LarmR | ATCTCGCTCTCCTGCGAATCTTCAATCC (SEQ ID NO: 54) |
| ung.LarmRR | aactctgccatccggcatttccccgcaaATCTCGCTCTCCTGCGAATCTTCAA |
| | TCC (SEQ ID NO: 55) |
| ung.RarmFF | gcggattgaagattcgcaggagagcgagATTTGCGGGGAAATGCCGGATG |
| | (SEQ ID NO: 56) |
| ung.RarmF | ATTTGCGGGGAAATGCCGGATG (SEQ ID NO: 57) |
| ung.RarmR | GGTGGATGAAGCGGAATTGACAAAAGC (SEQ ID NO: 58) |
| ung.RarmRR | ctccacaggatgatttcgtcagctgacgtcGGTGGATGAAGCGGAATTGACAA |
| | AAGC (SEQ ID NO: 59) |
| ung.screen.F | TGCTGTTCCGGAGTCAGAGAGT (SEQ ID NO: 60) |
| ung.screen.R | CAGCAAACCGCAAAGCGTACCA (SEQ ID NO: 61) |
| ung.spacerF | ttttGGACTGCCGCTCGCTGGCGA (SEQ ID NO: 62) |
| ung.spacerR | aaacTCGCCAGCGAGCGGCAGTCC (SEQ ID NO: 63) |
| pKD- | ACAGACGAAGAATCCATGACGTC (SEQ ID NO: 64) |
| spacer.scrF | |
| pKD- | GCGCTGCCATCTTGCTAAACCTAC (SEQ ID NO: 65) |
| spacer.scrR | |

3.2.3 PCR amplification and fusion of homology arms. DH10β cells were added to water and lysed at 95° C. for 5 minutes for use as a template. Oligo mixes were made of the Larm and Rarm oligos with the FF and RR oligos at 5 μM and the F and R oligos at 0.5 μM. PCR reactions were set up as follows:

| | 50 μL | L/Rarm PCR | | | | | Fusion PCR | |
|---|---|---|---|---|---|---|---|---|
| 5× buffer | 10 μL | 98° | C.-30 seconds | | | | 98° | C.-30 seconds |
| 5M betaine | 5 μL | 98° | C.-10 seconds | {close oversize brace} | 30 cycles | {open oversize brace} | 98° | C.-10 seconds |
| Water | 27.5 μL | 69° | C.-15 seconds | | | | 68° | C.-20 seconds |
| 10 mM dNTPs | 1 μL | 72° | C.-1 minute | | | | 72° | C.-4 minute |
| Q5 polymerase | 0.5 μL | 72° | C.-2 minutes | | | | 72° | C.-2 minutes |
| Oligo mix | 5 μL | 4° | C.-hold | | | | 4° | C.-hold |
| template | 1 μL | | | | | | | |

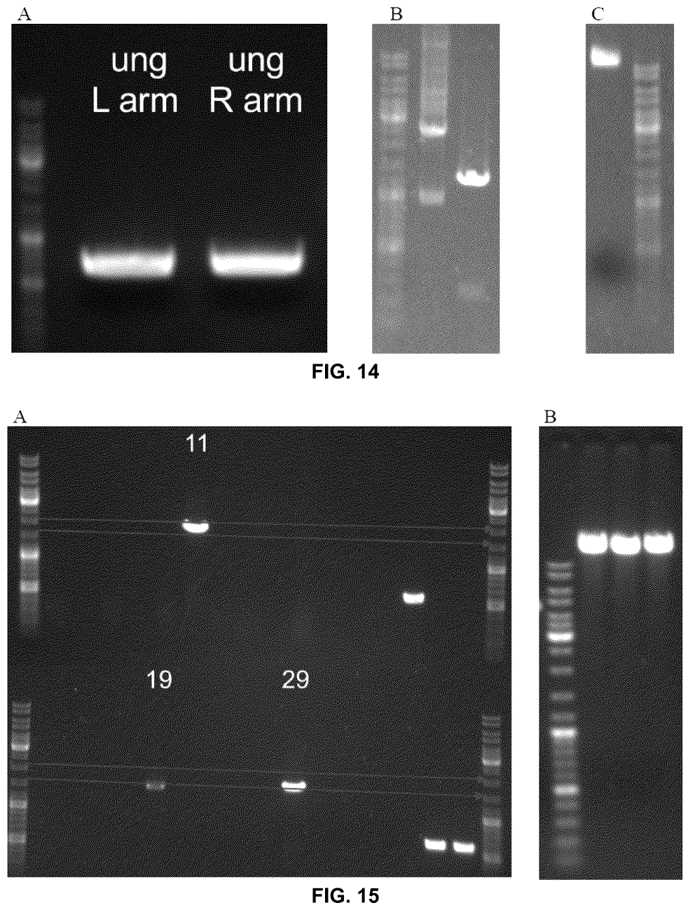

The PCR amplified homologous arms (FIG. 14, panel A) were gel purified using guanidine thiocyanate gel purification buffer and Zymo spin columns. They were then mixed and diluted for use as template for PCR fusion using a 5 μM oligo mix of the ung.LarmFF and ung.RarmRR oligos, and the PCR reaction above scaled to 20 μL. The fused arms (FIG. 14, panel B) were then gel purified.

3.2.4 Selection of guide RNA spacer sequence. Potential guide RNA spacers were generated using the Joint Genome Institute's gRNA-SeqRET tool (Simirenko, et al.; Table 3.2) with the ung coding sequence (CDS) as the target. Targets were sorted by CRISPRater score. The highest-scoring target, which is on the anti-sense strand near the 5′ end of the gene, was chosen. Complementary oligos were ordered with 4 nt tails for annealing and ligation into a BsaI digest of the pKD46-SpCas9-EcgRNA plasmid (ung.spacerF and ung.spacerR in Table 3.1).

3.2.5 Cloning of homology arms and spacer. Approximately 2.7 μg of pKD46-SpCas9-EcgRNA DNA was digested with 25 units of NEB's AvrII in a 50 μL reaction at 37° C. overnight (FIG. 14, panel C) and column purified. Thirty-seven nanograms of vector and 6.4 ng of fused homology arms were assembled for one hour at 50° C. in a 4 μL NEBuilder HiFi reaction and then transformed into chemically competent NEB Stable cells and plated on LB/Carb100 (100 mg/L) for overnight growth at 30° C. Thirty-two colonies were picked into LB/Carb100+7.5% glycerol medium and grown overnight at 30° C. Ten microliters of each glycerol stock was mixed with 15 μL of water and lysed at 95° C. for 5 minutes. This was used as template for PCR in 5 μL reactions with 0.3 μL of a 5 μM mix of the pKD-spacer.scrF and R screening primers, 0.5 μL template, and using Roche's 2×Kapa HiFi master mix. Cycling consisted of an initial denaturation for 2 minutes at 95° C., followed by 98° C. for 15 seconds in all cycles. The first 10 cycles were an annealing touchdown from 65° C. to 60° C. at 0.5° C. per cycle, with a 1 min extension step at 72° C., followed by an additional 20 cycles at 60° C. annealing temperature. A final extension step was 72° C. for 2 minutes, with a hold at 4° C.

Correct colonies gave an 1817 bp product and were bulked up in 100 mL of LB/Carb100+7.5% glycerol. They were midiprepped using Zymo Research's ZymoPURE midiprep kit and digested overnight with NEB's BsaI-HFv2. The following morning the digests were column purified. Approximately 1.9 μg of each DNA was treated with 1 μL of NEB's QuickCIP and an additional 1 μL of BsaI-HFv2 in a 20 μL reaction at 37° C. for 30 minutes with a 65° C. heat kill for 20 minutes.

The ung.spacerF and R oligos were resuspended at 100 μM using a buffer of 100 mM NaCl, 9.8 mM Tris-HCl, and 0.098 mM EDTA. They were then combined in equal amounts, raised to 95° C., and slowly cooled to 4° C. to anneal. Fifty picomoles (1 μL) was then phosphorylated with 10 units of NEB's T4 polynucleotide kinase in a 50 μL reaction using 1×T4 DNA Ligase buffer.

Ligation reactions were set up using 1 μL of the dephosphorylated vector reactions, 0.25 μL of the phosphorylated annealed spacers, 400 units of NEB's T4 DNA Ligase, and 1×T4 DNA ligase buffer in a 20 μL total volume. Ligations proceeded overnight on the benchtop.

One microliter each of the ligation reactions were electroporated into DH103 competent cells. They were outgrown for 2 hours at 30° C. with arabinose added to the medium at ≥0.02% and then plated on LB/Carb100 (with arabinose) for growth at 30° C. until pickable colonies were found. These colonies were picked into LB/Carb100+7.5% glycerol+arabinose cultures.

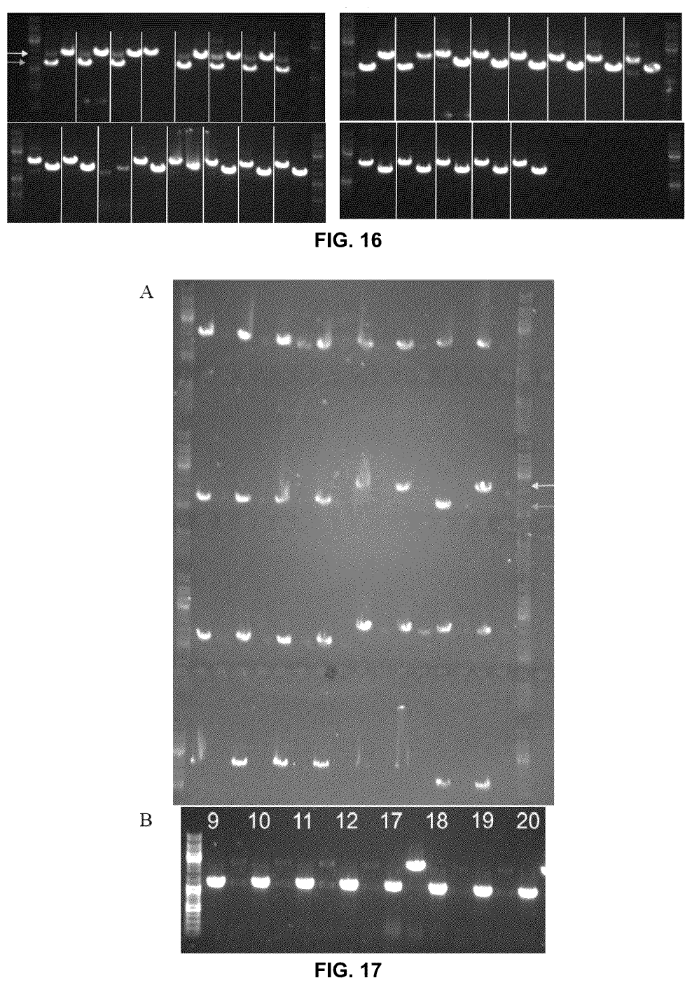

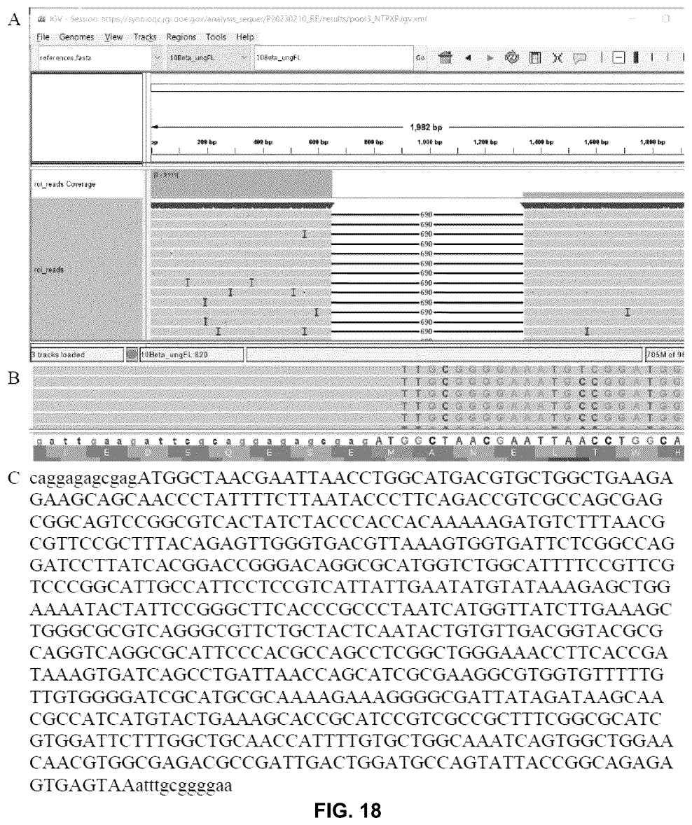

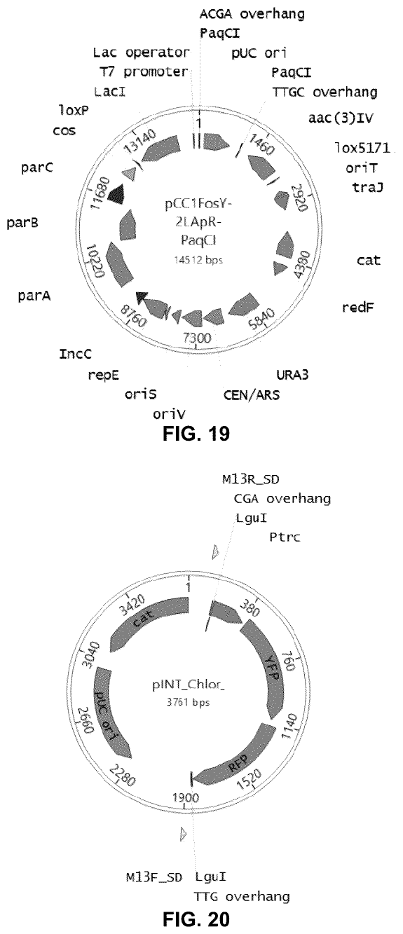

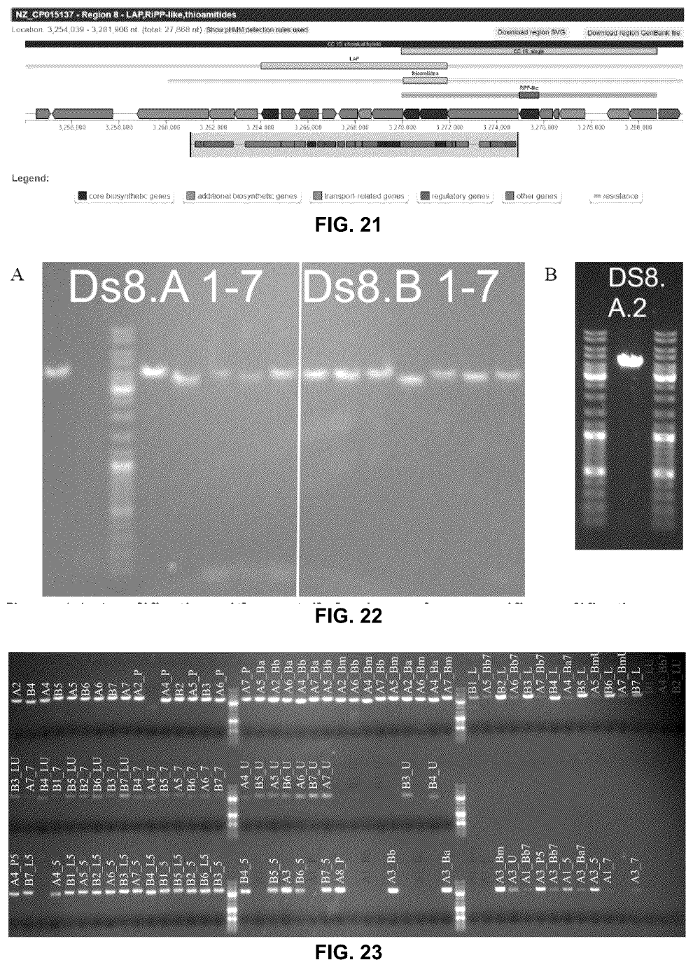

3.2.6 Generation of DH10β(Δung). The colonies were screened as above using the Kapa HiFi master mix, 35 total cycles, 90 second extensions, and using two reactions per colony. One reaction used the ung.LarmF and ung.RarmR oligos and the other used the pKD.screenF and R oligos (FIG. 16).

3.2.7 Preparation of competent cells. An online protocol for making electrocompetent DH10β at Rockefeller University's Laboratory of Molecular Parasitology was adapted for the equipment available in my lab (Rockefeller). Ten microliters from each of the 8 positive glycerol stocks were inoculated into a single 500 mL flask with 100 mL of SOB medium and grown overnight at 37° C. and 220 RPM. The next morning the OD600 was 5.33. Ninety milliliters of culture was split into two 4 L flasks containing 950 mL of SOB each and grown at 37° C. Initial OD 600 was 0.232 and 0.242; it took approximately 75 minutes for the OD600 to increase to 0.747 and 0.751. The cells were spun in six centrifuge bottles, with two being spun twice. The cells were initially resuspended in 1.5 L of ice-cold water. They were then resuspended in 500 mL of ice-cold water. There was no third water wash. They were finally washed in 50 mL of ice-cold 10% glycerol. At all wash steps care was taken to remove most residual medium, water, or 10% glycerol from bottles, tubes, and caps. Cells were resuspended with 4 mL of ice-cold 10% glycerol. Cells were aliquoted into breakaway plates and PCR strips tubes and frozen on dry ice before being placed at −80° C.

3.2.8 Testing of competent cells. One nanogram of a 14.5 kb plasmid was electroporated into a 40 μL aliquot of cells using a 2 mM gap cuvette, 2.4 kV, 200 Ω, 25 μF using an ECM 630 electroporation system from BTX. This was outgrown with 1 mL of SOC medium for 1 hour at 37° C., and plated on selective LB/agar.

### 3.3 Results

The ung gene was successfully knocked out of DH10β, generating DH10β(Δung). The ability to propagate uracil-containing plasmids is shown in chapters 2 and 4.

3.3.1 Construction of the CRISPR-Cas9 editing plasmid. CRISPR-Cas9 editing with the pKD46-SpCas9-EcgRNA plasmid requires sequential addition of a 20 bp spacer to guide double-strand cleavage of the target DNA, and a homologous repair template that lacks the spacer sequence, to the plasmid. Editing the genome of the cloning strain requires first cloning in the homologous repair template, and only then cloning in the spacer sequence, as constitutive CRISPR-Cas9 expression would otherwise cause immediate genome cleavage that would have to be repaired without a template.

In FIG. 14, panel A the DNA immediately flanking the ung gene are amplified with tails for PCR fusion together (FIG. 14, panel B) and Gibson assembly into the AvrII linearized plasmid (FIG. 3.3C). Thirty-two colonies were screened (FIG. 15), and the three with large inserts were bulked up and digested with BsaI-HFv2 for cloning of the spacer. gRNA-seqRET identified a total of 87 SpCas9 spacer sequences in ung, and the top scoring one (Table 3.2) was synthesized and ligated into the three digested vector clones.

Addition of arabinose was required at all step of the final cloning process, even during transformation outgrowth, to allow expression of the Lambda Red recombination genes to help repair the double-strand break from the constitutively expressed CRISPR-Cas9.

TABLE 3.2
The top scoring CRISPRater SpCas9 spacers
in the ung CDS. gRNA-SeqRET identified a
total of 87 SpCas9 spacers in the ung CDS.
T67, with protospacer in bold, was the top
scoring spacer.

| | | | |
|---|---|---|---|
| T67 | GGACTGCCGCTCGCTGGCGACGG | CRISPRater | 0.82963962 |
| | (SEQ ID NO: 66) | score | |
| T30 | TCGCCGCTTTCGGCGCATCGTGG | CRISPRater | 0.806179945 |
| | (SEQ ID NO: 67) | score | |
| T32 | TGGCTGCAACCATTTTGTGCTGG | CRISPRater | 0.777979245 |
| | (SEQ ID NO: 68) | score | |
| T33 | ATTTTGTGCTGGCAAATCAGTGG | CRISPRater | 0.77491307 |
| | (SEQ ID NO: 69) | score | |
| T68 | GCTCGCTGGCGACGGTCTGAAGG | CRISPRater | 0.76824208 |
| | (SEQ ID NO: 70) | score | |
| T78 | GAAGGTTTCCCAGCCGAGGCTGG | CRISPRater | 0.76198486 |
| | (SEQ ID NO: 71) | score | |
| T6 | AGAGTTGGGTGACGTTAAAGTGG | CRISPRater | 0.76077244 |
| | (SEQ ID NO: 72) | score | |
| T25 | AAGGCGTGGTGTTTTTGTTGTGG | CRISPRater | 0.75896824 |
| | (SEQ ID NO: 73) | score | |
| T21 | ACTGTGTTGACGGTACGCGCAGG | CRISPRater | 0.75271217 |
| | (SEQ ID NO: 74) | score | |
| T26 | GGCGTGGTGTTTTTGTTGTGGGG | CRISPRater | 0.75165267 |
| | (SEQ ID NO: 75) | score | |
| T9 | CTCGGCCAGGATCCTTATCACGG | CRISPRater | 0.749745395 |
| | (SEQ ID NO: 76) | score | |
| T12 | CACGGACCGGGACAGGCGCATGG | CRISPRater | 0.74817709 |
| | (SEQ ID NO: 77) | score | |
| T41 | AATCCACGATGCGCCGAAAGCGG | CRISPRater | 0.742973855 |
| | (SEQ ID NO: 78) | score | |
| T20 | CTTGAAAGCTGGGCGCGTCAGGG | CRISPRater | 0.74094566 |
| | (SEQ ID NO: 79) | score | |
| T7 | GACGTTAAAGTGGTGATTCTCGG | CRISPRater | 0.74024756 |
| | (SEQ ID NO: 80) | score | |
| T63 | GCGTTAAAGACATCTTTTTGTGG | CRISPRater | 0.73409369 |
| | (SEQ ID NO: 81) | score | |
| T45 | AATCAGGCTGATCACTTTATCGG | CRISPRater | 0.73146564 |
| | (SEQ ID NO: 82) | score | |
| T37 | TGACTGGATGCCAGTATTACCGG | CRISPRater | 0.73040729 |
| | (SEQ ID NO: 83) | score | |
| T56 | TGACGGAGGAATGGCAATGCCGG | CRISPRater | 0.73040729 |
| | (SEQ ID NO: 84) | score | |
| T46 | CGGTGAAGGTTTCCCAGCCGAGG | CRISPRater | 0.72941414 |
| | (SEQ ID NO: 85) | score | |
| T62 | CGTCACCCAACTCTGTAAAGCGG | CRISPRater | 0.72879647 |
| | (SEQ ID NO: 86) | score | |
| T52 | TGATTAGGGCGGGTGAAGCCCGG | CRISPRater | 0.728698005 |
| | (SEQ ID NO: 87) | score | |
| T74 | GACGCCGGACTGCCGCTCGCTGG | CRISPRater | 0.72713789 |
| | (SEQ ID NO: 88) | score | |
| T85 | TTCCCAGCCGAGGCTGGCGTGGG | CRISPRater | 0.72713789 |
| | (SEQ ID NO: 89) | score | |
| T61 | CCGGTCCGTGATAAGGATCCTGG | CRISPRater | 0.721390625 |
| | (SEQ ID NO: 90) | score | |
| T79 | AGGCGTGGTGTTTTTGTTGTGGG | CRISPRater | 0.721390625 |
| | (SEQ ID NO: 91) | score | |
| T38 | TCACTCTCTGCCGGTAATACTGG | CRISPRater | 0.721030375 |
| | (SEQ ID NO: 92) | score | |
| T83 | ATTCCCACGCCAGCCTCGGCTGG | CRISPRater | 0.72052042 |
| | (SEQ ID NO: 93) | score | |
| T47 | TTTCCCAGCCGAGGCTGGCGTGG | CRISPRater | 0.719308 |
| | (SEQ ID NO: 94) | score | |
| T35 | AATCAGTGGCTGGAACAACGTGG | CRISPRater | 0.717955475 |
| | (SEQ ID NO: 95) | score | |
| T17 | GAGCTGGAAAATACTATTCCGGG | CRISPRater | 0.716590155 |
| | (SEQ ID NO: 96) | score | |
| T16 | AGAGCTGGAAAATACTATTCCGG | CRISPRater | 0.71259089 |
| | (SEQ ID NO: 97) | score | |
| T64 | TTAAAGACATCTTTTTGTGGTGG | CRISPRater | 0.711134385 |
| | (SEQ ID NO: 98) | score | |
| T18 | GGCTTCACCCGCCCTAATCATGG | CRISPRater | 0.702281155 |
| | (SEQ ID NO: 99) | score | |

3.3.2 Screening of ung deletion colonies. A total of 10, 8, and 11 colonies for the spacer cloned into the #11, #19, and #29 vectors (FIG. 15) were present on the plates. After growth in culture at 30° C. these were PCR screened (FIG. 16) to select colonies for curation. The screening primers targeted the ung homology arms present in both the genome and vector which was expected to show a double band, with a larger genome band still possibly containing the ung gene fainter than the more abundant repair template in the low-copy vector. The second set of screening primers targeted only the vector to verify that it was present and intact.

3.3.3 Curing of the editing plasmid. Colonies that passed screening were struck out onto Spectinomycin (50 mg/mL) plates and grown overnight at 37° C. to prevent replication of the plasmid. Approximately four colonies from each were picked into LB/Spec50+7.5% glycerol cultures and again grown overnight at 37° C. Following this curation the picked colonies were screened again (FIG. 17, panel A). Eight colonies that showed the presence of the expected-sized ung locus, and absence of the pKD plasmid, were screened again using the genome-specific ung.screen.F and R primers (FIG. 17, panel B) and sent for sequencing on a Pacific Biosciences Sequel IIe (FIG. 7) to verify deletion of the ung gene.

3.3.4 Competent cell prep. The plate with 1/10,000^{th} of the transformation outgrowth, representing 0.1 pg of plasmid DNA, had 73 colonies. Multiplied by 10,000,000 pg/μg this yields 7.3×10⁸ Colony Forming Units (CFUs) per microgram of pCC1FosY-2LApR-PaqCI DNA.

### 3.4 Discussion

DH10β was chosen to mutate as it has a variety of properties beneficial to cloning, such as transformability of large plasmids, and lack of endonucleases.

Deletion of the ung gene allows cloning of DNA containing uracil nucleotides without immediately causing a DNA repair response (Warner, et al, 1981). This will allow ligation-based cloning, such as one-pot Golden Gate protocols, with PCR products that have uracil instead of thymine in order to silence internal restriction sites.

As shown previously (Duncan BK, Weis B, 1982), the deletion of ung results in a 20- to 30-fold increase of G:C>A:T mutations, as Ung removes deaminated cytosine bases, which are chemically identical to uracil, allowing the DNA repair machinery to replace the abasic site from the opposite strand template. Over time this will lead to a variety of mutations in the DH10β(Δung) strain, but more importantly can also cause mutations in cloned constructs, especially those which are GC-rich, or may be under counter-selective pressure in E. coli. As such this strain should only be used for DNA assembly, not for large-scale plasmid bulkup or propagation.

There are a variety of E. coli, and other bacteria, cloning strains with special properties such as stability of unstable constructs, and maintenance, reduction, or amplification of various origins of replication. It may be desirable to create Δung versions of some of these strains.

### Example 4

Golden Gate Cloning of a 28 kb Biosynthetic Gene Cluster from Dickeya Solani Using Nucleotide Modified DNA

This thesis project began as a comparison of techniques for the cloning of medium and large biosynthetic gene clusters (BGCs) directly from bacterial genomes. These are stretches of DNA from about 20 kb to over 100 kb which contain genes encoding a pathway for the synthesis of secondary metabolites. One of these techniques was to amplify out parts and assemble them with restriction enzyme digest and ligation. For the ligation of many parts non-palindromic overhangs are greatly preferred both for assembly efficiency, and to avoid unintended joining of parts. This limits the possibly enzymes to Type IIS enzymes and a few type IIP enzymes which cleave to leave degenerate overhangs.

For many BGCs, especially the longer ones, the Type IIS and IIP enzymes that can be used for this purpose are all present within the BGC. This makes one-pot Golden Gate assemblies problematic. Multi-pot assemblies are possible, though even then some BGCs will have regions of DNA in which almost every enzyme site is present. It is possible to use some of these internal restriction sites for assembly, but secondary structure around the sites can be problematic for the design of PCR primers. Regardless, the presence of internal sites makes restriction enzyme assembly of BGCs a tedious design process.

With this in mind I decided to revisit the MASTER ligation technique (Chen, et al., 2013) as mentioned in chapter 2. For reasons mentioned there I was leery about using it for the assembly of many parts. The methylation requirement of the MspJI restriction enzyme, along with notes of methylation sensitivity for the Type IIS enzymes on NEB's website, suddenly made me wonder whether post-hoc methylation of PCR products could be used to block the internal restriction sites. With a bit of research, detailed in the earlier chapters, this idea developed into the PCR and methyltransferase techniques detailed in chapter 2.

Originally, however, the main point is still the cloning of medium and large BGCs. In this chapter I amplify a 28 kb BGC in 7 parts, using a variety of Type IIS enzymes and all DNA modification strategies first tested in chapter 2, and assemble the BGC into a vector using both the primer-borne Type IIS site PCR-based approach and the ligated-adapter based approach. This demonstrates that both approaches work, as do most of the modifications, but as previously seen in chapter 2 not for all Type IIS enzyme and DNA modification combinations. See Appendix A for a list of abbreviations used in this chapter.

### 4.2 Materials and Methods

4.2.1 Cloning vectors. The primary cloning vector, pCC1FosY-2LApR-PaqCI, is 14.5 kb in length. Based on the copy control vector developed by EpiCentre it has a single-copy BAC origin of replication and an inducible origin of replication in the backbone. It has a CEN/ARS and ura3 yeast cassette for replication in S. cerevisiae. And it has a high-copy pUC origin of replication, for high-copy vector bulkup in E. coli, which pops out of the cloning site on a PaqCI digest. It has a partition locus to ensure plasmid inheritance. It has mobilization genes for conjugation into other bacteria. And it has an expression cassette consisting of a lacI and a lacO-controlled T7 promoter for inducible expression on the 5′ end of the cloning site and an Apramycin resistance gene for antibiotic selection on the 3′ end of the cloning site, which is flanked by loxP and lox5171 sites for Cre-mediated recombination into a host genome (Wang, et al., 2019). Other than the cloning site it is free of PaqCI (AarI) sites and BsaI sites, but contains multiple BbsI, BsmBI (Esp3I), LguI (SapI), and additional EarI sites. Digestion with PaqCI leave 5′ TGCT (ACGA) and 3′ TTGC overhangs.

The secondary cloning vectors, pINT-Chlor-FP-PaqCI and pINT-Chlor-FP-LguI have a pUC origin of replication and chloramphenicol (cat) resistance marker. Digestion with PaqCI or LguI removes a tandem RFP-YFP fluorescent protein cassette driven by a trc promoter and leaves 5′ TGCT (ACGA) and 3′ TTGC overhangs for the PaqCI digest, or 5′ GCT (CGA) and 3′ TTG overhangs for the LguI digest. The vector backbone lacks all Type IIS restriction sites used in this thesis.

Vectors were prepared for cloning by overnight digestion with an excess of PaqCI or LguI, dephosphorylation with NEB's QuickCIP, and gel purification. Some of the pCC1FosY-2LApR-PaqCI digest was methylated with CpG methyltransferase following the protocol in chapter 2, section 2.3.3.

4.2.2 Oligos for Example 4. Primers for PCR and adapter oligos were ordered from IDT-DNA with standard desalting. For adapters, restriction enzyme sites are in uppercase, the rest are in lowercase, and the T overhang is underlined. As in chapter 2, the adapter oligos were designed based on the MASTER adapter and validated with IDT-DNA's oligo analyzer tool (IDT-DNA, web). The adapters were ordered with 5′ phosphates and self-annealed.

For cloning oligos only the non-tailed primers are shown. With the exception of the first nucleotide of Ds8.A.01-F, all nucleotides are complementary to the template. Golden Gate overhangs are underlined. All cloning and screening primers were designed to minimize ΔTm of the primer pairs (Li, et al., 2011).

For the Type IIS tailed cloning oligos, the 1 to 4 nucleotide sequence between the enzyme recognition site and the overhang allowed addition of further template-complementary nucleotides, and consequent removal of nucleotides from the 3′ end of the oligo. A 72° C. annealing temperature was targeted for the complementary part of the oligos using NEB's Tm Calculator with the Q5 setting (NEB, web), except for primer sets for parts Ds8.A.01, which targeted a 67° C. annealing temperature, and Ds8.A.03, which targeted a 69° C. annealing temperature. For the restriction enzyme site tailed oligos the naming convention was to append the enzyme name (e.g. Ds8.A.01_PaqCI-F). The tails, appended to the 5′ end of the oligo, were: tttCACCTGCnnnn (PaqCI) (SEQ ID NO:100), ttGAAGACnn (BbsI), ttGGTCTCn (BsaI), ttCGTCTCn (BsmBI), and ttGCTCTTCn (LguI). Ns indicate additional template-specific nucleotides, and the enzyme sites are uppercase. PaqCI, BbsI, BsaI, and BsmBI tails were attached to the A set primers. LguI tails were attached to the B set primers.

Screening primers for the BGC cloned into the pCC1FosY-2LApR-PaqCI were designed using Primer-BLAST (Ye, et al., 2012). The forward screening primer, pCC1Fos-T7-LacO_scrF, was designed by hand to minimize secondary structure and entered into Primer-BLAST. The targeted primer Tm was adjusted to be within 1° C. of pCC1Fos-T7-LacO_scrF. The 41.4 kb sequence of the full-length BGC cloned into the vector was used as the PCR template. E. coli was set as the exclusion organism. The screening primers (Table 4.1) were designed to span the entire BGC, with overlap between the amplicons. A target amplicon length was set between 9 kb and 11 kb. 10-mer tail barcodes were attached to the 5′ ends of the screening primers for deconvolution in our sequencing pipeline. The tails are: AATTGGCACA (set1) (SEQ ID NO:101), ACCAGGAATT (set2) (SEQ ID NO:102), ATAGCCGGTT (set3) (SEQ ID NO:103), TGCTTCCAAG (set4) (SEQ ID NO:104), and CCGAAGGTTC (set5) (SEQ ID NO:105). For the pINT vector the vector-specific primers, M13R_SD and M13F_SD, were not barcoded.

TABLE 4.1
List of oligos for Example 4.
Adapter oligos

| | |
|---|---|
| PaqCI-TA | ggtGCAGGTGGatccctttttttagggatCCACCTGCacct |
| BtgZI-BbsI- | gGTCTTCaCATCGCggatcccttittttagggatccGCGATGtGAAGACct |
| TA | |
| BsaI-TA | GAGACCgatccctttttnagggatcGGTCTCt |
| Esp3I-TA | GAGACGgateccttttittagggatcCGTCTCt |
| LeuI-TA | GAAGAGCgatccctttttttagggatcGCTCTTCt |

Cloning oligos

| Ds8.A.01-F | aCGATCATGGCGTTCTTCATCC | | |
|---|---|---|---|
| Ds8.A.01-R | ACTCTAATTCTTGAGCCTTATCCATATGACT | | |
| Ds8.A.02-F | GAGTTGGCCGTTGCTGAACTGAATGC | | |
| Ds8.A.02-R | GATGCGAGGGTGATCCGATCGAAC | | |
| Ds8.A.03-F | CATCTCAGGTGACCATAGACACCA | | |
| Ds8.A.03-R | AATAACTACCCAACGCAACGCTTCTG | | |
| Ds8.A.04-F | TATTTTACCGCATCGGCTCTACTCACACT | | |
| Ds8.A.04-R | AAAGTGCGTTACCGTTTCAGGTTGCA | | |
| Ds8.A.05-F | CTTTTACCAGTCTTTCGGTCAGTTCACCG | | |
| Ds8.A.05-R | AGAATTGATTTCAGCGAGACGGTATTTTTCTOGG | | |
| Ds8.A.06-F | TTCTGACCGATCCGCCGTCACT | | |
| Ds8.A.06-R | CTCATCATAACTGAGTGGGGCCAGGG | | |
| Ds8.A.07-F | TGAGTACAACGTGCTGATTGAAAAAGGTTGTCT | | |
| Ds8.A.07-R | GCAACGGTTTACAAAGTCAGCGTCAACA | | |
| Ds8.B.01-F | CGATCATGGCGTTCTTCATCCTCTATTTCTGTCT | | |
| Ds8.B.01-R | TATTTTCTGCATTCAGTTCAGCAACGGCCA | | |
| Ds8.B.02-F | ATACTGAAGCTGAAAATGAAATTCTGCTGGATGTCAC | | |
| Ds8.B.02-R | GAGGGTGATCCGATCGAACAGCC | | |
| Ds8.B.03-F | CTCGCATCTCAGGTGACCATAGACACC | | |
| Ds8.B.03-R | AACGCTTCTGAAATGCCCCAGCT | | |
| Ds8.B.04-F | GTTGCGTTGGGTAGTTATTTTACCGCATCG | | |
| Ds8.B.04-R | TGCGTTACCGTTTCAGGTTGCAGG | | |
| Ds8.B.05-F | GCACTTTTACCAGTCTTTCGGTCAGTTCACC | | |
| Ds8.B.05-R | GGATCAATCGTCCAGACCTGCGC | | |
| Ds8.B.06-F | TCCCGAGAAAAATACCGTCTCGCTGA | | |
| Ds8.B.06-R | AGGGCTTTGCTGCTTTTTGTGAATCGG | | |
| Ds8.B.07-F | CCTGGCCCCACTCAGTTATGATGAGT | | |
| Ds8.B.07-R | CAACGGTTTACAAAGTCAGCGTCAACATCG | | |
| | | Amplicon | |
| | Screening oligos | length | |
| pCCIFos-T7- | GACTCACTATAGGGGAATTGTGAGCGGA | 10.9 | kb |
| LacO_serF | | | |
| Ds8_scrR1 | TCGGCCCCATACTGACGGGA | | |
| Ds8_scrF2 | TTGCGGGGGCAGTCTGGAAG | | |
| Ds8_scrR2 | CGACGACGCCTGTGGGGAAA | 10 | kb |
| Ds8_scrF3 | TACAATCGCAGTGGCOGGGAG | 9.9 | kb |
| pCC1Fos- | CAGCTCCGCGAAGTCGCTCT | | |
| pSET_scrR | | | |
| M13R_SD | AGAGCTGCCAGGAAACAGCTATGAC | 10.8 | kb |
| M13F_SD | ACGACGTTGTAAAACGACGGCCAG | 7.8 | kb |

Adapter oligos

| | |
|---|---|
| PaqCI-TA | ggtGCAGGTGGatccctttttttagggatCCACCTGCacct (SEQ ID |
| | NO: 106) |
| BtgZI-BbsI- | gGTCTTCaCATCGCggatccctttttttagggatccGCGATGtGAAGACct |
| TA | (SEQ ID NO: 107) |
| BsaI-TA | GAGACCgatccctttttttagggatcGGTCTCt (SEQ ID NO: 108) |
| Esp31-TA | GAGACGgatccctttttttagggatcCGTCTCt (SEQ ID NO: 109) |
| Lgul-TA | GAAGAGCgatccctttttttagggatcGCTCTTCt (SEQ ID NO: 110) |

Cloning oligos

| Ds8.A.01-F | aCGATCATGGCGTTCTTCATCC (SEQ ID NO: 111) | | |
|---|---|---|---|
| Ds8.A.01-R | ACTCTAATTCTTGAGCCTTATCCATATGACT (SEQ ID NO: 112) | | |
| Ds8.A.02-F | GAGTTGGCCGTTGCTGAACTGAATGC (SEQ ID NO: 113) | | |
| Ds8.A.02-R | GATGCGAGGGTGATCCGATCGAAC (SEQ ID NO: 114) | | |
| Ds8.A.03-F | CATCTCAGGTGACCATAGACACCA (SEQ ID NO: 115) | | |
| Ds8.A.03-R | AATAACTACCCAACGCAACGCTTCTG (SEQ ID NO: 116) | | |
| Ds8.A.04-F | TATTTTACCGCATCGGCTCTACTCACACT (SEQ ID NO: 117) | | |
| Ds8.A.04-R | AAAGTGCGTTACCGTTTCAGGTTGCA (SEQ ID NO: 118) | | |
| Ds8.A.05-F | CTTTTACCAGTCTTTCGGTCAGTTCACCG (SEQ ID NO: 119) | | |
| Ds8.A.05-R | AGAATTGATTTCAGCGAGACGGTATTTTTCTCGG (SEQ ID NO: 120) | | |
| Ds8.A.06-F | TTCTGACCGATCCGCCGTCACT (SEQ ID NO: 121) | | |
| Ds8.A.06-R | CTCATCATAACTGAGTGGGGCCAGGG [SEQ ID NO: 122) | | |
| Ds8.A.07-F | TGAGTACAACGTGCTGATTGAAAAAGGTTGTCT [SEQ ID NO: 123) | | |
| Ds8.A.07-R | GCAACGGTTTACAAAGTCAGCGTCAACA (SEQ ID NO: 124) | | |
| Ds8.B.01-F | CGATCATGGCGTTCTTCATCCTCTATTTCTGTCT (SEQ ID NO: 125) | | |
| Ds8.B.01-R | TATTTTCTGCATTCAGTTCAGCAACGGCCA (SEQ ID NO: 126) | | |
| Ds8.B.02-F | ATACTGAAGCTGAAAATGAAATTCTGCTGGATGTCAC {SEQ ID NO: 127) | | |
| Ds8.B.02-R | GAGGGTGATCCGATCGAACAGCC (SEQ ID NO: 128) | | |
| Ds8.B.03-F | CTCGCATCTCAGGTGACCATAGACACC (SEQ ID NO: 129) | | |
| Ds8.B.03-R | AACGCTTCTGAAATGCCCCAGCT (SEQ ID NO: 130) | | |
| Ds8.B.04-F | GTTGCGTTGGGTAGTTATTTTACCGCATCG (SEQ ID NO: 131) | | |
| Ds8.B.04-R | TGCGTTACCGTTTCAGGTTGCAGG (SEQ ID NO: 132) | | |
| Ds8.B.05-F | GCACTTTTACCAGTCTTTCGGTCAGTTCACC (SEQ ID NO: 133) | | |
| Ds8.B.05-R | GGATCAATCGTCCAGACCTGCGC (SEQ ID NO: 134) | | |
| Ds8.B.06-F | TCCCGAGAAAAATACCGTCTCGCTGA (SEQ ID NO: 135) | | |
| Ds8.B.06-R | AGGGCTTTGCTGCTTTTTGTGAATCGG (SEQ ID NO: 136) | | |
| Ds8.B.07-F | CCTGGCCCCACTCAGTTATGATGAGT (SEQ ID NO: 137) | | |
| Ds8.B.07-R | CAACGGTTTACAAAGTCAGCGTCAACATCG (SEQ ID NO: 138) | | |
| | | Amplicon | |
| | Screening oligos | length | |
| pCC1Fos-T7- | GACTCACTATAGGGGAATTGTGAGCGGA (SEQ ID NO: 139) | 10.9 | kb |
| LacO_scrF | | | |
| Ds8_scrR1 | TCGGCCCCATACTGACGGGA (SEQ ID NO: 140) | | |
| Ds8_scrF2 | TTGCGGGGGCAGTCTGGAAG (SEQ ID NO: 141) | 10 | kb |
| Ds8_scrR2 | CGACGACGCCTGTGGGGAAA (SEQ ID NO: 142) | | |
| Ds8_scrF3 | TACAATCGCAGTGGCCGGGAG (SEQ ID NO: 143) | 9.9 | kb |
| pCC1Fos- | CAGCTCCGCGAAGTCGCTCT (SEQ ID NO: 144) | | |
| pSET_scrR | | | |
| M13R_SD | AGAGCTGCCAGGAAACAGCTATGAC (SEQ ID NO: 145) | 10.8 | kb |
| M13F_SD | ACGACGTTGTAAAACGACGGCCAG (SEQ ID NO: 146) | 7.8 | kb |

4.2.3 Partitioning of BGC for Golden Gate assembly. The BGC was identified from the Dickeya solani genome using antiSMASH 6.0 (Blin, et al, 2021). The reverse complement of the BGC and its flanking sequences were entered into a GC content calculator (Webgenetics, web). Regions, of at least 40 bp in length, with GC content above or below average for the cluster, were selected about every 4 kb. The BGC was entered into the NEBridge SplitSet™ Tool (NEB, NEBridge™ Ligase Fidelity, web) with the selected regions set as split regions, and BsaI-HFv2 or SapI master mix ligation conditions. Partition overhangs were entered into the NEBridge Ligase Fidelity Viewer™ and iterated for each set of ligation conditions to select the best assembly conditions for each enzyme. The part names, overhangs (sense strand), and number of internal restriction enzyme sites are listed in Table 4.2.

TABLE 4.2
Partitioned part names, overhangs, and
internal restriction enzyme sites.

| | | 5′ over- | 3′ over- | | | | |
|---|---|---|---|---|---|---|---|
| Name | Length | hang | bang | PaqCI | BbsI | BsaI | BsmBI |
| Ds8.A.01 | 3991 | ACGA | GAGT | | 1 | | 3 |
| Ds8.A.02 | 4021 | GAGT | CATC | | | | |
| Ds8.A.03 | 4117 | CATC | TATT | 1 | 2 | 1 | |
| Ds8.A.04 | 3691 | TATT | CTTT | 1 | 3 | 2 | |
| Ds8.A.05 | 4186 | CTTT | TTCT | 1 | 3 | | 1 |
| Ds8.A.06 | 3946 | TTCT | TGAG | | 1 | | |
| Ds8.A.07 | 4080 | TGAG | TTGC | | 2 | | 1 |

| | | 5′ over- | 3′ over- | | |
|---|---|---|---|---|---|
| Name | Length | hang | hang | EarI | LguI |
| Ds8.B.01 | 4020 | CGA | ATA | 1 | |
| Ds8.B.02 | 3985 | ATA | CTC | 3 | 1 |
| Ds8.B.03 | 4105 | CTC | GTT | 3 | |
| Ds8.B.04 | 3702 | GTT | GCA | | |
| Ds8.B.05 | 4156 | GCA | TCC | 2 | |
| Ds8.B.06 | 3956 | TCC | CCT | 3 | 1 |
| Ds8.B.07 | 4100 | CCT | TTG | 1 | 1 |

4.2.4 Amplification of parts. Fifty microliter PCR reactions were set up using Q5, or Q5U (for dUTP reactions). Parts containing internal restriction sites were amplified with dUTP, 5mCTP, or 7dGTP, as appropriate to the enzymes that would be used to clone those parts. The 5mCTP reactions used GC enhancer at 1×. Cycling conditions were: 98° C. for 30 minutes; 35 cycles at 98° C. for 10 seconds, or 100° C. for 30 seconds (5mCTP), 67° C. for 20 seconds (A.01, A.03) or 72° C. for 20 seconds, and 72° C. for 4 minutes; followed by a final extension of 72° C. for 2 minutes; and a hold at 4° C. The reactions were bead purified with 30 L of Omega Bio-tek Mag-Bind® TotalPure NGS beads (0.6X) to remove non-specific bands under 500 bp (Weitzman, 2018) and eluted with approximately 50 μL of EB buffer.

Failed PCR reactions were repeated using the originally amplified test amplicons as template and 25 PCR cycles. Annealing temperatures were adjusted, and for some amplifications 1M betaine or 1×GC enhancer was necessary for specific amplification. Gel purification was used when necessary to eliminate non-specific bands.

The non-tailed amplicons were phosphorylated as follows: 5 μL of NEB's T4 polynucleotide kinase (PNK) buffer was added to each, and then 10 μL of a master mix consisting of 1×T4 PNK buffer, 10 mM ATP, and 2 units of T4 PNK was mixed into each. The reactions were incubated for 2 hours at 37° C., 20 minutes at 65° C., and then held at 4° C. to bead purify again. For the amplicons that were gel purified 10 units of T4 PNK was used in 50 μL reactions with 1×T4 DNA Ligase buffer for 30 minutes at 37° C.

Amplicons were quantified with both Thermo's BR dsDNA Quant-iT kit or BR dsDNA Qubit kit, and a nanodrop. The nanodrop values were used for the 7dGTP amplicons, and the Quant-iT or Qubit values were used for the other amplicons.

4.2.5 Preparation of non-tailed amplicons for Golden Gate assembly. To test cloning of parts with methyltransferase-blocked restriction sites, approximately 100 fmoles of each restriction-site containing amplicon (Table 4.2) was methylated following the protocol in chapter 2, section 2.3.3. For each enzyme the non-tailed parts containing internal enzyme sites were combined into a single methylation reaction. For PaqCI and LguI, methylation was with GpC methyltransferase. For BsmBI, methylation was with CpG methyltransferase. For EarI, all of the parts were methylated together with EcoGII methyltransferase. Following heat kill of the methyltransferase, 100 fmoles of the remaining parts were added to the relevant reactions and the total DNA bead purified and quantified. For BsaI, Ds8.A.03 and Ds8.A.04 were individually treated with EcoGII methyltransferase and bead purified.

Based on the DNA quantifications, and assuming approximately equal amounts of each part, 20 fmoles of each part (140 fmoles of the total pooled parts) were A-tailed using NEB's NEBNext dA-Tailing Module following the protocol, and were then bead purified with 11 μL of EB buffer. For the non-tailed parts that were not pooled for treatment with a methyltransferase, approximately 20 fmoles of each part was pooled with the other parts and then A-tailed and bead purified.

Adapter ligations were set up using ˜1.4 pmoles (5×) of annealed TA adapters (Table 4.1), 10 μL of the A-tailing elution, and 11 μL of NEB's Blunt/TA master mix. Reactions were incubated for 1 hour at room temperature and then bead purified in 5 μL.

4.2.6 Golden Gate assembly of parts. The BsmBI PCR (dUTP) assembly substituted the unmodified Ds8.A.01_BsmBI part for the Ds8.A.01_BsmBI_dUTP part. The BbsI adapter (dUTP) and BsmBI adapter (dUTP) assemblies substituted the Ds8.A.01 unmodified part for the Ds8.A.01_dUTP part. The EarI adapter (dUTP) assembly substituted the Ds8.B.01 unmodified part for the Ds8.B.01_dUTP part. And the EarI PCR (dUTP) assembly substituted the Ds8.B.01_LguI_5mCTP part for the Ds8.B.01_LguI_dUTP part.

For the adapter-ligated reactions the entire purified reaction was assembled with 5 fmoles of vector. For the tailed amplicons 10 fmoles of each part was assembled with 5 fmoles of vector, except for the BsmBI PCR (dUTP) parts, which used 7 fmoles of each part, and the EarI PCR (dUTP) parts, which used 4.5 fmoles of each part.

The PaqCI and BsaI assemblies used the pCC1FosY-2LApR-PaqCI digested with PaqCI, QuickCIP-treated, and gel purified vector. The BsmBI assemblies used the same vector treated with CpG methyltransferase. The BbsI assembly used pINT-Chlor-FP-PaqCI digested with PaqCI, QuickCIP-treated, and gel purified vector. And the EarI and LguI assemblies used the pINT-Chlor-FP-LguI digested with LguI, QuickCIP-treated, and gel purified vector.

The PaqCI reactions used 5 units of PaqCI, an equal volume of activator, and 400 units of T4 DNA ligase. The BsaI reactions used 0.7 μL of NEB's NEBridge® Golden Gate Enzyme Mix (BsaI-HFv2). The BsmBI reactions used 0.7 μL of NEB's NEBridge® Golden Gate Enzyme Mix (BsmBI-v2). The BbsI reactions used 10 units of BbsI-HF and 400 units of T4 DNA ligase. The EarI reactions used 10 units of EarI and 400 units of T4 DNA ligase. And the LguI reactions used 0.5 μL of FastDigest LguI and 400 units of T4 DNA ligase. All reactions were performed in 1×NEB T4 DNA ligase buffer.

Cycling conditions were loosely based off NEB's recommendations for PaqCI assembly, with a final hold at 16° C. to promote ligation of internally digested parts prior to heat killing of the ligase: 30 cycles of 37° C. (42° C. for BsmBI-v2) for 5 minutes and 16° C. for 5 minutes; followed by 16° C. for one hour; 65° C. for 10 minutes; and a hold at 4° C.

The assembly reactions were PCR screened using the set5-barcoded screening oligos (table 4.1). Ten microliter PCR reactions used 0.1 μL of assembly reaction as template, 5 pmoles of each primer, 0.2 units of polymerase, and 1M betaine. The reactions were amplified with Phusion polymerase, and the dUTP reactions were repeated with Q5U polymerase. Cycling was 98° C. for 1 minute; 35 cycles of 98° C. for 10 seconds, 69° C. (Phusion) for 15 seconds, 72° C. for 10 minutes; followed by a final extension of 72° C. for 5 minutes; and a hold at 4° C. The Q5U cycling did not include the annealing step at 69° C.

4.2.7 Transformation of assemblies. One microliter of each assembly reaction was mixed with 40 μL of electrocompetent DH10β(Δung) cells (prepared in chapter 3). These were electroporated with a BTX ECM630 electroporation machine with HT-200 plate handler and a 2 mM gap electroporation plate. The parameters were 2.4 kV, 200Ω, and 25 μF. The reactions were outgrown in 1 mL of SOC for 2 hours at 30° C., and dilutions plated on LB bioassay trays with selection of Apramycin at 50 mg/L for the pCC1FosY-2LApR assemblies or Chloramphenicol at 25 mg/L.

For those assemblies with at least four pickable colonies, four colonies were picked into 150 μL cultures on Nunc 96-well, U-bottom plates, as indicated in table 4.3, along with colonies from the negative control reactions. The medium was LB+7.5% glycerol (Growcell MBLE-7970) with antibiotic as above. Cultures were grown overnight at 30° C.

4.2.8 Screening of colonies. To select colonies for full screening the glycerol stock plates were first screened with the Ds8.B.04 non-tailed cloning oligos (table 4.1). Template was generated by incubating 10 μL of overnight culture with 15 μL of water at 95° C. for 5 minutes. Half a microliter of this template was added to 10 μL PCR reactions using Q5 polymerase and 3 pmoles of primers. Cycling conditions were 98° C. for 30 seconds; 35 cycles of 98° C. for 10 seconds and 72° C. for 4 minutes; followed by a final extension of 72° C. for 2 minutes; and a hold at 4° C.

The positive colonies were screened with the full set of screening primers with the set1 and set2 barcodes. The assembly reactions which showed positive screening results were screened with the set1, set3, set4, and set5 barcoded primers for the positions that showed good screening results.

Twenty microliter PCR reactions were set up using Phusion, or Phusion U polymerase for the dUTP assembly reactions, with the GC buffer. Betaine was added to all reactions at 1M final concentration. For the assemblies, 0.2 μL of each reaction was used as template for PCR. For the colonies, 2 μL of the 95° C. lysed culture from above was used as template. Ten pmoles of each primer and dNTPs to the recommended concentration of 0.2 mM. Cycling conditions were 98° C. for 1 minute; 35 cycles of 98° C. for 10 seconds, 69° C. for 15 seconds, and 72° C. for 10 minutes; followed by a final extension at 72° C. for 5 minutes; and a hold at 4° C. A portion of the reactions were submitted for sequencing on a Pacific Biosciences Sequel IIe, deconvoluted with our barcode pipeline, and visualized with the Broad Institute's IGV.

### 4.3 Results

4.3.1 BGC selection and partitioning. Originally five ˜30 kb BGCs were selected as possible targets, 3 from balanced genomes, one from a GC-rich genome, and one from an AT-rich genome. Following the partitioning strategy the non-tailed primers were used to test amplification. All parts for both partition schemes, Ds8.A and Ds8.B, of this BGC (FIG. 22) and the AT-rich BGC amplified without PCR optimization. All but one part for each partition scheme amplified for the other two balanced BGCs, and 2/11 and 5/9 parts failed for the GC-rich genome.

The Ds8 BGC was selected to validate the modified nucleotide assembly strategy as it has a neutral 50% GC content which provides an informative balance between the adenosine and thymine base modification strategies and the cytosine and guanosine modification strategies used in this thesis. Additionally, its moderately large size and partitioning into 7 approximately equal length parts both allows demonstration of assembly efficacy on a moderately complex assembly using 8 overhang junctions, and removes part length as a source of variability.

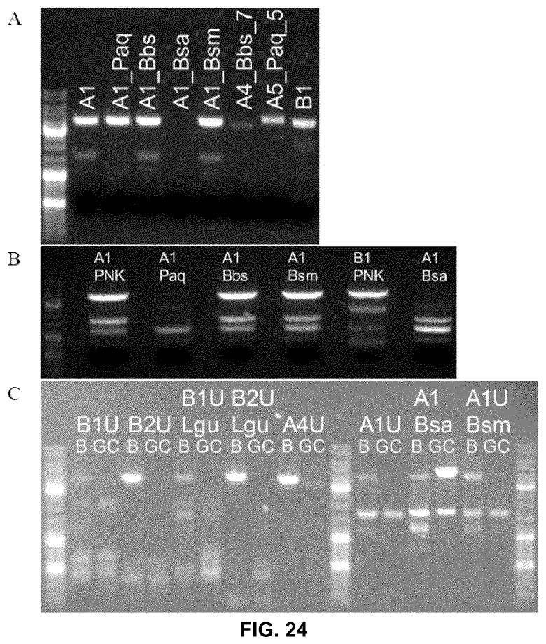

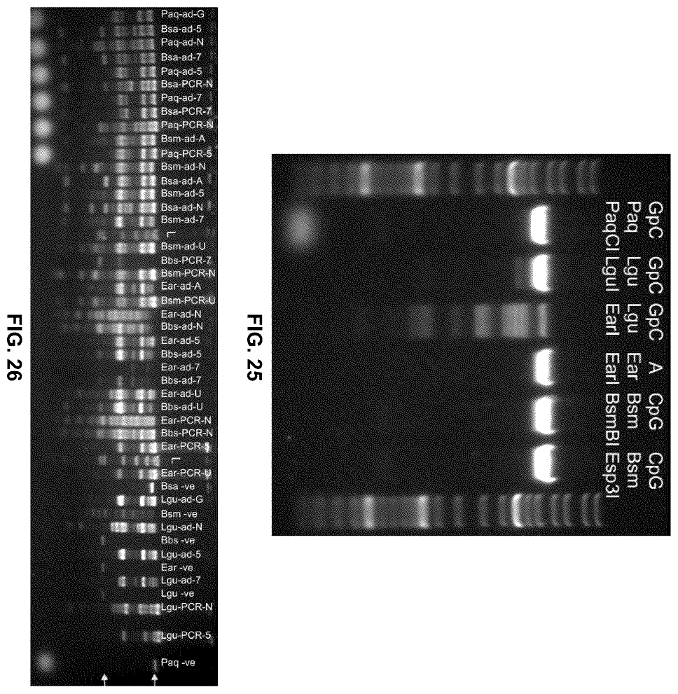

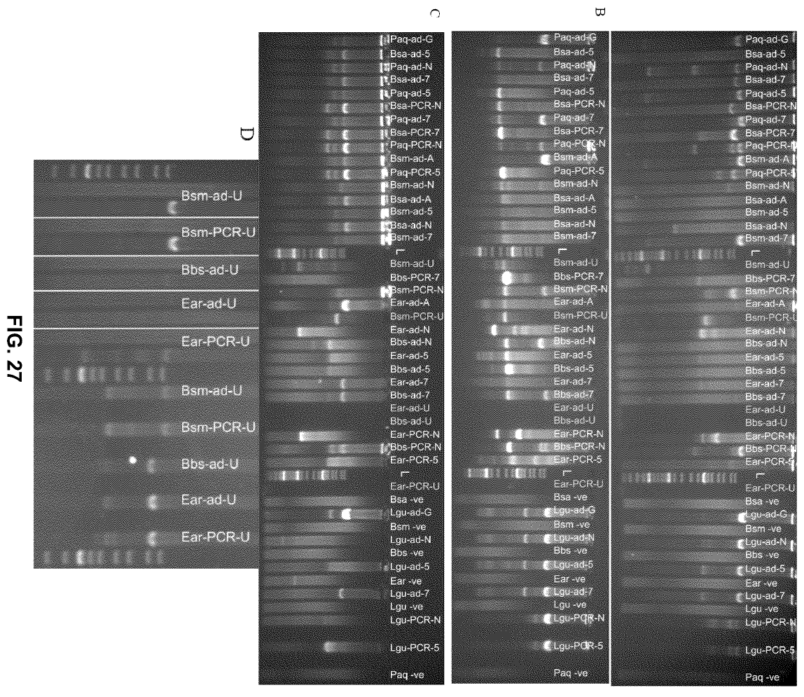

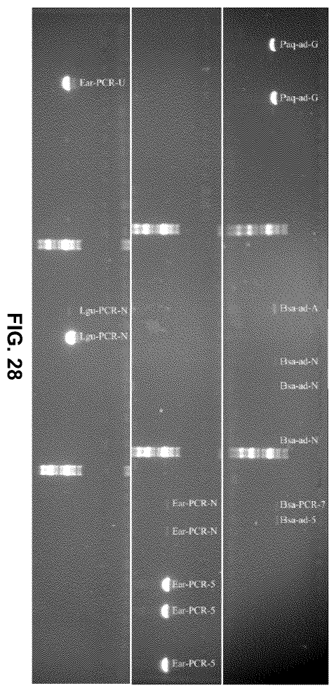

4.3.2 Amplification of Parts for Assembly. Successful PCR with non-tailed primers and regular dNTPs did not guarantee success with tailed primers, or PCR with the alternative nucleotides, at least on the first round (FIG. 23), even when the original PCR with non-tailed primers was robust. Additionally, PCR with tailed primers led in some cases to additional non-specific bands. Two rounds of minor PCR optimization successfully amplified all but 4 dUTP parts which were abandoned for insufficient yield of the specific band (FIG. 24), and substituted with their dNTP or 5mCTP equivalent.

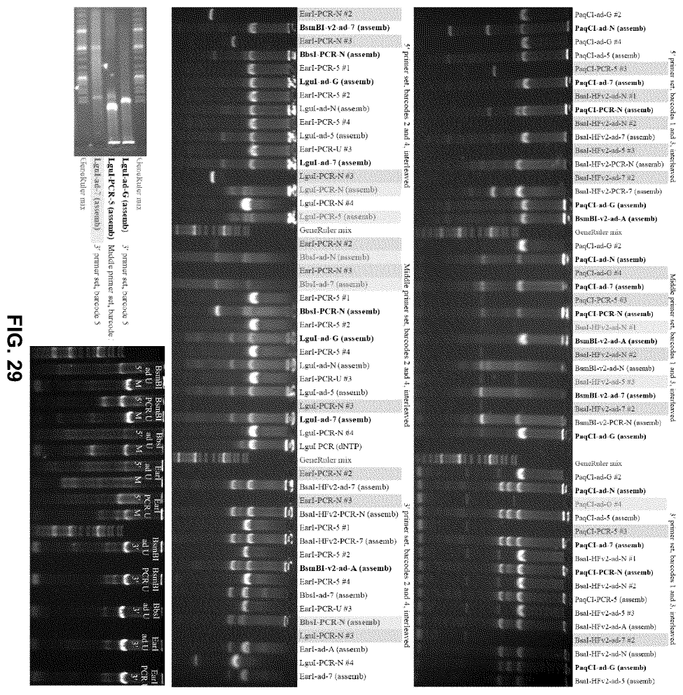

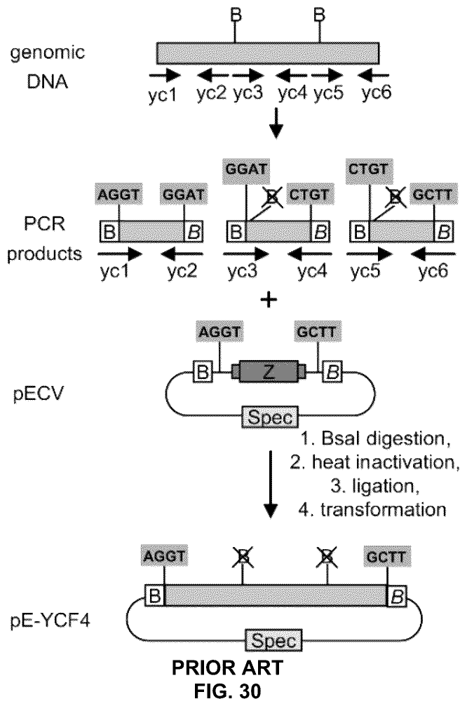

4.3.3 Protection of Parts by Nucleotide Modification. As seen in FIG. 25 in the LguI digest and FIG. 26 in the BsmBI vector only control reaction, methylation of parts did not fully protect those parts from digestion. This is a verification of chapter 2 table 2.4 which showed that the methylation was only inhibitory to digestion, instead of fully blocking digestion, for GpC methylation with respect to LguI and CpG methylation with respect to BsmBI-v2.

4.3.4 Direct Validation of Assembly Reactions. In FIG. 26, half of the Golden Gate assembly reactions were run out on a 0.7% agarose gel and post-stained with Sybr Gold. The Golden Gate assembly reactions using unmodified parts universally showed a greater number of bands from internal digestion by the restriction enzyme and possible misassembly of parts by the ligase, when compared to the modified reactions.

The BsaI-HFv2 assemblies mostly showed bands at around 500 bp and 2.4 kb, even in reactions in which they should have been protected. The BsmBI-v2 assemblies all showed a lower top band than the surrounding BsaI-HFv2 and PaqCI assemblies, which may demonstrate incomplete assembly due to digestion of the vector backbone. The EarI and LguI assemblies showed absence of the vector band, indicating successful assembly has begun, while the BbsI-HF assemblies still showed vector present, indicating either slower assembly (adapter assemblies) or unsuccessful assembly (PCR 7dG).

The BsmBI vector only control reaction shows partial digestion of CpG methylated vector DNA, which is present but less evident in the assembly reactions. For all enzymes the unmodified reactions show laddering from digestion of parts.

The assembly reactions in FIG. 26 were also PCR screened with the set5 barcoded primers that walk across the entire assembly. This was done to better identify which assembly reactions were able to properly assemble the various parts. This is not a perfect analysis, as at least one assembly, EarI PCR (5mCTP), which gave colonies with full-length inserts, did not show a full-length band for at least one region of the assembly reaction screen.

In FIGS. 27 and 29, the BsmBI, BsaI, BbsI, and EarI reactions mostly appeared to have performed poorly. There are some exceptions. The most successful assemblies, with full-length bands across all three screening PCRs, were: For PaqCI: The primer-tailed PCR assembly with regular dNTPs, and the adapter-ligated assemblies with GpC methylation, unmodified nucleotides, and the 7dGTP modified parts. For BsmBI-v2: The primer-tailed PCR assembly with dUTP, and the adapter-ligated assemblies with EcoGII methylation, 7dGTP modified parts, and dUTP modified parts. For BbsI-HF: The primer-tailed PCR assembly with regular dNTPs, and the adapter-ligated 7dGTP modified parts, and dUTP modified parts. For EarI: The primer-tailed PCR assembly with dUTP, and the adapter-ligated dUTP modified parts. For LguI: The adapter-ligated assemblies with GpC methylation, and the 7dGTP modified parts.

4.3.5 Golden Gate assemblies. DH10β(Δung) electrocompetent cells were used for all assembly reactions to minimize variability in colony counts based on the competent cells, to make resistance approximately equal for all wells across the multi-well electroporation plate, and to make transformation setup generally easier and less prone to error.

FIGS. 26, 27 and 29 show that a number of the assemblies appear to have worked. The individual colony screening results from FIG. 28 showed that only a few of them had picked colonies with the middle insert present. These results may have been much better had the assembly protocol used more cycles, and had the vector been linearized for cloning by the restriction enzyme used for assembly.

FIG. 28, and the full-length screening PCRs in FIG. 29, showed poor assembly efficiency for many of the strategies. The PaqCI adapter (GpC), the EarI PCR (5mCTP), and the EarI PCR (dUTP) had full length screening results from one, three, and one transformation colonies, respectively. For the EarI PCR (5mCTP) assembly, the 3 out of 4 full length colonies were from a total of 6 colonies on the plate (table 4.3). All five of these colonies were one of the seven colonies with intense bands on the original Ds8.B.4 screening gel (FIG. 28).

List of abbreviations: Paq=PaqCI, Bsa=BsaI-HFv2, Ear=EarI, Lgu=LguI; ad=adapter-ligated, PCR=primer-tailed; G=GpC methylated, A=EcoGII methylated, 5=5mCTP modified, N=unmodified, 7=7dGTP modified, U=dUTP modified

TABLE 4.3
Colony counts of the BGC assembly transformations. 1/10^{th} of
the outgrowth was plated. This shows the colony counts (CFUs)
and picked colonies for each assembly transformation.

| Assembly | CFUs | picked | Assembly | CFUs | picked |
|---|---|---|---|---|---|
| PaqCI adapters (GpC) | 44 | 4 | BbsI adapters (dNTP) | 22 | 4 |
| PaqCI adapters (dNTP) | 32 | 4 | BbsI adapters (5mC) | 12 | 4 |
| PaqCI adapters (5mC) | 24 | 4 | BbsI adapters (7dG) | 17 | 4 |
| PaqCI adapters (7dG) | 10 | 4 | BbsI adapters (dUTP) | 34 | 4 |
| PaqCI PCR (dNTP) | ~100 | 4 | BbsI PCR (dNTP) | 28 | 4 |
| PaqCI PCR (5mC) | 38 | 4 | BbsI PCR (7dG) | 18 | 4 |
| PaqCI -ve | 1 | 1 | BbsI -ve | 3 | 1 |
| BsaI adapters (EcoGII) | 15 | 4 | EarI adapters (EcoGII) | 9 | 4 |
| BsaI adapters (dNTP) | 24 | 4 | EarI adapters (dNTP) | lots | 4 |
| BsaI adapters (5mC) | 28 | 4 | EarI adapters (5mC) | 4 | 4 |
| BsaI adapters (7dG) | 4 | 4 | EarI adapters (7dG) | 1 | 0 |
| BsaI PCR (dNTP) | 14 | 4 | EarI adapters (dUTP) | lots | 4 |
| BsaI PCR (7dG) | 10 | 4 | EarI PCR (dNTP) | lots | 4 |
| BsaI -ve | 2 | 2 | EarI PCR (5mC) | 6 | 4 |
| BsmBI adapters (EcoGII) | 13 | 4 | EarI PCR (dUTP) | ~40 | 4 |
| BsmBI adapters (dNTP) | 1 | 0 | EarI -ve | 2 | 1 |
| BsmBI adapters (5mC) | 8 | 4 | LguI adapters (GpC) | 22 | 4 |
| BsmBI adapters (7dG) | 1 | 0 | LguI adapters (dNTP) | lots | 4 |
| BsmBI adapters (dUTP) | 8 | 4 | LguI adapters (5mC) | 9 | 4 |
| BsmBI PCR (dNTP) | 1 | 0 | LguI adapters (7dG) | 1 | 0 |
| BsmBI PCR (dUTP) | 0 | 0 | LguI PCR (dNTP) | lots | 4 |
| BsmBI -ve | 0 | 0 | LguI PCR (5mC) | 5 | 4 |
| | | | LeuI -ve | | 0 |

4.3.6 Sequencing results. Only one of the five full-length colonies was sequence perfect, which is colony #3 of the EarI PCR (dUTP) transformation that used a 5mCTP amplicon as part 1. Another had a single C>T base change, which is colony #2 of the PaqCI adapter GpC transformation. The remaining three EarI colonies, all from the 5mCTP PCR transformation, had a total of 17 single base mutations between them, 15 of them are G>A or C>T conversions, one has a 4C>3C shrinkage of a homopolymer run and a single T>G conversion (Appendix F). This is an average of one G:C>A:T conversion per 5.6 kb, or one 5mC>T conversion per every 2.8 thousand 5mC nucleotides.

PCR and sequencing of assembly reactions showed mixed or full deletions in some assemblies (Appendices G and H). A particularly noteworthy deletion was a complete deletion of part Ds8.A.07 from a portion of reads of all of the pCC1FosY-2LApR assemblies, with the four PaqCI adapter based assemblies showing the most reads with deletions. Full-length sequences for some of the 7dGTP adapter assemblies showed that Type IIS restriction enzymes could generally cleave 7dG modified DNA from non-modified recognition sequences, and that T4 DNA ligase can ligate these ends together.

Final results show that successful assemblies were performed from both adapter-ligated parts and from parts amplified with restriction enzyme tails.

### 4.4 Discussion

The Golden Gate assemblies were not optimized in any way. Neither in enzyme amounts, cycling conditions, or DNA amounts. Due to the high-throughput nature of this test, the desired DNA fragments were not gel purified, and direct sequencing results of the assemblies showed that sometimes shorter, truncated amplicons carried through into assembly.

The primary vector, pCC1FosY-2LApR, unfortunately was only optimized for two of the enzymes, PaqCI and BsaI, and only linearized by PaqCI. This is unfortunate as a final digestion step in Golden Gate assembly can decrease the amount of background colonies. CpG methylation was used to block internal BsmBI sites, but since CpG methylation only inhibits digestion with BsmBI-v2, this did not work as well as hoped. The secondary vector, pINT-Chlor, used a pUC origin of replication. This is a high-copy origin of replication that is recommended for only smaller inserts. Growth of assemblies was performed at 30° C. at all steps in order to lower plasmid copy number, but even so the size of the BGC would push the plasmid size to about 30 kb, which is outside of the recommended cloning range of the pUC replication origin.

When using methyltransferases to protect parts with internal restriction enzyme sites I chose to only methylate the specific parts with those internal sites. Likewise, I only PCR amplified parts with modified nucleotides when they needed protection. This maximized part reuse between assemblies, and also minimized the effect of modification on assembly efficiency and mutations. This did require treating parts differently, which could be tedious during high-throughput assemblies, and could also lead to variation in part quality or quantity. If desired this can be simplified by modifying all parts the same way. If this is done, depending on the assembly strategy, tradeoffs may have to be made. Modification of all parts with CpG or GpC methyltransferases will probably have no effect outside of GC-rich assemblies. PCR of all parts with dUTP likewise should have no effect outside of possibly reduced part quantities. PCR of all parts with 5mCTP will likely reduce colony counts, and will increase the chance of C>T conversions. Methylation of all parts with EcoGII methyltransferase will likely reduce colony counts in all but the most GC-rich assemblies.

Assembly screening and sequencing showed that many of the assembly reactions worked well. This screening was unfortunately incomplete, and sequencing of full-length colonies showed that some of the assemblies that didn't show full screening over all parts of the assembly were effective (specifically the EarI PCR 5mCTP assembly). The empirical results of this chapter should be used to guide selection of Type IIS enzyme and DNA modification strategies, but the results of chapter 2, as well as theoretical strategies based on the nucleotide content of the restriction enzymes and the blocking ability of the various DNA modifications on these enzymes, should still be considered for further testing.

From these results the PaqCI adapter-based cloning using GpC methylated DNA worked well, and the EarI primer-based cloning using 5mCTP or dUTP amplified DNA worked very well.

A number of G:C>A:T mutations were seen in the EarI PCR 5mCTP assemblies, for which all parts were amplified with 5mCTP PCR, at a rate of about 1 conversion for every 2.8 thousand 5-methyl-cytosines for the PCR conditions used here. Deaminated cytosines are chemically identical to uracils, which will be converted to thymines in an Ung deficient bacterial strain. However, more importantly, deaminated 5-methyl-cytosines are already chemically identical to thymine, and so will be read as thymines regardless of cloning strain.

The two single nucleotide mutations in the BsaI adapter 5mCTP clone #3 were also G:A>C:T conversions. One of these mutations was in the 5mCTP amplified part 4, and was present in most reads. The other mutation was in the dNTP amplified part 5, and was present in only a minority of reads. The single G:A>C:T conversion in the PaqCI adapter GpC clone #4 was in the dNTP amplified part 1, with none in the GpC methylated parts. And PaqCI adapter GpC clone #2 had a single A:T>G:C conversion in a dNTP amplified part. The EarI PCR dUTP clone had no mutations.

These results indicate that the vast majority of these G:A>C:T conversions are likely a result of the deamination of 5-methyl-cytosine, while only a few may be the result of random PCR mutations, in vivo mutations, or C>U>T conversion of deaminated, non-methylated cytosines by the DH10β(Δung) cloning strain.

The major cause of cytosine deamination is the high temperatures of PCR. At neutral pH and 95° C. 5-methyl-cytosine in DNA deaminates at about 4 times the rate of cytosine (Lindahl, Nyberg, 1974). In this chapter PCR cycles and conditions were not optimized, but were permissive. For a 4 kb amplicon, most high-fidelity polymerases recommend a 1 to 2 minute extension time at 72° C. This cautions against amplifying very long parts with 5mCTP, without some way to prevent the polymerized 5-methyl-cytosines from deaminating. If amplifying AT-rich DNA the recommended lower 2-step annealing and extension step may help protect against deamination, though possibly countered by the increased extension time. Alternatively, 2-step amplification with 4mCTP at lower temperatures could be revisited, as N⁴-methyl-cytosine has a lower deamination rate than even cytosine (Ehrlich, 1986).

Typically, the high-fidelity PCR enzymes recommend 25 cycles, and often fewer cycles can be used, though this requires adequate template. In this chapter, 35 cycles were used, increasing the time at elevated temperatures by 40%. Also, while a higher denaturing temperature or longer denaturing time is required for 5mCTP amplification, 100° C. for 30 seconds may be overkill. This time can probably be shortened, and the temperature may be able to be decreased by using additives. In chapter 2, Thermo's SuperFi II showed robust amplification with 5mCTP without additives, and may be the best candidate to test.

This BGC had a 50% GC content. As such, PCR with dUTP or 5mCTP would modify an equal number of bases. When amplifying parts with more biased GC content, dUTP may be better than 5mCTP for high-GC targets, and vice versa for low-GC targets. Though recognizing that dUTP amplification of high-GC targets requires cloning into an Ung deficient strain, which may result in C>U>T conversion mutations. While C>U>T conversions seem to be rare based on these results, a higher GC assembly will have more opportunity for them. For this purpose, it would be especially important to minimize the time the construct is in the Ung deficient strain. A possible protocol would be to outgrow the transformation briefly, add antibiotic selection to the outgrowth and let it grow for an hour or two in order to allow plasmid replication and the replacing of uracils with thymines, then plasmid prepping the entire outgrowth and transforming it into an Ung proficient strain for plating and colony selection.

Most of the 7dGTP adapter-based assembly reactions seemed to work well. Yet they yielded fewer colonies, of which none of the picked ones screened positively. I'm still curious whether partially 7dG-substituted DNA would clone, but it seems best to abandon this nucleotide for cloning purposes without further research.

For the adapter assemblies there is a possibility that incomplete extension during PCR could create its own overhang without need for restriction digestion from a ligated adapter. There are assembly techniques that use incomplete extension for cloning, those these typically use much longer homologous regions than 3 or 4 bases. It is unlikely that incomplete extension would lead to many clones, as there would be no assurance of exactly the 3 or 4 base overhangs needed for ligation.

An interesting result was the partial part 7 deletion in the pCC1FosY-2LApR assemblies. The sequence contexts for the Golden Gate overhangs were aTGAGa for the parts 6/7 junction, and gTTGCg for the part 7/vector junction. The 5′ overhangs left after digestion would be tACTC on the complementary strand of the 6/7 junction, and TTGCg on the 7/vector junction. All sequencing reads show TGAG for the junction, which implies that only one strand ligated, and that this strand is the complementary (bottom) strand. Ligation appears to have been with the TGCg of the 6/7 junction, instead of the TTGC. I do not know how this happened, though can speculate that extended restriction digest or dephosphorylation of the vector may have allowed removal of the terminal base of either of the overhangs thus promoting splicing. I also do not know if this would have resulted in truncated clones had more positive colonies been identified. Alternatively, it's possible that a primer dimer artifact created this junction, but the bead purification of the parts should have eliminated any primer dimers short enough to do so.

This part 7 sequencing does identify a means of detecting, and even quantifying, single-strand splice-junctions from Golden Gate assemblies. PCR can be done on the assemblies to show which strand, and thus overhang, spliced the junction. For validating junctions, assemblies can be done with one strand containing a 5′ phosphate, the other strand phosphate-free, and a digestion with Lambda exonuclease prior to the PCR step to preferentially eliminate the strand with the 5′ phosphate. PCR with one cycle would be sufficient to synthesize the second strand without creating numerical bias, and elimination of one strand of the template with Lambda exonuclease would prevent duplication of fully-ligated junctions compared to single-strand ligated junctions.

### CONCLUSION

Not knowing beforehand which combinations of nucleotide modification, assembly strategy, and Type IIS enzyme would work I tested as many as possible. From REBASE and general knowledge of the Type IIS restriction enzyme recognition sites there were reasons to assume that some combinations of restriction enzyme and modification strategy would not work. This allowed using these combinations as presumptive negative controls, though results did show a few surprises.

Golden Gate assembly, and restriction enzyme assembly in general, are mature technologies. For any given researcher there may be a lot of sunk costs requiring the use of specific restriction enzymes or cloning strains. By testing as many restriction enzymes, modification types, and assembly strategy combinations as possible I have hopefully made this work more useful to a broader number of researchers. But if options exist there are a number of preferences.

The protocols used in this thesis were not optimized in any way, and should not be considered authoritative. Theoretically primer-borne restriction sites with nucleotide modifications incorporated with PCR is the most inexpensive, straight-forward, and stoichiometrically controllable assembly strategy, but secondary structure or mis-priming from the primer tails may occasionally cause issues. Alternatively, adapter-ligation allows minimal modification of DNA through methylation of cytosine or adenosine residues, and allows the use of sequence verified, pre-cloned DNA parts, as seen in the MASTER paper that inspired this thesis. For balanced GC-content genomes dUTP PCR is likely to be higher fidelity than 5mCTP PCR, though it requires special polymerases and cloning strains. When incorporating enzyme sites into primers, use more than two bases before the enzyme recognition site to promote complete digestion. In general, the least amount of DNA modification is best. PCR with dUTP comes with increased risk of C>U>T conversions in the required Δung cloning strain, while PCR with 5mCTP comes with an even greater risk of 5mC>T conversions during the high temperatures of PCR. Except in very high-GC parts, CpG and GpC methyltransferases methylate fewer bases than EcoGII methyltransferase.

For PCR with dUTP and tailed-primer sites the enzymes BsmBI-v2, Esp3I, and EarI are recommended. For PCR with 5mCTP and tailed primer sites PaqCI, BsmBI-v2, EarI, and LguI or its isoschizomer SapI are recommended. For methyltransferase protection and adapter-ligation, GpC protection and the enzymes PaqCI or SapI are recommended. If BsaI has to be used, adapter ligation of 5mCTP amplified DNA or EcoGII methylated DNA is required. For BbsI adapter ligation is required, but dUTP amplified DNA can be used in addition to 5mCTP amplified or EcoGII methylated DNA.

For larger assemblies, PCR with dUTP and cloning into a Δung cloning strain is recommended, or GpC methyltransferase protection and cloning with PaqCI or SapI, this is especially the case if significant lengths of the assembly need protection. If 5mCTP PCR is used, it is important to optimize cycling conditions to limit the amount of time at high temperatures. This can be done by using more template to reduce the number of cycles, using recommended extension times for the polymerase, and using the lowest denaturing time and temperature required for specific amplification. Though additives may somewhat decrease polymerase fidelity, if they decrease time at high temperatures this would be beneficial for 5mCTP PCR.

GC content: For high-GC assemblies, dUTP PCR would be greatly preferred over 5mCTP PCR, not only because of the lower possibility of mutation in dUTP PCR, but because of the higher denaturing temperatures and times required for 5mCTP PCR. Though this needs to be balanced with the potential for C>U>T conversions from the Δung cloning strain. For a 75% GC content, EcoGII methylation would leave 12.5% of bases methylated, while CpG or GpC methylation would leave an average of 14% of bases methylated, not counting off-target effects of GpC methyltransferase. At these percentages, EcoGII might result in more colonies and have the benefit of blocking digestion with more enzymes. If dUTP PCR will be used for very large high-GC assemblies, minimizing the time in the Δung cloning strain becomes more important. A protocol which plasmid preps the Δung transformation outgrowth after an extended outgrowth period, and then transforms the prep into a regular cloning strain may be beneficial. An extra hour or two of outgrowth, with or without added selection, should be adequate to convert uracil containing plasmids to thymine containing plasmids.

For low-GC assemblies 5mCTP PCR may be preferred given the lower yields of dUTP PCR, though 5mC>T conversion still requires minimizing PCR time at high temperatures. For PCR of low-GC parts a low 2-step annealing/extension step is generally recommended; the use of 5mCTP PCR might allow a higher annealing/extension step and consequently shorter cycle times. Alternatively, adapter ligation of CpG or GpC methylated DNA result in even less modification to the DNA.

Protection of Vectors: Assemblies with the least background have the vector digested by the same enzyme which cuts the inserts, as this allows one-pot assembly with a final linearization step. If a different enzyme is needed to linearize the vector than the parts it should first be determined whether the parts are protected against digestion by this enzyme. If they are protected, then a one-pot reaction with both enzymes is preferred.

The vector also needs to not be digested internally by the enzyme which cuts the inserts. If these enzyme sites exist in the vector, then two approaches are possible: The standard one-pot Golden Gate that ends on a ligation step to re-ligate the vector, or pre-digestion of the vector with its enzyme (if required) and protection of the vector backbone from digestion with the other enzyme.

A one-pot Golden Gate reaction can promote misassemblies between vector and insert overhangs, or vector and vector overhangs, if there's even a little cross-talk between overhangs. Additionally, even with no cross-talk, digestion and religation of the vector backbone could promote concatemers.

Of the backbone modifications tested, CpG and GpC methyltransferase protection showed no inhibition of colony counts. CpG methylation was shown to provide some protection against digestion with SapI, Esp3I, or BsmBI-v2, but should be used with caution as this protection is not complete. GpC methylation was shown to provide complete protection against digestion with PaqCI and its isoschizomer AarI, and SapI.

Combinatorials and Libraries: For combinatorial and library assemblies three things are important: total number of colonies, proportion of correct colonies, and equal representation of the parts. While highly methylated DNA was shown to increase the ratio of correct colonies, it also reduced total colony count, and could have an effect on equal representation of parts depending on the degree of methylation for each part. If a significant fraction of the assembly needs to be protected, PCR with dUTP, or adapter ligation of dUTP, CpG, or GpC protected parts would generally cause the least bias while still generating ample correct colonies.

Protocol optimization: The protocols I used for adapter ligation required up to three additional purification steps. DNA tailing and adapter ligation is used frequently for current generation sequencing technologies, and protocols or buffers have been optimized to reduce the number of purification steps. Depending on buffer compatibility, it might be possible to allow sequential methylation, A-tailing, and adapter ligation with only one or two purification steps.

Nucleotides: Amplification generated less DNA, at least full-length DNA, with nucleotide substitution. This was especially noticeable with dUTP amplification. If partial substitution of nucleotides is going to be tested, the degree of nucleotide substitution during PCR with dUTP/dTTP mixes or dCTP/5mCTP mixes will need to be empirically determined on a variety of targets.

In PCR N⁴-methyl-cytosine has significant benefits over 5-methyl-cytosine, or even plain cytosine, with respect to deamination and consequent conversion to thymine. It also lowers melting temperature, which may be of benefit in the PCR of high-GC targets. In this study 4mCTP PCR was only successful with one high-fidelity polymerase, and required a very low temperature 2-step cycle. PCR with 4mCTP may be worth revisiting and optimizing.

7dGTP showed promising results for amplification, and completely blocked internal sites of all tested Type IIS enzymes. However, it showed the poorest cloning results of all tested modifications. In sanger sequencing 7dGTP is known to allow synthesis of some high-GC regions. For some purposes it may be worth testing partial substitution of dGTP with 7dGTP in PCR. Additionally, literature searches to see if particular DNA glycosylases or endonucleases present in the DH10β genome may prevent cloning with 7-deaza-guanosine through error repair. If error repair is responsible for the inhibition of colony formation of 7-deaza-guanosine containing DNA, then knocking out the responsible gene may allow cloning in the same manner as knocking out the ung coding sequence allowed cloning of uracil containing DNA.

Primer-borne cloning completely failed for BbsI sites as PCR with dUTP completely modifies the primer-borne site, and PCR with 5mCTP modified two of the three cytosines of the recognition site, which was sufficient to block assembly. An adenosine or guanosine analog that can be used to PCR with, and that doesn't impede colony counts, would be a valuable option.

Partial substitution: For vectors with internal restriction enzyme sites only protection with GpC methyltransferase was shown to work well, thus limiting the enzymes which could be protected against. PCR of vectors with fully substituted dUTP, 5mCTP, or methylation with EcoGII to as close to completion as feasible, greatly reduced colony count. It's likely that PCR of the vector with partial uracil or 5mCTP substitution, or partial EcoGII methylation of pre-linearized vector, would have less of an effect on colony count. A simple restriction digestion and gel purification to remove incompletely protected DNA may allow protection against all of the Type IIS restriction enzymes. For the PCR amplified vector, primer-borne LguI/SapI, EarI, BsmBI-v2, or PaqCI/AarI sites could be used to free the cloning overhangs for Golden Gate assembly.

For 5mCTP PCR and EcoGII methylation, partial substitution for inserts may also be worth investigating, though would require two rounds of part purification, at least one of which is gel purification, prior to assembly.

Secondary structure: A real benefit of Golden Gate assembly over chewback assembly (e.g. In-Fusion, Gibson) for scarless assembly of parts is that secondary structure near the cloning junctions is much less problematic for proper assembly. It can still be problematic for PCR when the secondary structure encompasses the primer binding site. PCR optimizations or additives may overcome this secondary structure. And while very long primers can be used to fully bridge the region of secondary structure with a 3′ anchor in a region without secondary structure, these primers are currently much more expensive than typical PCR primers, and have an increased possibility of non-specific amplification or primer synthesis errors.

The restriction enzymes BtgZI and BsmFI have 10 fully degenerate nucleotide spacings between their recognition sites and cleavage sites. This could allow 5′ anchoring outside of the secondary structure, either with primer-borne sites, or from ligated adapters.

### Appendix A: List of Abbreviations Used Herein:

Nucleotide analogs, referring to the incorporated nucleotide, or the free triphosphate, depending on context: deoxyuracil: uracil, dUTP, U; 4-Thio-deoxythymine: 4-Thio-dTTP, 4tdTTP, 4tdT; 5-Hydroxymethyl-deoxyuracil: 5-Hydroxymethyl-dUTP, 5homUTP, 5homU; N⁶methyl-deoxyadenosine: N⁶methyl-dATP, 6mATP, 6 mA: 7-Deaza-2′-deoxyadenosine: 7-deaza-dATP, 7dATP, 7dA: N⁴methyl-deoxycytosine: N⁴-methyl-cytosine, N⁴methyl-dCTP, 4mCTP, 4mC: 5-methyl-deoxycytosine: 5-methyl-cytosine, 5-methyl-dCTP, 5mCTP, 5mC: 7-Deaza-2′-deoxyguanosine: 7-deaza-guanosine, 7-deaza-dGTP, 7dGTP, 7dG.

Methyltransferase-modified DNA: EcoGII modified DNA: EcoGII, A: CpG modified DNA: CpG, C; GpC modified DNA: GpC, G.

Polymerases and buffers: HF=High-fidelity buffer; GC=GC buffer or the Q5 GC enhancer additive; B=Betaine additive; Polymerases are typically abbreviated by their first one or two letters.

Appendices B-I are described in U.S. Provisional Patent Application Ser. No. 63/516,411, filed Jul. 28, 2023, which are hereby incorporated by reference.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

All cited references are hereby each specifically incorporated by reference in their entireties.

What is claimed is:

1. A method for one-pot Golden Gate cloning, or conventional two-pot restriction enzyme cloning, regardless of internal restriction sites, comprising: using modified nucleotides during PCR of a DNA, or through post-hoc enzymatic modification of already existing DNA, such that the use of the modified nucleotides blocks the internal restriction sites while leaving the flanking restriction sites unblocked.

2. A kit comprising one or more compositions, components, enzymes, primers, oligos, or the like as described herein, sufficient for carrying out the method of claim 1.


SEQUENCE LISTING (146 sequences)

SEQ ID NO: 1 - DNA, 48 bp, Escherichia coli
  source 1..48: mol_type = genomic DNA
gcttgaagattcgcagcagagcgagatggctaacgaattaacctggca

SEQ ID NO: 2 - DNA, 714 bp, Escherichia coli
  source 1..714: mol_type = genomic DNA
caggagagcgagatggctaacgaattaacctggcatgacgtgctggctgaagagaagcagcaaccctattttcttaatacccttcagaccgtcgccagcgagcggcagtccggcgtcactatctacccaccacaaaaagatgtctttaacgcgttccgctttacagagttgggtgacgttaaagtggtgattctcggccaggatccttatcacggaccgggacaggcgcatggtctggcattttccgttcgtcccggcattgccattcctccgtcattattgaatatgtataaagagctggaaaatactattccgggcttcacccgccctaatcatggttatcttgaaagctgggcgcgtcagggcgttctgctactcaatactgtgttgacggtacgcgcaggtcaggcgcattcccacgccagcctcggctgggaaaccttcaccgataaagtgatcagcctgattaaccagcatcgcgaaggcgtggtgtttttgttgtggggatcgcatgcgcaaaagaaaggggcgattatagataagcaacgccatcatgtactgaaagcaccgcatccgtcgccgctttcggcgcatcgtggattctttggctgcaaccattttgtgctggcaaatcagtggctggaacaacgtggcgagacgccgattgactggatgccagtattaccggcagagagtgagtaaatttgcggggaa

SEQ ID NO: 3 - DNA, 33 bp, Synthetic construct
  source 1..33: mol_type = other DNA
ttgctcttctccatggctaattcccatgtcagc

SEQ ID NO: 4 - DNA, 31 bp, Synthetic construct
  source 1..31: mol_type = other DNA
ttcgtctcttggcaaagcctcgcaatccagt

SEQ ID NO: 5 - DNA, 24 bp, Synthetic construct
  source 1..24: mol_type = other DNA
tgccacctgacgtctaagaaacca

SEQ ID NO: 6 - DNA, 22 bp, Synthetic construct
  source 1..22: mol_type = other DNA
ggcacttttcggggaaatgtgc

SEQ ID NO: 7 - DNA, 36 bp, Synthetic construct
  source 1..36: mol_type = other DNA
ttgaagaccccctatagtgagtcgtattaatttcgc

SEQ ID NO: 8 - DNA, 33 bp, Synthetic construct
  source 1..33: mol_type = other DNA
ttgaagactataggggaattgtgagcggataac

SEQ ID NO: 9 - DNA, 37 bp, Synthetic construct
  source 1..37: mol_type = other DNA
ttcgtctctcctatagtgagtcgtattaatttcgccc

SEQ ID NO: 10 - DNA, 32 bp, Synthetic construct
  source 1..32: mol_type = other DNA
ttcgtctcttaggggaattgtgagcggataac

SEQ ID NO: 11 - DNA, 38 bp, Synthetic construct
  source 1..38: mol_type = other DNA
ttgctcttctcctatagtgagtcgtattaatttcgccc

SEQ ID NO: 12 - DNA, 32 bp, Synthetic construct
  source 1..32: mol_type = other DNA
ttgctcttctaggggaattgtgagcggataac

SEQ ID NO: 13 - DNA, 37 bp, Synthetic construct
  source 1..37: mol_type = other DNA
ttctcttctcctatagtgagtcgtattaatttcgccc

SEQ ID NO: 14 - DNA, 31 bp, Synthetic construct
  source 1..31: mol_type = other DNA
ttctcttctaggggaattgtgagcggataac

SEQ ID NO: 15 - DNA, 37 bp, Synthetic construct
  source 1..37: mol_type = other DNA
ttggtctctcctatagtgagtcgtattaatttcgccc

SEQ ID NO: 16 - DNA, 32 bp, Synthetic construct
  source 1..32: mol_type = other DNA
ttggtctcttaggggaattgtgagcggataac

SEQ ID NO: 17 - DNA, 35 bp, Synthetic construct
  source 1..35: mol_type = other DNA
ttcacctgcagtgccacctgacgtctaagaaacca

SEQ ID NO: 18 - DNA, 34 bp, Synthetic construct
  source 1..34: mol_type = other DNA
ttcacctgctcaggtggcacttttcggggaaatg

SEQ ID NO: 19 - DNA, 22 bp, Synthetic construct
  source 1..22: mol_type = other DNA
gcacttttcggggaaatgtgcg

SEQ ID NO: 20 - DNA, 31 bp, Synthetic construct
  source 1..31: mol_type = other DNA
ttggtctctccacctgacgtctaagaaacca

SEQ ID NO: 21 - DNA, 30 bp, Synthetic construct
  source 1..30: mol_type = other DNA
ttggtctctgtggcacttttcggggaaatg

SEQ ID NO: 22 - DNA, 31 bp, Synthetic construct
  source 1..31: mol_type = other DNA
ttcgtctctccacctgacgtctaagaaacca

SEQ ID NO: 23 - DNA, 30 bp, Synthetic construct
  source 1..30: mol_type = other DNA
ttcgtctctgtggcacttttcggggaaatg

SEQ ID NO: 24 - DNA, 27 bp, Synthetic construct
  source 1..27: mol_type = other DNA
ccgaaaagtgccacctgacgtctaaga

SEQ ID NO: 25 - DNA, 30 bp, Synthetic construct
  source 1..30: mol_type = other DNA
gataaccgtattaccgcctttgagtgagct

SEQ ID NO: 26 - DNA, 23 bp, Synthetic construct
  source 1..23: mol_type = other DNA
gggaagggtgggcgcgccgaccc

SEQ ID NO: 27 - DNA, 35 bp, Synthetic construct
  source 1..35: mol_type = other DNA
ggggaagggctccttcttaaagttaaacaaggcgg

SEQ ID NO: 28 - DNA, 30 bp, Synthetic construct
  source 1..30: mol_type = other DNA
agctcactcaaaggcggtaatacggttatc

SEQ ID NO: 29 - DNA, 29 bp, Synthetic construct
  source 1..29: mol_type = other DNA
gtttcttagacgtcaggtggcacttttcg

SEQ ID NO: 30 - DNA, 36 bp, Synthetic construct
  source 1..36: mol_type = other DNA
ttcgtctcttaatcgagcgcttccgaaaatgcaacg

SEQ ID NO: 31 - DNA, 34 bp, Synthetic construct
  source 1..34: mol_type = other DNA
ttcgtctcttcgacgttgtaaaacgacggccagt

SEQ ID NO: 32 - DNA, 36 bp, Synthetic construct
  source 1..36: mol_type = other DNA
ttggtctcttaatcgagcgcttccgaaaatgcaacg

SEQ ID NO: 33 - DNA, 34 bp, Synthetic construct
  source 1..34: mol_type = other DNA
ttggtctcttcgacgttgtaaaacgacggccagt

SEQ ID NO: 34 - DNA, 37 bp, Synthetic construct
  source 1..37: mol_type = other DNA
aagaagacaataatcgagcgcttccgaaaatgcaacg

SEQ ID NO: 35 - DNA, 35 bp, Synthetic construct
  source 1..35: mol_type = other DNA
aagaagacaatcgacgttgtaaaacgacggccagt

SEQ ID NO: 36 - DNA, 40 bp, Synthetic construct
  source 1..40: mol_type = other DNA
ttcacctgctttttaatcgagcgcttccgaaaatgcaacg

SEQ ID NO: 37 - DNA, 38 bp, Synthetic construct
  source 1..38: mol_type = other DNA
ttcacctgctttttcgacgttgtaaaacgacggccagt

SEQ ID NO: 38 - DNA, 27 bp, Synthetic construct
  source 1..27: mol_type = other DNA
taatcgagcgcttccgaaaatgcaacg

SEQ ID NO: 39 - DNA, 25 bp, Synthetic construct
  source 1..25: mol_type = other DNA
tcgacgttgtaaaacgacggccagt

SEQ ID NO: 40 - DNA, 26 bp, Synthetic construct
  source 1..26: mol_type = other DNA
aggcgagcgcttccgaaaatgcaacg

SEQ ID NO: 41 - DNA, 27 bp, Synthetic construct
  source 1..27: mol_type = other DNA
cctcgacgttgtaaaacgacggccagt

SEQ ID NO: 42 - DNA, 36 bp, Synthetic construct
  source 1..36: mol_type = other DNA
ttgctcttctaggcgagcgcttccgaaaatgcaacg

SEQ ID NO: 43 - DNA, 37 bp, Synthetic construct
  source 1..37: mol_type = other DNA
ttgctcttctcctcgacgttgtaaaacgacggccagt

SEQ ID NO: 44 - DNA, 33 bp, Synthetic construct
  source 1..33: mol_type = other DNA
gagacggatccctttttttagggatccgtctct

SEQ ID NO: 45 - DNA, 35 bp, Synthetic construct
  source 1..35: mol_type = other DNA
gaagagcgatccctttttttagggatcgctcttct

SEQ ID NO: 46 - DNA, 20 bp, Synthetic construct
  source 1..20: mol_type = other DNA
aaaagtgccacctgacgtct

SEQ ID NO: 47 - DNA, 28 bp, Synthetic construct
  source 1..28: mol_type = other DNA
gatcatttgtcagagcggctaaagcagg

SEQ ID NO: 48 - DNA, 27 bp, Synthetic construct
  source 1..27: mol_type = other DNA
tcaagatagccaaaaggggaggagtga

SEQ ID NO: 49 - DNA, 20 bp, Synthetic construct
  source 1..20: mol_type = other DNA
ccttttgctggccttttgct

SEQ ID NO: 50 - DNA, 16 bp, Synthetic construct
  source 1..16: mol_type = other DNA
ctgcaaccggtgaatg

SEQ ID NO: 51 - DNA, 16 bp, Synthetic construct
  source 1..16: mol_type = other DNA
aagagaccggtctaac

SEQ ID NO: 52 - DNA, 58 bp, Synthetic construct
  source 1..58: mol_type = other DNA
ctaacttagttttccaactgttttccacgcctactgcataaccggatacacggatggt

SEQ ID NO: 53 - DNA, 26 bp, Synthetic construct
  source 1..26: mol_type = other DNA
actgcataaccggatacacggatggt

SEQ ID NO: 54 - DNA, 28 bp, Synthetic construct
  source 1..28: mol_type = other DNA
atctcgctctcctgcgaatcttcaatcc

SEQ ID NO: 55 - DNA, 56 bp, Synthetic construct
  source 1..56: mol_type = other DNA
aactctgccatccggcatttccccgcaaatctcgctctcctgcgaatcttcaatcc

SEQ ID NO: 56 - DNA, 50 bp, Synthetic construct
  source 1..50: mol_type = other DNA
gcggattgaagattcgcaggagagcgagatttgcggggaaatgccggatg

SEQ ID NO: 57 - DNA, 22 bp, Synthetic construct
  source 1..22: mol_type = other DNA
atttgcggggaaatgccggatg

SEQ ID NO: 58 - DNA, 27 bp, Synthetic construct
  source 1..27: mol_type = other DNA
ggtggatgaagcggaattgacaaaagc

SEQ ID NO: 59 - DNA, 57 bp, Synthetic construct
  source 1..57: mol_type = other DNA
ctccacaggatgatttcgtcagctgacgtcggtggatgaagcggaattgacaaaagc

SEQ ID NO: 60 - DNA, 22 bp, Synthetic construct
  source 1..22: mol_type = other DNA
tgctgttccggagtcagagagt

SEQ ID NO: 61 - DNA, 22 bp, Synthetic construct
  source 1..22: mol_type = other DNA
cagcaaaccgcaaagcgtacca

SEQ ID NO: 62 - DNA, 24 bp, Synthetic construct
  source 1..24: mol_type = other DNA
ttttggactgccgctcgctggcga

SEQ ID NO: 63 - DNA, 24 bp, Synthetic construct
  source 1..24: mol_type = other DNA
aaactcgccagcgagcggcagtcc

SEQ ID NO: 64 - DNA, 23 bp, Synthetic construct
  source 1..23: mol_type = other DNA
acagacgaagaatccatgacgtc

SEQ ID NO: 65 - DNA, 24 bp, Synthetic construct
  source 1..24: mol_type = other DNA
gcgctgccatcttgctaaacctac

SEQ ID NO: 66 - DNA, 23 bp, Synthetic construct
  source 1..23: mol_type = other DNA
ggactgccgctcgctggcgacgg

SEQ ID NO: 67 - DNA, 23 bp, Synthetic construct
  source 1..23: mol_type = other DNA
tcgccgctttcggcgcatcgtgg

SEQ ID NO: 68 - DNA, 23 bp, Synthetic construct
  source 1..23: mol_type = other DNA
tggctgcaaccattttgtgctgg

SEQ ID NO: 69 - DNA, 23 bp, Synthetic construct
  source 1..23: mol_type = other DNA
attttgtgctggcaaatcagtgg

SEQ ID NO: 70 - DNA, 23 bp, Synthetic construct
  source 1..23: mol_type = other DNA
gctcgctggcgacggtctgaagg

SEQ ID NO: 71 - DNA, 23 bp, Synthetic construct
  source 1..23: mol_type = other DNA
gaaggtttcccagccgaggctgg

SEQ ID NO: 72 - DNA, 23 bp, Synthetic construct
  source 1..23: mol_type = other DNA
agagttgggtgacgttaaagtgg

SEQ ID NO: 73 - DNA, 23 bp, Synthetic construct
  source 1..23: mol_type = other DNA
aaggcgtggtgtttttgttgtgg

SEQ ID NO: 74 - DNA, 23 bp, Synthetic construct
  source 1..23: mol_type = other DNA
actgtgttgacggtacgcgcagg

SEQ ID NO: 75 - DNA, 23 bp, Synthetic construct
  source 1..23: mol_type = other DNA
ggcgtggtgtttttgttgtgggg

SEQ ID NO: 76 - DNA, 23 bp, Synthetic construct
  source 1..23: mol_type = other DNA
ctcggccaggatccttatcacgg

SEQ ID NO: 77 - DNA, 23 bp, Synthetic construct
  source 1..23: mol_type = other DNA
cacggaccgggacaggcgcatgg

SEQ ID NO: 78 - DNA, 23 bp, Synthetic construct
  source 1..23: mol_type = other DNA
aatccacgatgcgccgaaagcgg

SEQ ID NO: 79 - DNA, 23 bp, Synthetic construct
  source 1..23: mol_type = other DNA
cttgaaagctgggcgcgtcaggg

SEQ ID NO: 80 - DNA, 23 bp, Synthetic construct
  source 1..23: mol_type = other DNA
gacgttaaagtggtgattctcgg

SEQ ID NO: 81 - DNA, 23 bp, Synthetic construct
  source 1..23: mol_type = other DNA
gcgttaaagacatctttttgtgg

SEQ ID NO: 82 - DNA, 23 bp, Synthetic construct
  source 1..23: mol_type = other DNA
aatcaggctgatcactttatcgg

SEQ ID NO: 83 - DNA, 23 bp, Synthetic construct
  source 1..23: mol_type = other DNA
tgactggatgccagtattaccgg

SEQ ID NO: 84 - DNA, 23 bp, Synthetic construct
  source 1..23: mol_type = other DNA
tgacggaggaatggcaatgccgg

SEQ ID NO: 85 - DNA, 23 bp, Synthetic construct
  source 1..23: mol_type = other DNA
cggtgaaggtttcccagccgagg

SEQ ID NO: 86 - DNA, 23 bp, Synthetic construct
  source 1..23: mol_type = other DNA
cgtcacccaactctgtaaagcgg

SEQ ID NO: 87 - DNA, 23 bp, Synthetic construct
  source 1..23: mol_type = other DNA
tgattagggcgggtgaagcccgg

SEQ ID NO: 88 - DNA, 23 bp, Synthetic construct
  source 1..23: mol_type = other DNA
gacgccggactgccgctcgctgg

SEQ ID NO: 89 - DNA, 23 bp, Synthetic construct
  source 1..23: mol_type = other DNA
ttcccagccgaggctggcgtggg

SEQ ID NO: 90 - DNA, 23 bp, Synthetic construct
  source 1..23: mol_type = other DNA
ccggtccgtgataaggatcctgg

SEQ ID NO: 91 - DNA, 23 bp, Synthetic construct
  source 1..23: mol_type = other DNA
aggcgtggtgtttttgttgtggg

SEQ ID NO: 92 - DNA, 23 bp, Synthetic construct
  source 1..23: mol_type = other DNA
tcactctctgccggtaatactgg

SEQ ID NO: 93 - DNA, 23 bp, Synthetic construct
  source 1..23: mol_type = other DNA
attcccacgccagcctcggctgg

SEQ ID NO: 94 - DNA, 23 bp, Synthetic construct
  source 1..23: mol_type = other DNA
tttcccagccgaggctggcgtgg

SEQ ID NO: 95 - DNA, 23 bp, Synthetic construct
  source 1..23: mol_type = other DNA
aatcagtggctggaacaacgtgg

SEQ ID NO: 96 - DNA, 23 bp, Synthetic construct
  source 1..23: mol_type = other DNA
gagctggaaaatactattccggg

SEQ ID NO: 97 - DNA, 23 bp, Synthetic construct
  source 1..23: mol_type = other DNA
agagctggaaaatactattccgg

SEQ ID NO: 98 - DNA, 23 bp, Synthetic construct
  source 1..23: mol_type = other DNA
ttaaagacatctttttgtggtgg

SEQ ID NO: 99 - DNA, 23 bp, Synthetic construct
  source 1..23: mol_type = other DNA
ggcttcacccgccctaatcatgg

SEQ ID NO: 100 - DNA, 10 bp, Synthetic construct
  source 1..10: mol_type = other DNA
tttcacctgc

SEQ ID NO: 101 - DNA, 10 bp, Synthetic construct
  source 1..10: mol_type = other DNA
aattggcaca

SEQ ID NO: 102 - DNA, 10 bp, Synthetic construct
  source 1..10: mol_type = other DNA
accaggaatt

SEQ ID NO: 103 - DNA, 10 bp, Synthetic construct
  source 1..10: mol_type = unassigned DNA
atagccggtt

SEQ ID NO: 104 - DNA, 10 bp, Synthetic construct
  source 1..10: mol_type = other DNA
tgcttccaag

SEQ ID NO: 105 - DNA, 10 bp, Synthetic construct
  source 1..10: mol_type = other DNA
ccgaaggttc

SEQ ID NO: 106 - DNA, 41 bp, Synthetic construct
  source 1..41: mol_type = other DNA
ggtgcaggtggatccctttttttagggatccacctgcacct

SEQ ID NO: 107 - DNA, 51 bp, Synthetic construct
  source 1..51: mol_type = other DNA
ggtcttcacatcgcggatccctttttttagggatccgcgatgtgaagacct

SEQ ID NO: 108 - DNA, 33 bp, Synthetic construct
  source 1..33: mol_type = other DNA
gagaccgatccctttttttagggatcggtctct

SEQ ID NO: 109 - DNA, 33 bp, Synthetic construct; identical to SEQ ID NO: 44
  source 1..33: mol_type = other DNA

SEQ ID NO: 110 - DNA, 35 bp, Synthetic construct; identical to SEQ ID NO: 45
  source 1..35: mol_type = other DNA

SEQ ID NO: 111 - DNA, 22 bp, Synthetic construct
  source 1..22: mol_type = other DNA
acgatcatggcgttcttcatcc

SEQ ID NO: 112 - DNA, 31 bp, Synthetic construct
  source 1..31: mol_type = other DNA
actctaattcttgagccttatccatatgact

SEQ ID NO: 113 - DNA, 26 bp, Synthetic construct
  source 1..26: mol_type = other DNA
gagttggccgttgctgaactgaatgc

SEQ ID NO: 114 - DNA, 24 bp, Synthetic construct
  source 1..24: mol_type = other DNA
gatgcgagggtgatccgatcgaac

SEQ ID NO: 115 - DNA, 24 bp, Synthetic construct
  source 1..24: mol_type = other DNA
catctcaggtgaccatagacacca

SEQ ID NO: 116 - DNA, 26 bp, Synthetic construct
  source 1..26: mol_type = other DNA
aataactacccaacgcaacgcttctg

SEQ ID NO: 117 - DNA, 29 bp, Synthetic construct
  source 1..29: mol_type = other DNA
tattttaccgcatcggctctactcacact

SEQ ID NO: 118 - DNA, 26 bp, Synthetic construct
  source 1..26: mol_type = other DNA
aaagtgcgttaccgtttcaggttgca

SEQ ID NO: 119 - DNA, 29 bp, Synthetic construct
  source 1..29: mol_type = other DNA
cttttaccagtctttcggtcagttcaccg

SEQ ID NO: 120 - DNA, 34 bp, Synthetic construct
  source 1..34: mol_type = other DNA
agaattgatttcagcgagacggtatttttctcgg

SEQ ID NO: 121 - DNA, 22 bp, Synthetic construct
  source 1..22: mol_type = other DNA
ttctgaccgatccgccgtcact

SEQ ID NO: 122 - DNA, 26 bp, Synthetic construct
  source 1..26: mol_type = other DNA
ctcatcataactgagtggggccaggg

SEQ ID NO: 123 - DNA, 33 bp, Synthetic construct
  source 1..33: mol_type = other DNA
tgagtacaacgtgctgattgaaaaaggttgtct

SEQ ID NO: 124 - DNA, 28 bp, Synthetic construct
  source 1..28: mol_type = other DNA
gcaacggtttacaaagtcagcgtcaaca

SEQ ID NO: 125 - DNA, 34 bp, Synthetic construct
  source 1..34: mol_type = other DNA
cgatcatggcgttcttcatcctctatttctgtct

SEQ ID NO: 126 - DNA, 30 bp, Synthetic construct
  source 1..30: mol_type = other DNA
tattttctgcattcagttcagcaacggcca

SEQ ID NO: 127 - DNA, 37 bp, Synthetic construct
  source 1..37: mol_type = other DNA
atactgaagctgaaaatgaaattctgctggatgtcac

SEQ ID NO: 128 - DNA, 23 bp, Synthetic construct
  source 1..23: mol_type = other DNA
gagggtgatccgatcgaacagcc

SEQ ID NO: 129 - DNA, 27 bp, Synthetic construct
  source 1..27: mol_type = other DNA
ctcgcatctcaggtgaccatagacacc

SEQ ID NO: 130 - DNA, 23 bp, Synthetic construct
  source 1..23: mol_type = other DNA
aacgcttctgaaatgccccagct

SEQ ID NO: 131 - DNA, 30 bp, Synthetic construct
  source 1..30: mol_type = other DNA
gttgcgttgggtagttattttaccgcatcg

SEQ ID NO: 132 - DNA, 24 bp, Synthetic construct
  source 1..24: mol_type = other DNA
tgcgttaccgtttcaggttgcagg

SEQ ID NO: 133 - DNA, 31 bp, Synthetic construct
  source 1..31: mol_type = other DNA
gcacttttaccagtctttcggtcagttcacc

SEQ ID NO: 134 - DNA, 23 bp, Synthetic construct
  source 1..23: mol_type = other DNA
ggatcaatcgtccagacctgcgc

SEQ ID NO: 135 - DNA, 26 bp, Synthetic construct
  source 1..26: mol_type = other DNA
tcccgagaaaaataccgtctcgctga

SEQ ID NO: 136 - DNA, 27 bp, Synthetic construct
  source 1..27: mol_type = other DNA
agggctttgctgctttttgtgaatcgg

SEQ ID NO: 137 - DNA, 26 bp, Synthetic construct
  source 1..26: mol_type = other DNA
cctggccccactcagttatgatgagt

SEQ ID NO: 138 - DNA, 30 bp, Synthetic construct
  source 1..30: mol_type = other DNA
caacggtttacaaagtcagcgtcaacatcg

SEQ ID NO: 139 - DNA, 28 bp, Synthetic construct
  source 1..28: mol_type = other DNA
gactcactataggggaattgtgagcgga

SEQ ID NO: 140 - DNA, 20 bp, Synthetic construct
  source 1..20: mol_type = other DNA
tcggccccatactgacggga

SEQ ID NO: 141 - DNA, 20 bp, Synthetic construct
  source 1..20: mol_type = other DNA
ttgcgggggcagtctggaag

SEQ ID NO: 142 - DNA, 20 bp, Synthetic construct
  source 1..20: mol_type = other DNA
cgacgacgcctgtggggaaa

SEQ ID NO: 143 - DNA, 21 bp, Synthetic construct
  source 1..21: mol_type = other DNA
tacaatcgcagtggccgggag

SEQ ID NO: 144 - DNA, 20 bp, Synthetic construct
  source 1..20: mol_type = other DNA
cagctccgcgaagtcgctct

SEQ ID NO: 145 - DNA, 25 bp, Synthetic construct
  source 1..25: mol_type = other DNA
agagctgccaggaaacagctatgac

SEQ ID NO: 146 - DNA, 24 bp, Synthetic construct
  source 1..24: mol_type = other DNA
acgacgttgtaaaacgacggccag